(19) United States
(12) Patent Application Publication
(10) Pub. No.: US 2025/0188448 A1
(43) Pub. Date: Jun. 12, 2025

(54) METHODS OF IDENTIFYING PROXIMITY EFFECTOR POLYPETIDES AND METHODS OF USE THEREOF

(71) Applicant: THE GOVERNING COUNCIL OF THE UNIVERSITY OF TORONTO, Toronto (CA)

(72) Inventors: Mikko TAIPALE, Toronto (CA); Akashdeep DHILLON, Brampton (CA); Nader ALERASOOL, Toronto (CA); Lamisa MIZAN, Richmond Hill (CA); Juline POIRSON, Toronto (CA)

(21) Appl. No.: 18/856,491

(22) PCT Filed: Apr. 14, 2023
(86) PCT No.: PCT/CA2023/050511

§ 371 Date: Oct. 11, 2024

Related U.S. Application Data
(60) Provisional application No. 63/331,078, filed on Apr. 14, 2022.

(51) Int. Cl. C12N 15/10; C07K 14/47

(52) U.S. Cl. CPC C12N 15/1086; C07K 14/47; C07K 2319/00

(57) ABSTRACT

Disclosed herein is a method of identifying a proximity effector polypeptide, the method comprising: transducing an ORFeome library into a plurality of cells, the ORFeome library encoding a plurality of ORFs, wherein each of the ORFs is fused to a targeting moiety that binds or can be induced to bind to the target polypeptide directly or indirectly; expressing the plurality of ORFs of the ORFeome library in the transduced plurality of cells, under conditions for the targeting moiety to interact with the target polypeptide; and determining whether any of the plurality of ORFs is a proximity effector polypeptide by measuring abundance, wherein an ORF encodes a proximity effector polypeptide when the ORF increases or decreases the target polypeptide abundance compared to control or is depleted or enhanced in the transduced plurality of cells compared to the control.

### CROSS-REFERENCE TO RELATED APPLICATIONS

This application claims the benefit of priority to U.S. Provisional Application No. 63/331,078, filed Apr. 14, 2022, the contents of which is incorporated herein by reference in its entirety.

### INCORPORATION OF SEQUENCE LISTING

A computer readable form of the Sequence Listing “2223-P67134PC00_Sequence_Listing” (37,186 bytes) created on Apr. 13, 2023, is herein incorporated by reference.

### FIELD

The present disclosure relates to proximity effectors, for example for targeted protein degradation and stabilization, in particular to methods of identifying proximity effector polypeptides and using the proximity factors in screens and methods for example for targeted protein degradation or stabilization.

### BACKGROUND

Targeted protein degradation (TPD) has emerged as one of the most promising innovations in drug development (Burslem and Crews, 2020). The idea of TPD is to selectively induce target protein degradation instead of just inhibiting or activating the target. TPD can be brought about by either heterobifunctional molecules known as PROteolysis-TArgeting Chimeras (PROTACs) or “molecular glues”. PROTACs consist of two covalently linked protein-binding moieties: one binds a target protein while the other binds an E3 ubiquitin ligase, bringing the target protein to close proximity with the ligase. This leads to ubiquitination of the target protein followed by proteasome-dependent degradation. Molecular glues work in a similar way, but they directly contact both the E3 ligase and the target protein, inducing a non-native protein/protein interaction that results in target degradation.

The concept of PROTACs and molecular glues exploits the promiscuity of many (but not all) E3 ligases: their substrate specificity is largely determined by physical proximity to the substrate rather than its intrinsic features. TPD has several advantages over traditional small-molecule inhibitors, including the ability of one molecule to degrade several protein molecules, thus increasing the potency of the drug. Moreover, they expand the target space of small molecules, as they can target any druggable domain of a protein rather than just the enzymatically active domain.

Despite the high therapeutic potential of PROTACs and molecular glues, the major barrier in their development is that only a handful of known E3 ligases have been used with the approach. Indeed, most current glues and PROTACs harness only two E3s (i.e., E3 ligases): the thalidomide target Cereblon (CRBN) and the tumor suppressor VHL. This is a significant challenge because compounds exploiting these two E3s only work with some target proteins but not others, in a highly unpredictable manner. Identifying novel proteins that could work more robustly and predictably would clear this roadblock in the development of new degraders and potentiate drug discovery in many therapeutic areas.

Although many groups are actively trying to characterize E3 ligases that are suitable for PROTAC and molecular glue development, most approaches rely on a limited number of previously well-characterized E3s rather than the full spectrum of over 600 ubiquitin ligases in the human genome (Burslem and Crews, 2020). Furthermore, this is likely an underestimate, as E3 annotation is based on their characteristic protein domains (e.g., RING finger, F-box) rather their molecular function. For example, kinases, G proteins, and metabolic enzymes can also function as substrate adaptors for E3s and other ubiquitin-modifying complexes. It is likely that there are many other unexpected proteins that functionally act as E3s, expanding the toolkit for targeted protein degradation.

It is also possible that other protein quality control pathways could be exploited. Protein turnover is regulated by multiple other mechanisms, including selective macroautophagy, mitophagy, chaperone-mediated autophagy, general proteases, and even extracellular secretion. Their specificity is partly regulated by receptors that determine the fate of the substrate protein, making them potential pathways for targeted protein degradation. However, these avenues have remained completely unexplored.

More broadly, induced-proximity based therapeutics are not limited to only protein degradation. Indeed, much of biology is driven by protein/protein interactions that assemble in a dynamic manner, and many natural compounds such as plant hormones function by inducing specific protein/protein interactions (Gerry and Schreiber, 2020). Thus, compounds that rewire protein/protein interactions have an enormous potential in next-generation therapeutics. Indeed, several proof-of-concept studies have showcased the potential of inducing protein stabilization with deubiquitinases (DUBTACs), protein dephosphorylation with phosphatases (PhoRCs), and autophagy-inducing compounds with AUTACs (Henning et al., 2021; Takahashi et al., 2019; Yamazoe et al., 2020).

However, one of the major roadblocks in developing induced proximity drugs is the “protein pair problem”. If one wants to regulate the function of a given target, how to identify the ideal effector protein, given that there are over 600 E3 ligases, 500 kinases, 100 deubiquitinases, and thousands of other proteins that might provide a beneficial functional outcome.

### SUMMARY

Polypeptide proximity effectors such as those that promote degradation or stabilization of a target protein in a proximity dependent manner are identified herein.

An aspect of the disclosure includes a method of identifying a proximity effector polypeptide, the method comprising:
transducing an ORFeome library into a plurality of cells, the ORFeome library encoding a plurality of ORFs, wherein each of the ORFs is fused to a targeting moiety that binds or can be induced to bind to the target polypeptide directly or indirectly;
expressing the plurality of ORFs of the ORFeome library in the transduced plurality of cells, under conditions for the targeting moiety to interact with the target polypeptide; and
determining whether any of the plurality of ORFs is a proximity effector polypeptide by measuring abundance, optionally total abundance, cell surface abundance or subcellular abundance, of the target polypeptide in cells expressing any of the plurality of ORFs compared to a control and/or detecting whether any of the plurality of ORFs is depleted or enhanced in the transduced plurality of cells compared to a control;
wherein the transduced plurality of cells recombinantly expresses the target polypeptide and optionally expresses a first fluorescent polypeptide, wherein the target polypeptide is a second fluorescent polypeptide, endogenous protein, or a fusion polypeptide fused to a second fluorescent polypeptide, an epitope tag, an antibiotic resistance protein and/or a negative selection marker; and wherein an ORF encodes a proximity effector polypeptide when the ORF increases or decreases the target polypeptide abundance compared to control, or is depleted or enhanced in the transduced plurality of cells compared to the control.

In some embodiments, measuring abundance of the target polypeptide comprises i) determining whether the target polypeptide expressed in the plurality of cells has decreased or has increased relative to a control (e.g. total abundance or in a subcellular fraction), or ii) determining whether any of the plurality of ORFs increased cell surface levels of the target polypeptide compared to a control.

In some embodiments, the proximity effector polypeptide is a degrader of the target polypeptide when the target polypeptide is decreased compared to a control or the proximity effector polypeptide is a stabilizer of the target polypeptide when the target polypeptide has increased compared to a control. In some embodiments, the proximity effector polypeptide is a lethal polypeptide when depleted or decreased in the transduced plurality of cells or is a growth inducing polypeptide when enhanced in the transduced plurality of cells compared to a control. In some embodiments, the proximity effector polypeptide is a protein trafficking polypeptide when the proximity effector increases cell surface levels of the target polypeptide compared to a control.

In some embodiments, the target polypeptide is or is fused to a second fluorescent polypeptide, the determining comprises isolating a fraction of the plurality of cells with a selected second fluorescent polypeptide: first fluorescent polypeptide ratio and sequencing one or more of the plurality of ORFs in the fraction; wherein when the target polypeptide is fused to an epitope tag, the determining comprises measuring abundance of the target polypeptide with an epitope tag binding protein, optionally an antibody; wherein when the target polypeptide is an endogenous target, the determining comprises measuring abundance, optionally total abundance, cell surface abundance or subcellular abundance, of the target polypeptide with a target polypeptide binding protein, optionally an antibody; wherein when the target polypeptide is fused to the antibiotic selection protein, the determining comprises isolating a fraction of the plurality of cells that survive antibiotic treatment and sequencing one or more of the ORFs in the fraction; or wherein when the target polypeptide is fused to the negative selection marker, optionally thymidine kinase, the determining comprises isolating a fraction of the plurality of cells that survive negative selection treatment and sequencing one or more of the ORFs in the fraction.

In some embodiments, the plurality of cells is a cell line and the method further comprises generating the cell line by introducing a nucleic acid encoding the target polypeptide, and optionally the first fluorescent polypeptide, optionally wherein the target polypeptide and first fluorescent polypeptide are in a construct comprising an IRES or cleavage site therebetween. In some embodiments, the ratio of the second fluorescent polypeptide to the first fluorescent polypeptide is determined using a method comprising flow cytometry. In some embodiments, the first or second fluorescent polypeptide(s) is/are RFP, YFP, mCherry, mCitrine, mNeonGreen, mScarlet, BFP and/or GFP. In some embodiments, the first fluorescent polypeptide is GFP and the second fluorescent polypeptide is BFP or the first fluorescent polypeptide is BFP and the second fluorescent polypeptide is GFP. In some embodiments, the target polypeptide is fused to the antibiotic resistance protein or the negative selection marker. In some embodiments, the negative selection marker is thymidine kinase, mutant deoxycytodine kinase or thymidylate kinase. In some embodiments, when the negative selection marker is thymidine kinase the method comprises treating the plurality of cells with ganciclovir during the step of expressing the plurality of ORFs of the ORFeome library, under conditions for the targeting moiety to interact with the target polypeptide, wherein survival of a cell when exposed to ganciclovir indicates that the level of the target polypeptide is decreased.

In some embodiments, the ORF identified in a cell that survives antibiotic treatment is a proximity effector polypeptide. In some embodiments, the antibiotic resistance protein is puromycin acetyltransferase, neomycin phosphotransferase, blasticidin deaminase, or hygromycin kinase. In some embodiments, the method comprises treating the plurality of cells with puromycin during the step of expressing the plurality of ORFs of the ORFeome library, under conditions for the targeting moiety to interact with the target polypeptide.

In some embodiments, the determining comprises measuring growth of the transduced plurality of cells and ORF(s) identified in a cell of the transduced plurality of cells that enhance(s) or decrease(s) cell proliferation compared to a control is/are a proximity effector polypeptide. In some embodiments, the plurality of cells are transduced to maintain >300, >400 or >500 fold coverage of the ORFeome library.

In some embodiments, the method further comprises testing identified proximity effector polypeptides in an individual proximity effector assay, optionally when the proximity effector polypeptide is identified as a stabilizer or degrader, expressing the putative proximity effector polypeptide identified as the stabilizer or degrader in a test cell expressing the target polypeptide and determining whether the level of the target polypeptide in the test cell has decreased or has increased.

In some embodiments, the proximity effector polypeptide is a plurality of proximity effector polypeptides.

In some embodiments, the method described herein is for identifying a proximity effector polypeptide that is a lethal polypeptide or a growth inducing polypeptide, and the determining comprises determining whether the ORF has caused death or induced proliferation of at least one cell of the transduced plurality of cells and/or is depleted or enhanced in the transduced plurality of cells compared to a control, wherein the proximity effector polypeptide is a lethal polypeptide when the proximity effector polypeptide causes death in at least one cell of the transduced plurality of cells and/or wherein it is depleted in the transduced plurality of cells and the putative proximity effector polypeptide is a growth inducing polypeptide when the proximity effector polypeptide induces proliferation in at least one cell and/or is enhanced in the transduced plurality of cells compared to a control.

In some embodiments, the determining comprises identifying the ORFs depleted in the plurality of cells, optionally comprising sequencing the plurality of ORFs in the transduced plurality of cells that survived and comparing a reference of the plurality of ORFs in the ORFeome library to determine ORFs that are or are not present.

In some embodiments, the target polypeptide is an oncogenic polypeptide, a regulator of apoptosis, a regulator of autophagy, or a regulator of mitophagy. In some embodiments, the target polypeptide is a RAS polypeptide, optionally KRAS, MYC, or EWSR-FLI1.

In some embodiments, the method described herein is for identifying a proximity effector polypeptide that is a protein trafficking polypeptide, wherein the determining comprises measuring cell surface levels of the target polypeptide, wherein the proximity effector polypeptide is a protein trafficking polypeptide when it increases the cell surface levels of the target polypeptide. In some embodiments, the target polypeptide is a MHC class I polypeptide. In some embodiments, the target polypeptide is a mutant cell surface polypeptide, optionally provided Table 2, preferably CFTR delta508.

In some embodiments, the target polypeptide comprises EGFP-AB1, Rluc, FUS S525L, NRAS, DNAJA3, BRAF, LAMP1, TDP43 Q311K, CD63, H2B, EGFR, DNAJB11, WDR5, RAS, MYC, or EWSR-FLI1, EWSR1, SMARCA2/4, or PARP1, PD1/PD-L1, JAK, FUS, TDP43, a-synuclein, amyloid beta precursor protein, HTT, prion protein, p53, PTEN, a CFTR variant, and/or dystrophin variant.

In some embodiments, the targeting moiety is a nanobody, ligand, interaction peptide or an antibody that binds the target polypeptide. In some embodiments, the targeting moiety is the nanobody. In some embodiments, the targeting moiety is an interaction peptide (or complementary interaction peptide) selected from ABI1, FKBP, FRB, mutant FRB, GID1, GAI, and/or PYR1. In some embodiments, the target polypeptide is a fusion polypeptide comprising the interaction peptide that interacts with the targeting moiety. In some embodiments, the fusion polypeptide comprises ABI1, FKBP, FRB, mutant, FRB, GID1, GAI, PYL1, Alfa tag and/or PYR1.

In some embodiments, method comprises use of a chemical inducer. In some embodiments, when the target polypeptide comprises ABI1, the targeting moiety comprises PYR1 or PYL1, and the chemical inducer is mandipropamid or abscisic acid. In some embodiments, when the target polypeptide comprises PYR1, the targeting moiety comprises ABI1, and the chemical inducer is mandipropamid or abscisic acid. In some embodiments, when the target polypeptide comprises ABI1, the targeting moiety comprises PYL1, and the chemical inducer is abscisic acid. In some embodiments, when the target polypeptide comprises FKBP, the targeting moiety comprises FRB, and the chemical inducer is rapamycin. In some embodiments, when the target polypeptide comprises FRB, the targeting moiety comprises FKBP, and the chemical inducer is rapamycin. In some embodiments, when the target polypeptide comprises FKBP, the targeting moiety comprises mutant FRB, and the chemical inducer is a rapalog, optionally AP21967. In some embodiments, when the target polypeptide comprises mutant FRB, the targeting moiety comprises FKBP, and the chemical inducer is a rapalog, optionally AP21967. In some embodiments, when the target polypeptide comprises GID1, the targeting moiety comprises GAI, and the chemical inducer is gibberellic acid. In some embodiments, when the target polypeptide comprises mutant GAI, the targeting moiety comprises GID1, and the chemical inducer is gibberellic acid.

In some embodiments, the method further comprises performing a screening assay for identifying a ligand, optionally a small molecule binder, for at least one recombinant proximity effector polypeptide identified.

Another aspect of the disclosure includes a screening assay for identifying a ligand, optionally a small molecule binder, of at least one recombinant proximity effector polypeptide, the screening assay comprising:
contacting the at least one recombinant proximity effector polypeptide with a small molecule library optionally in a high-throughput screening assay, wherein the proximity effector polypeptide is selected from Table 4, 5, 6 or 7,
assessing whether binding has occurred between the recombinant proximity effector polypeptide and one or more small molecule(s) of the small molecule library,
wherein the one or more molecule(s) which have bound to the at least one recombinant proximity effector polypeptide is a ligand, optionally a small molecule binder, of the at least one recombinant proximity effector polypeptide; preferably wherein the proximity effector polypeptide is selected from GMCL1, FBXL15, PJA1, RNF115, DZIP3, RNF125, FBXO3, RNF185, RNF8, RNF183, RCHY1, KBTBD7, TRIM31, CISH, SOCS5, TRIM39, RNF144B, FBXO40, KLHL6, FBXO11, GAN, FBXL14, FBXW5, RNF111, FBXL12, BTRC, or RNF126 or selected from FBXL12, FBXL14, FBXL15, KLHDC2, KLHL6, KBTBD7, ZER1, UBE2B or KLHL40.

In some embodiments, the assay further comprises contacting a target polypeptide with the small molecule library and determining whether binding has occurred between the target polypeptide and one or more small molecule(s) of the small molecule library. In some embodiments, the assessing step is performed using surface plasmon resonance (SPR), nuclear magnetic resonance (NMR) spectroscopy, differential scanning fluorimetry (DSF), thermal shift assay (TSA), isothermal titration calorimetry (ITC), microscale thermophoresis (MST), biolayer interferometry (BLI), X-ray crystallography, DNA-Encoded Library (DEL) screens, affinity selection-mass spectrometry (AS-MS), or covalent fragment screens.

In some embodiments, the assay further comprises identifying whether the small molecule binder of the recombinant proximity effector polypeptide is a molecular glue by determining whether the recombinant proximity effector and the target polypeptide interact in the presence of the small molecule binder. In some embodiments, the determining step is performed using a luciferase complementation, a yeast two-hybrid assay, an AlphaScreen, a yeast mating based interaction assay, fluorescence resonance energy transfer microscopy (FRET), or time-resolved FRET (TR-FRET), wherein the small molecule binder is a molecular glue if it interacts or is capable of interacting with the recombinant proximity effector polypeptide and the target polypeptide simultaneously.

In some embodiments, the at least one recombinant proximity effector polypeptide has been identified using the methods described herein.

Another aspect of the disclosure includes a method of making a heterobifunctional molecule, the method comprising:
identifying a ligand, optionally a small molecule binder, of an effector polypeptide and a ligand, optionally a small molecule binder, of a target polypeptide using the methods described herein, and
coupling the small molecule binder of the effector polypeptide and the small molecule binder of the target polypeptide optionally via a linker.

In some embodiments, the method further comprises assessing whether the effector polypeptide and the target polypeptide interact in the presence of the heterobifunctional molecule. In some embodiments, the assessing step is performed using a luciferase complementation, a yeast two-hybrid assay, an AlphaScreen, a yeast mating based interaction assay, fluorescence resonance energy transfer microscopy (FRET), or time-resolved FRET (TR-FRET).

Another aspect of the disclosure includes a process for modulating a target polypeptide in at least one cell, the method comprising: expressing the proximity effector polypeptide provided in Table 4, 5, 6 or 7 in the at least one cell, the at least one proximity effector polypeptide being fused to a targeting moiety, preferably wherein the proximity effector polypeptide is selected from GMCL1, FBXL15, PJA1, RNF115, DZIP3, RNF125, FBXO3, RNF185, RNF8, RNF183, RCHY1, KBTBD7, TRIM31, CISH, SOCS5, TRIM39, RNF144B, FBXO40, KLHL6, FBXO11, GAN, FBXL14, FBXW5, RNF111, FBXL12, BTRC, or RNF126 or selected from FBXL12, FBXL14, FBXL15, KLHDC2, KLHL6, KBTBD7, ZER1, UBE2B or KLHL40.

In some embodiments, the proximity effector polypeptide is at least one degrader. In some embodiments, the proximity effector polypeptide is at least one stabilizer. In some embodiments, the at least one degrader polypeptide is selected from UBE2B, UBE2A, FBXL12, FBXL14, FBXL15, GABARAP, GABARAPL2, MAP1LC3A, KLHL6, KBTBD7, ZER1 and/or KLHDC2. In some embodiments, the at least one degrader is selected from GMCL1, FBXL15, PJA1, RNF115, DZIP3, RNF125, FBXO3, RNF185, RNF8, RNF183, RCHY1, KBTBD7, TRIM31, CISH, SOCS5, TRIM39, RNF144B, FBXO40, KLHL6, FBXO11, GAN, FBXL14, FBXW5, RNF111, FBXL12, BTRC, ZER1 and/or RNF126. In some embodiments, the at least one degrader polypeptide is selected from FBXL12, FBXL14, FBXL15, KLHDC2, KLHL6, KBTBD7, ZER1 and/or UBE2B. In some embodiments, the at least one stabilizer polypeptide is selected from KLHL40, KLHL41, DDI1, and/or PRPS2, preferably KLHL40.

In some embodiments, the target polypeptide is an oncogene polypeptide, oncogenic fusion polypeptide, synthetic lethal target, immunology/immune-oncology target, dominant gain-of-function disease variant, tumor suppressor, or unstable disease variant. In some embodiments, the oncogene polypeptide or oncogenic fusion polypeptide is or comprises RAS, MYC, or EWSR-FLI1. In some embodiments, the synthetic lethal target is EWSR1, SMARCA2/4, or PARP1. In some embodiments, the immunology/immune-oncology target is PD1/PD-L1 or JAK. In some embodiments, the dominant gain-of-function disease variant is FUS, TDP43, a-synuclein, amyloid beta precursor protein, HTT, or prion protein. In some embodiments, the tumor suppressor is p53 or PTEN. In some embodiments, the unstable disease variant is CFTR mutations or dystrophin variants.

In some embodiments, the proximity effector is KLHL40 or KLHL41 and the target polypeptide is a loss of stability variant in muscular dystrophy. In some embodiments, the target polypeptide is BCR-ABL. In some embodiments, the targeting moiety is a nanobody, ligand, interaction peptide or an antibody.

Another aspect of the disclosure includes a fusion polypeptide comprising a proximity effector polypeptide selected from Table 4, 5, 6 or 7 and a targeting moiety that binds a target polypeptide, preferably wherein the proximity effector polypeptide is selected from GMCL1, FBXL15, PJA1, RNF115, DZIP3, RNF125, FBXO3, RNF185, RNF8, RNF183, RCHY1, KBTBD7, TRIM31, CISH, SOCS5, TRIM39, RNF144B, FBXO40, KLHL6, FBXO11, GAN, FBXL14, FBXW5, RNF111, FBXL12, BTRC, or RNF126 or selected from FBXL12, FBXL14, FBXL15, KLHDC2, KLHL6, KBTBD7, ZER1, UBE2B or KLHL40. In some embodiments, the proximity effector polypeptide is selected from ZER1 FBXL12, FBXL14, FBXL15, KLHDC2, KLHL6, KBTBD7, UBE2B or KLHL40. In some embodiments, the proximity effector polypeptide is selected from UBE2B, UBE2A, ZER1, FBXL12, FBXL14, FBXL15, GABARAP, GABARAPL2, MAP1LC3A, KLHL6, KBTBD7, KLHDC2, KLHL40, KLHL40 fusion, KLHL6 fusion, or PRNP fusion. In some embodiments, the effector polypeptide is UBE2B, ZER1 KLHL40, KLHL41, DDI1, or PRPS2.

In some embodiments, the fusion polypeptide comprises a proximity effector polypeptide that is a degrader. In some embodiments, the proximity effector polypeptide is selected from GMCL1, FBXL15, PJA1, RNF115, DZIP3, RNF125, FBXO3, RNF185, RNF8, RNF183, RCHY1, KBTBD7, TRIM31, CISH, SOCS5, TRIM39, RNF144B, FBXO40, KLHL6, FBXO11, GAN, FBXL14, FBXW5, RNF111, FBXL12, BTRC, ZER1 or RNF126. In some embodiments, the proximity effector polypeptide is selected from FBXL12, FBXL14, FBXL15, KLHDC2, KLHL6, KBTBD7, ZER1 or, UBE2B. In some embodiments, the proximity effector polypeptide is a stabilizer, preferably KLHL40.

In some embodiments, the targeting moiety is a nanobody, ligand, interaction peptide or an antibody that binds the target polypeptide. In some embodiments, the targeting moiety is a nanobody, optionally vhhGFP or alpha-tag nanobody.

In some embodiments, the target polypeptide is selected from an oncogene polypeptide, oncogenic fusion polypeptide, synthetic lethal target, immunology/immune-oncology target, dominant gain-of-function disease variant, tumor suppressors, or unstable disease variant. In some embodiments, the oncogene polypeptide or oncogenic fusion polypeptide is RAS, MYC, or EWSR-FLI1. In some embodiments, the synthetic lethal target is EWSR1, SMARCA2/4, or PARP1. In some embodiments, the immunology/immune-oncology target is PD1/PD-L1 or JAK. In some embodiments, the dominant gain-of-function disease variant is FUS, TDP43, a-synuclein, amyloid beta precursor protein, HTT, or prion protein. In some embodiments, tumor suppressor is p53 or PTEN. In some embodiments, the unstable disease variant is a CFTR variant or dystrophin variant. In some embodiments, the target polypeptide is selected from EGFP-AB1, ABI1, Rluc, FUS S525L, NRAS, DNAJA3, BRAF, LAMP1, TDP43, Q311K, CD63, H2B, EGFR, DNAJB11 or WDR5.

Another aspect of the disclosure includes a fusion polypeptide described herein for use in making a medicament. In some embodiments, fusion polypeptide comprises the proximity effector KLHL40 or KLHL41 and a target polypeptide that is a loss of stability variant and the medicament is for treating muscular dystrophy. In some embodiments, the target polypeptide is BCR-Abl.

Another aspect of the disclosure includes a nucleic acid encoding any fusion polypeptide described herein. Another aspect of the disclosure includes a vector comprising any nucleic acid described herein.

### BRIEF DESCRIPTION OF THE DRAWINGS

An embodiment of the present disclosure will now be described in relation to the drawings in which:

FIG. 1A is a schematic of a method described herein.

FIG. 1B is a schematic of a method described herein.

FIG. 2 is an image depicting results of degradation and stabilization screens described herein.

FIG. 3A is a schematic depicting the structure of prion protein (PRNP) and FCGR3B.

FIG. 3B depicts schematics of the structure of recombinant and fusion PRNP and FCGR3B polypeptides and graphs depicting relative GFP intensity for each polypeptide.

FIG. 3C is a series of schematics depicting the amino acid sequences of recombinant FCGR3B polypeptides and graphs depicting relative GDP intensity of each polypeptide. SEQ ID NOs: 6-24 are shown.

FIG. 4A is a schematic of fusion polypeptides and a graph depicting relative fluorescence intensity of each polypeptide depicting degradation in trans.

FIG. 4B is a schematic of fusion polypeptides and a graph depicting relative fluorescence intensity of each polypeptide, depicting degradation in cis.

FIG. 5A is a graph depicting relative GFP intensity of Renilla, wild type UBE2B and mutant UBE2B comprising the mutation Cys88 to alanine.

FIG. 5B is a schematic of the structure of UBE2B and a graph depicting relative GFP intensity of Renilla, wild type UBE2B and mutants of UBE2B.

FIG. 5C is a graph depicting EGFP median fluorescence intensity of putative effector polypeptides.

FIG. 6A is a graph depicting results of a degradation assay involving EGFP and Renilla luciferase, KLHL40, DDI1, and PRPS2 with or without vhhGFP.

FIG. 6B is a series of schematics of the structure of DDI1 and recombinant DDI1 polypeptides and graph depicting relative GFP intensity of each polypeptide.

FIG. 6C is a graph depicting relative GFP intensity of PRS2 and mutant PRPS2.

FIG. 6D is a series of schematics of the structure of KLHL40 and recombinant KLHL40 polypeptides and a graph depicting relative GFP intensity of each polypeptide.

FIG. 6E is a series of schematics of the structure of KLHL40 and KLHL6 and recombinant polypeptides comprising domains from each of KLHL40 and KLHL6 and a graph depicting relative GFP intensity of each polypeptide.

FIG. 6F is an image depicting the amino acid sequences of a number of polypeptides. SEQ ID NOs: 25-39 are shown.

FIG. 7 is an image depicting results of an assay measuring activity of several polypeptides when tagged with either C-terminal or N-terminal vhhGFP.

FIG. 8 is an image depicting results a screen to identify putative effector polypeptides as degrader or stabilizer polypeptides of different target polypeptides.

FIG. 9 is a schematic of the structure target fusion polypeptides comprising unstable variants and GFP, a schematic of the structure of the putative effector polypeptide fused to vhhGFP and an image depicting activity of each effector on each target polypeptide.

FIG. 10A is an image depicting results of an assay measuring activity of effector polypeptides.

FIG. 10B is a graph depicting western blot quantification of effector polypeptides.

FIG. 10C is an image depicting results of assay measuring activity of effector polypeptides.

FIG. 11A is a schematic of the structure of vhhGFP fusion polypeptides used in a degradation and stabilization screen described herein.

FIG. 11B is an image depicting results of degradation and stabilization screens described herein.

FIG. 12A is a schematic of the structure of a GNMT H176N-GFP fusion polypeptide and vhhGFP fusion polypeptides used in a stabilization screen described herein.

FIG. 12B is an image depicting results of a stabilization screen described herein.

FIG. 13A is a graph depicting results of a stabilization assay described herein showing requirements for deubiquitinase function, in particular USP13.

FIG. 13B is a graph depicting results of a stabilization assay described herein showing requirements for deubiquitinase function, in particular USP38.

FIG. 13C is a graph depicting results of a stabilization assay described herein showing requirements for deubiquitinase function, in particular USP39.

FIG. 13D is a graph depicting results of a stabilization assay described herein showing requirements for deubiquitinase function, in particular OTUB1.

FIG. 14A depicts a schematic, image of western blot results, and graphs showing a graphical representation of band intensity of effector polypeptides described herein in the presence of absence of doxycycline.

FIG. 14B is a series of line graphs depicting relative proliferation over time of effectors described herein in the presence or absence of doxycycline.

FIG. 15A is a schematic and image of western blotting results for ARAF, effectors described herein, and Hsp90.

FIG. 15B depicts a schematic, image of western blot results for, and graphs showing a graphical representation of band intensity of effector polypeptides described herein in the presence of absence of doxycycline.

### DETAILED DESCRIPTION

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells. Generally, nomenclatures utilized in connection with, and techniques of, cell and tissue culture, molecular biology, and protein and oligonucleotide or polynucleotide chemistry and hybridization described herein are those well-known and commonly used in the art (see, e.g. Green and Sambrook, 2012).

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”. For ranges described herein, subranges are also contemplated, for example every, 0.1 increment there between. For example, if the range is 80% to about 90%, also contemplated are 80.1% to about 90%, 80% to about 89.9%, 80.1% to about 89.9% and the like.

The term “cell” as used herein refers to a single cell or a plurality of cells.

In understanding the scope of the present disclosure, the term “comprising” and its derivatives, (such as “comprise” and “comprises”), “having” (and any form of having, such as “have” and “has”), “including” (and any form of including, such as “include” and “includes”) or “containing” (and any form of containing, such as “contain” and “contains”), as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The definitions and embodiments described in particular sections are intended to be applicable to other embodiments herein described for which they are suitable as would be understood by a person skilled in the art.

As used herein, the terms “peptide,” “polypeptide,” and “protein” refer to any chain of two or more natural or unnatural amino acid residues, regardless of post-translational modifications (e.g., glycosylation or phosphorylation). Included are proteins that are a single polypeptide chain and multisubunit proteins (e.g. composed of 2 or more polypeptides).

The term “antibody” as used herein is intended to include monoclonal antibodies, polyclonal antibodies, single chain, humanized and other chimeric antibodies, or fully human antibodies, as well as binding fragments thereof, for example nanobodies. The antibody may be from recombinant sources and/or produced in transgenic animals. Also included are antibodies that can be produced through using biochemical techniques or isolated from a library.

As used herein, the term “putative proximity effector polypeptide” or “putative effector polypeptide” means as used herein a polypeptide that is to be screened, for example in an assay or method described herein, for ability to degrade or stabilize a target polypeptide, to cause cell death in a cell comprising the target polypeptide, or to promote membrane localization of cell surface polypeptides. For example, the ORFs of an ORFeome library expressed in a cell are putative proximity effector polypeptides.

As used herein, the term “proximity effector polypeptide” or “effector polypeptide” means as used herein a polypeptide that is able to induce a biological effect when in proximity of a target polypeptide, for example, degrade or stabilize a target polypeptide, able to cause cell death in a cell comprising the target polypeptide, or able to promote membrane localization of cell surface polypeptides.

The term “effector” can be used to refer to a putative proximity effector polypeptide or a proximity effector polypeptide or both.

The term “fluorescent polypeptide” as used herein refers to fluorescent polypeptides that can be appended or introduced into a peptide, antibody or other compound described herein and which is capable of producing, either directly or indirectly, a detectable fluorescent signal.

As used herein, the term “target polypeptide” means as used herein a polypeptide of interest which is to be targeted in a proximity-induced interaction, for example with a putative proximity effector polypeptide that is to be screened, for example in an assay or method described herein, to determine whether it is a proximity effector, e.g., degrades or stabilizes the target polypeptide, or for example with a degrader polypeptide or stabilizer polypeptide where the target polypeptide is to be degraded or stabilized. The target polypeptide may be brought into proximity with another polypeptide using for example, a targeting moiety fused to the effector polypeptide, for example a degrader polypeptide, or a stabilizer polypeptide, which binds to the target polypeptide. The target polypeptide may be in a fusion polypeptide comprising a peptide interaction tag such as ABI1, for example EGFP-ABI1. The target polypeptide can be a multi-subunit protein. The target polypeptide can be any species, including mammalian, preferably human. The target polypeptide can be an endogenous polypeptide or a recombinantly expressed polypeptide.

As used herein, the term “degrader polypeptide” which may simply be referred to as “degrader” means a polypeptide which, when in proximity to a target polypeptide in a cell, degrades or results in degradation of the target polypeptide by at least 10 percent for example as compared to a control. A degrader polypeptide may be brought into proximity of the target polypeptide using for example, a targeting moiety fused to the degrader polypeptide which binds to the target polypeptide, directly or indirectly for example using SpyTag/SpyCatcher, SnoopTag/SnoopCatcher, HiBiT/LgBit, or GFP11/GFP1-10 systems. The degrader polypeptide may decrease the half-life of the target polypeptide and/or may decrease the level of a target polypeptide in a cell. Depending on the context, reference to degrader polypeptide or degrader can also refer to the nucleic acid sequence encoding the degrader polypeptide.

As used herein, the term “stabilizer polypeptide” or which may simply be referred to as “stabilizer” means a polypeptide which, when in proximity to a target polypeptide in a cell, stabilizes or results in stabilization of the target polypeptide. A stabilizer polypeptide may be brought into proximity of the target polypeptide using for example, a targeting moiety fused to the stabilizer polypeptide which binds to the target polypeptide. The stabilizer polypeptide may increase the half-life of the target polypeptide and/or may increase the level of the target polypeptide in a cell for example as compared to a control. Depending on the context, reference to stabilizer polypeptide or stabilizer can also refer to the nucleic acid sequence encoding the degrader polypeptide.

As used herein, a “lethal polypeptide” includes a polypeptide that when brought into proximity to a target polypeptide, causes cell death.

As used herein, the term “protein trafficking polypeptide” includes polypeptide which promote membrane localization of cell surface polypeptides, including mutant cell surface polypeptides.

As used herein, the term “targeting moiety” includes a polypeptide, for example, an antibody, antibody binding fragment, nanobody, ligand or interaction peptide optionally of a fusion polypeptide, which binds the target polypeptide. The targeting moiety can be fused to the effector, e.g., degrader or stabilizer, polypeptide. The targeting moiety may in some cases bring the putative proximity effector polypeptide or proximity effector polypeptide into proximity constitutively, for example when the targeting moiety is a binding protein such as an antibody, optionally a nanobody. The targeting moiety may in some cases only bind the target polypeptide upon induction, for example through chemical dimerization in the presence of another molecule, for example a chemical inducer, such as abscisic acid (e.g., for example leading to interaction of proteins comprising interacting ABI1 and PYL1 domains). Such chemical inducers and the domains in which they will induce chemical dimerization are described herein and known in the art for example as described in Ziegler et al., Mandipropamid as a chemical inducer of proximity for in vivo applications. Nat Chem Biol 18, 64-69 (2022), which is herein incorporated by reference. The targeting moiety can be any molecule that binds to the target polypeptide with a k_{d} of lower than 10e⁻⁴M in a binding affinity assay, optionally by SPR.

The term “interaction peptide” as used herein can include for example a peptide that specifically interacts or can be induced to interact or dimeraize with another interaction peptide (which can be referred to as a complementary interaction peptide). Examples of interaction peptides (and complementary interaction peptides) include but are not limited to ABI1 and PYL1, ABI1 and PYR, GID1 and GAI or FKBP and FRB which bind each other in the presence of a chemical inducer. It is to be understood that examples of interaction peptide are also examples of complementary interaction peptides.

As used herein, the term “negative selection marker” includes selectable markers that eliminate or inhibit growth of the host organism upon selection. An example of a negative selection marker is thymidine kinase, in the presence of ganciclovir, or engineered deoxycytidine kinase (DCK*) in the presence of 2-bromovinyldeoxyuridine or L-deoxythymidine.

As used herein, the term “synthetic lethal target” includes polypeptides that when inhibited or activated cause cell death only under certain conditions. For example, SMARCA2 is a synthetic lethal target when present in SMARCA4 mutant tumors.

An aspect of the disclosure includes a fusion polypeptide comprising an, or at least one, proximity effector polypeptide selected from Table 4 and a or at least one, targeting moiety that binds the, or at least one, target polypeptide. In some embodiments, the, or the at least one, proximity effector polypeptide is selected from Table 5 or Table 6 or Table 7.

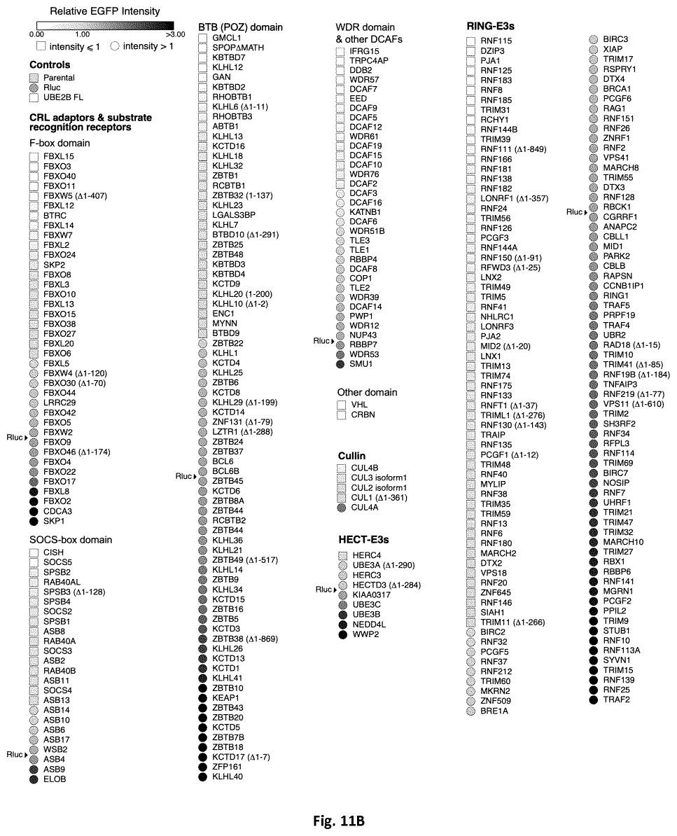

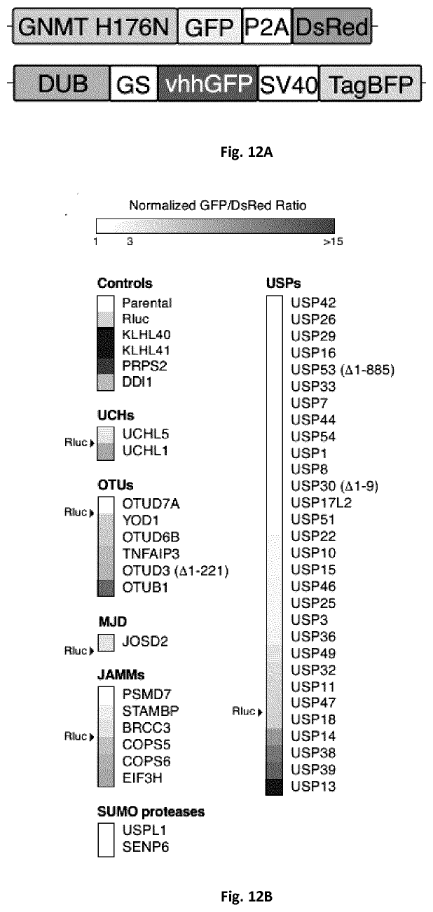

In some embodiments, the, or at least one proximity effector polypeptide is selected from those in FIG. 12B. In some embodiments, the, or the at least one, proximity effector polypeptide is selected from those in FIG. 11B.

In some embodiments, the, or the at least one, proximity effector polypeptide is selected from UBE2B, UBE2A, FBXL12, FBXL14, FBXL15, GABARAP, GABARAPL2, MAP1LC3A, KLHL6, KBTBD7, KLHDC2, KLHL40 (optionally a KLHL40 fragment), KLHL40 fusion, ZER1 or PRNP fusion.

In an embodiment, the KLHL40 is a KLHL40 fragment, for example a fragment listed in Table 4.

In some embodiments, the or the at least one proximity effector polypeptide is selected from UBE2B, FBXL12, FBXL14, FBXL15, KLHL6, KBTBD7, KLHDC2, KLHL40, KLHL40 fusion, or KLHL6. As indicated in the Examples, many of the proximity effectors identified had activity that was greater than known proximity degraders CRBN and VHL.

Accordingly, in an embodiment the, or least one proximity effector polypeptide is selected from GMCL1, FBXL15, PJA1, RNF115, DZIP3, RNF125, FBXO3, RNF185, RNF8, RNF183, RCHY1, KBTBD7, TRIM31, CISH, SOCS5, TRIM39, RNF144B, FBXO40, KLHL6, FBXO11, GAN, FBXL14, FBXW5, RNF111, FBXL12, BTRC, or RNF126.

In some embodiments, the or the at least one effector polypeptide is selected from UBE2B, KLHL40, KLHL41, DDI1, or PRPS2. In some embodiments, the, or the at least one targeting moiety is a nanobody, ligand, interaction peptide or an antibody that binds the at least one target polypeptide. The targeting moiety can bind an endogenous target polypeptide directly or indirectly. The targeting moiety can also bind a tag or interaction peptide that has been fused to the endogenous target polypeptide. In some embodiments, the or at least one targeting moiety is a nanobody, optionally vhhGFP or epitope tag binding protein such as a binding protein (antibody or nanobody) to ALFA-tag, myc tag, Flag tag, HA tag or V5 tag. Where the targeting moiety is or comprises an interaction peptide, the effector can also comprise an interaction peptide that is complementary to the targeting moiety interaction peptide.

In some embodiments, the or the at least one target polypeptide is selected from an oncogenic polypeptide, oncogenic fusion protein, synthetic lethal target, immunology/immune-oncology target, dominant gain-of-function disease variant, tumor suppressor protein, or unstable disease variant.

In some embodiments, the oncogenic polypeptide or oncogenic fusion protein is RAS, MYC, or EWSR-FLI1.

In some embodiments, the synthetic lethal target is EWSR1, SMARCA2/4, or PARP1. In some embodiments, the synthetic lethal target is SMARCA2 in the presence of SMARCA4 mutants, SMARCA4 in the presence of SMARCA2 mutants, PARP1 in BRCA1/2 deficient cells, and/or PRMT5 in cells with a loss of MTAP.

An immunology/immune-oncology target includes for example proteins involved in T cell mediated killing of tumor cells. In some embodiments the immunology/immune-oncology target is PD1/PD-L1 or JAK.

In some embodiments, the dominant gain-of-function disease variant is a FUS, TDP43, a-synuclein, amyloid beta precursor protein, HTT, or a prion protein gain of function disease variant. In some embodiments, the dominant gain-of-function disease variant is selected from Table 1. Dominant gain of function disease variants include proteins with a mutation which leads to a gain of function (i.e. toxicity) causing a disease phenotype.

TABLE 1
Examples of dominant gain-of-function disease variants

| | | amyloid beta | | | Prion | |
|---|---|---|---|---|---|---|
| TARDBP | a-synuclein | precursor | | | protein | |
| (TDP43) | (SNCA) | (APP) | TTR | HTT | (PRNP) | FUS |
| P112H | A53T | KM670- | C30R | Poly-Q | D178N | G191S |
| | | 671NL | | expansions | | |
| D169G | A30P | D678N | L32P | | V180I | R216C |
| K263E | E46K | A692G | D38E | | E196K | G225V |
| N267S | H50Q | E693G | D38G | | E200K | G230C |
| G287S | G51D | A713T | V40I | | V203I | R234C |
| G290A | | T714A | S43N | | R208H | R244C |
| G294V | | T714I | P44S | | V210I | R495X |
| G294A | | V715M | V48M | | E211Q | G507D |
| G295S | | I716V | V50A | | M232R | R514G |
| G295R | | V717F | V50G | | | R514S |
| G295C | | V717G | V50L | | | G515C |
| G298S | | V717I | V50M | | | R518K |
| M311V | | V717L | F53I | | | R521C |
| A315E | | L723P | F53L | | | R521G |
| A315T | | E693K | F53V | | | R521H |
| A321V | | E693Q | R54T | | | R522G |
| A321G | | D594N | K55N | | | R524S |
| Q331K | | L705V | A56P | | | R524T |
| S332N | | | D58A | | | P525L |
| G335D | | | D58V | | | Y526YY |
| M337V | | | W61L | | | |
| Q343R | | | E62D | | | |
| N345K | | | E62G | | | |
| G348C | | | F64S | | | |
| G348V | | | A65D | | | |
| G348R | | | A65S | | | |
| N352S | | | A65T | | | |
| N352T | | | G67A | | | |
| G357S | | | G67E | | | |
| G357R | | | G67R | | | |
| M359V | | | G67V | | | |
| R361S | | | T69A | | | |
| R361T | | | T69I | | | |
| P363A | | | S70I | | | |
| G368S | | | S70R | | | |
| Y374X | | | S72P | | | |
| G376D | | | G73E | | | |
| N378D | | | E74G | | | |
| N378S | | | E74K | | | |
| S379C | | | L75P | | | |
| S379P | | | L75Q | | | |
| A382T | | | L78H | | | |
| A382P | | | L78R | | | |
| I383V | | | T79K | | | |
| G384R | | | T80A | | | |
| W385G | | | E81G | | | |
| S387delins TNP | | | E81K | | | |
| N390D | | | F84L | | | |
| N390S | | | I88L | | | |
| S393L | | | Y89H | | | |
| | | | K90N | | | |
| | | | V91A | | | |
| | | | I93V | | | |
| | | | S97Y | | | |
| | | | Y98F | | | |
| | | | I104N | | | |
| | | | I104S | | | |
| | | | I104T | | | |
| | | | E109K | | | |
| | | | E109Q | | | |
| | | | A111S | | | |
| | | | A117G | | | |
| | | | A117S | | | |
| | | | T126N | | | |
| | | | I127M | | | |
| | | | I127V | | | |
| | | | L131M | | | |
| | | | Y134C | | | |
| | | | Y136S | | | |
| | | | A140S | | | |
| | | | V142A | | | |
| | | | V142I | | | |
| | | | N144S | | | |

In some embodiments, the tumor suppressor protein is p53 or PTEN.

An unstable disease variant includes a protein with a mutation that leads to misfolding and/or degradation of the protein, causing a disease phenotype. In some embodiments the unstable disease variant is a CFTR variant or dystrophin variant. In some embodiments, the unstable disease variant is CFTR delta508, ACTB E364K, ALDOA E206K, AMHR2 R54C, AMPD3 A320V, CBS L456P, GNMT H176N, PIKLR F132L, SCARB H363N, or TPMT A80P.

In some embodiments, the or the at least one target polypeptide is selected from EGFP-ABI1, Rluc, FUS S525L, NRAS, DNAJA3, BRAF, LAMP1, TDP43 Q311K, CD63, H2B, EGFR, DNAJB11 or WDR5.

In some embodiments, the at least one target polypeptide is a human polypeptide.

The fusion polypeptide can comprise a proximity effector selected from any group or be a subgroup of any group described herein which can be combined with any targeting moiety or group of targeting moieties described herein.

The fusion polypeptide can comprise the or the at least one proximity effector polypeptide selected from Table 4, 5, 6 or 7 C-terminal to the or the at least one targeting moiety that binds the, or the at least one, target polypeptide and/or N-terminal to the or the at least one targeting moiety that binds the, or the at least one, target polypeptide. For example, PRR20A, MYLIP, KLHL22, MAP1LC3A, GABARAPL2, GABARAP, FBXL15, TRIM39, NHLRC1, MAP1LC3B, KLHL6, DCAF15, KLHDC2, RNF166, RTL8C, SPOP, LY6D, ASB6, or PRNP can be C-terminal to the, or the at least one, targeting moiety that binds the, or the at least one, target polypeptide.

Reference to a proximity effector includes reference to its active fragments. For example, as shown herein different KLHL40 fragments that include a BTB domain act as a stabilizing effector. KLHL40 fragments that are deleted for BTB domain act as a degrading effector. Reference to KLHL40 can refer to the full protein, for example the sequence provided in accession number Q2TBA0 or an active fragment thereof. Reference to the KLHL40 fusion for example, refers to KLHL40 where the BTB domain of KLHL40 is swapped with the BTB domain of KLHL6. Effector fusions that can be included in fusion polypeptides are described herein for example in Tables 4, 5, 6 and 7 and in the Examples. Reference to such fusions include for example the portions described in the Tables.

The fusion polypeptides can comprise linkers, linking the or the at least one effector and the or the at least target moiety e.g., nanobody. Different linkers can be used. Short and long linkers were assessed with different effectors. Linker length did not impact the effectiveness of the effector in assays where the effector targeting moiety fusion polypeptide was expressed with a target polypeptide.

Another aspect of the disclosure includes a nucleic acid encoding any fusion polypeptide described herein.

Another aspect of the disclosure includes a nucleic acid encoding: a or at least one target polypeptide optionally comprising or selected from EGFP-AB1, Rluc, FUS, optionally FUS S525L, NRAS, DNAJA3, BRAF, LAMP1, TDP43, optionally TDP43 Q311K, CD63, H2B, EGFR, DNAJB11, WDR5, RAS, MYC, or EWSR-FLI1, EWSR1, SMARCA2/4, or PARP1, PD1/PD-L1, JAK, a-synuclein, amyloid beta precursor protein, HTT, prion protein, p53, PTEN, a CFTR variant, or dystrophin variant; a first fluorescent polypeptide, and an IRES or cleavage site therebetween (e.g. between the target polypeptide and the fluorescent protein), optionally for use in a method, process, assay, kit or as otherwise described herein.

Another aspect of the disclosure is a kit comprising one or more components described herein. In one embodiment, the kit is for use in a method described herein. In an embodiment, the kit comprises any of the nucleic acids described herein. In some embodiments, the kit comprises any of the fusion polypeptides described herein. In an embodiment, the kit comprises a cell line described herein. The nucleic acid may be comprised in a vector, including a vector described herein. The kit in some embodiments, comprises a fusion polypeptide described herein. In some embodiments, the kit further comprises a vial or other housing comprising for example, the nucleic acid or fusion polypeptide. In some embodiments, the kit further comprises a set of instructions, or one or more reagents for performing an assay descried herein. In some embodiments, the kit comprises any library described herein, optionally an ORFeome library comprising at least one putative effector polypeptide fused to a targeting moiety that binds to the at least one target polypeptide.

In some embodiments, the at least one target polypeptide is fused to a second fluorescent polypeptide.

In some embodiments, the first fluorescent polypeptide is RFP, YFP, mCherry, mCitrine, mNeonGreen, mScarlet, BFP or GFP.

In some embodiments, the second fluorescent polypeptide is RFP, YFP, mCherry, mCitrine, mNeonGreen, mScarlet, BFP or GFP and the fluorescent signal emitted by the first and the second fluorescent polypeptides is distinguishable using flow cytometry.

In some embodiments, the at least one effector polypeptide is a plurality of effector polypeptides.

The fusion polypeptides can be used for making a medicament. As mentioned herein, fusions comprising KLHL40 may be particularly useful for targeting unstable variants found in muscular dystrophy. Also, it was demonstrated herein that BCR-Abl, a fusion protein involved in leukemia, can be targeted by effectors described herein.

Another aspect of the disclosure is vector comprising any nucleic acid described herein.

A further aspect includes a recombinant cell comprising a nucleic acid or expressing a fusion protein described herein.

The fusion polypeptides, nucleic acids, vectors, kits, uses and cells can be used in one or more methods, processes, or assays described herein.

Provided herein are non-biased approaches to conducting proximity screens. As demonstrated, the methods employed identified a number of proximity effectors which can degrade or stabilize a target polypeptide. Such methods can be employed to identify other effectors in addition to stabilizers and/or degraders.

An aspect includes a method of identifying a proximity effector polypeptide, the method comprising:
transducing an ORFeome library into a plurality of cells, the ORFeome library encoding a plurality of ORFs, wherein each of the ORFs is fused to a targeting moiety that binds or can be induced to bind to the target polypeptide directly or indirectly;
expressing the plurality of ORFs of the ORFeome library in the transduced plurality of cells, under conditions for the targeting moiety to interact with the target polypeptide, wherein; and
determining whether any of the plurality of ORFs is a proximity effector polypeptide by measuring abundance, optionally total abundance, cell surface abundance or subcellular abundance, of the target polypeptide in cells expressing any of the plurality of ORFs compared to a control and/or detecting whether any of the plurality of ORFs is depleted or enhanced in the transduced plurality of cells compared to a control;
wherein the transduced plurality of cells recombinantly expresses the target polypeptide and optionally expresses a first fluorescent polypeptide, wherein the target polypeptide is a second fluorescent polypeptide, endogenous protein, or a fusion polypeptide fused to a second fluorescent polypeptide epitope tag, an antibiotic resistance protein and/or a negative selection marker; and
wherein an ORF encodes a proximity effector polypeptide when the ORF increases or decreases the target polypeptide abundance compared to control, or is depleted or enhanced in the transduced plurality of cells compared to the control.

The first fluorescent polypeptide can act as an internal normalization and can increase sensitivity of screens. In some embodiments, the plurality of cells expresses a first fluorescent polypeptide.

In particular, the methods can be used to identify stabilizers and degraders.

Accordingly, another aspect of the disclosure includes a method of identifying a or at least one putative effector polypeptide as a degrader or stabilizer of a target polypeptide, the method comprising:
transducing an ORFeome library into a plurality of cells,
expressing the or the at least one putative effector polypeptide of the ORFeome library, the at least one putative effector polypeptide fused to a targeting moiety that binds to the at least one target polypeptide, and
determining whether the level of the at least one target polypeptide in the at least one cell has been decreased or has increased relative to a control,
   wherein the plurality of cells each recombinantly express a first fluorescent polypeptide and the at least one target polypeptide that is fused to a second fluorescent polypeptide, or wherein the target polypeptide is fused to an antibiotic resistance protein or negative selection marker, and
   wherein, the at least one putative effector polypeptide is a degrader of the target polypeptide when the level of the target polypeptide is decreased and the at least one effector polypeptide is a stabilizer of the target polypeptide when the level of the target polypeptide has increased.

Measuring the abundance or level of a target polypeptide can comprise determining whether the target polypeptide has decreased or has increased relative to a control, or whether cell surface level of the target polypeptide has increased or decreased relative to control or whether a particular subcellular fraction or organelle level of the target organelle has increased or decreased relative to control. Various methods can be used to assess polypeptide levels depending on the combination of sensors and tags used and the type of proximity effector identified. For example, as demonstrated in the example, when fluorescent tags, the level of fluorescence of a desired fraction can be measured and compared to unsorted cells.

Alternatively, in some embodiments, the determining may comprise monitoring the level of the or the at least one target polypeptide with an antibody specific to the target polypeptide. For example, when fluorescent tags are not used, immunoaffinity techniques using tagged binding proteins such as fluorescently labelled antibodies or immunomagnetic beads that directly or indirectly detect a target polypeptide such as a cell surface target polypeptide and can be separated by FACS or magnetic separation.

The increase or decrease may for example be at least 10 percent or for example, “decreased” can refer to a statistically significant decrease, for example where p<0.05, relative to a control or for example “increased” can refer to a statistically significant increase, for example where p<0.05, relative to a control.

The control can for example be when using fluorescence, unsorted cells or when using antibiotic resistance or negative selection untreated cells.

In another embodiment, the targeting moiety is a nanobody, ligand or an antibody that binds the target polypeptide. In some embodiments, the targeting moiety is any molecule that binds to the at least one target polypeptide with a k_{d} of lower than 10e⁻⁴ for example, 10e⁻⁵ or 10e⁻⁶. In some embodiments, the targeting moiety is a HaloTag™ (haloalkane dehalogenase). For example, a HaloTag™ could be used as a targeting moiety where the at least one target polypeptide has a known ligand and a chloroalkane derivative of such a ligand is synthesized. For example, where the target polypeptide is BRD4, the ligand could be JQ1-chloroalkane which binds BRD4, and the effector polypeptide would be fused to the HaloTag. In this example, BRD4 levels could be followed with fluorescence-activated cell sorting (FACS) using a BRD4 specific antibody coupled to a fluorophore. Alternatively, one could use cell viability as a readout, since BRD4 is an essential gene in many cell lines. using cell viability as a readout, where degradation of BRD4 is identified by cell death. In some embodiments, the control includes a cell (e.g., cell line) expressing the at least one target polypeptide that has not been transduced with the ORFeome library or the at least one putative effector polypeptide. In some embodiments, the control is a cell that does not express any effectors. In some embodiments, the control is a cell that expresses an inert putative effector (for example, luciferase) coupled to a targeting moiety. In some embodiments, genetic constructs, such as fusion proteins comprising for example, antibodies, nanobodies or other targeting moieties, could be delivered through nucleic acids encoded in viral vectors, such as AAV, adenovirus, herpesvirus vectors or using liposomes or lipid nanoparticles.

In some embodiments, the method further comprises generating a cell line expressing a target polypeptide that is or is fused to a second fluorescent polypeptide, and/or is fused to epitope tag, an antibiotic resistance protein and/or a negative selection marker. The target polypeptide can also be an unlabelled or untagged polypeptide (e.g., an endogenous polypeptide recombinantly expressed). In some embodiments, the cell line further comprises a first fluorescent polypeptide. The cell line can be produced using for example HEK293 cells, 293T cells, HeLa cells, HCT116 cells, SH-SY5Y cells, Hap1 cells, HepG2 cells, MiaPaCa cells, A549 cells, THP-1 cells, Jurkat cells, or K562 cells. In an embodiment, the cell line is a cell line disclosed herein. In some embodiments, the generating of the cell line comprises introducing a nucleic acid encoding a target polypeptide, optionally any of the nucleic acids disclosed herein or nucleic acids encoding polypeptides described herein, into the cell and selecting stably transduced cells and producing a clonal cell line. The method can include selecting a clone where the target polypeptide is expressed at a desired level. The method can include a step described in the Examples. In some embodiments, the nucleic acid encodes a target polypeptide and optionally a first fluorescent polypeptide, optionally wherein the target polypeptide is fused to a second fluorescent polypeptide or an antibiotic resistance protein or a negative selection marker. In some embodiments, the nucleic acid encodes a target polypeptide selected from EGFP-AB1, Rluc, FUS S525L, NRAS, DNAJA3, BRAF, LAMP1, TDP43 Q311K, CD63, H2B, EGFR, DNAJB11, WDR5, RAS, MYC, or EWSR-FLI1, EWSR1, SMARCA2/4, or PARP1, PD1/PD-L1, JAK, FUS, TDP43, a-synuclein, amyloid beta precursor protein, HTT, prion protein, p53, PTEN, a CFTR variant, or dystrophin variant; a first fluorescent polypeptide, and an IRES. In some embodiments, the cell line is a 293T cell line, optionally expressing an ABI1-GFP fusion. In other embodiments, any cell line can be used.

In some embodiments, the decrease or increase in the level of the target polypeptide is determined by calculating a ratio of the second fluorescent polypeptide to the first fluorescent polypeptide using flow cytometry. In some embodiments, the fluorescent polypeptide is any fluorescent protein known in the art for example those which are disclosed in public database fpbase.orr. In some embodiments the first or second fluorescent polypeptide is RFP, YFP, mCherry, mCitrine, mNeonGreen, mScarlet, BFP, GFP, or any variant thereof (for example EGFP). In one embodiment, the first fluorescent polypeptide is GFP, and the second fluorescent polypeptide is BFP.

For methods, processes, screening assays etc. involving the target polypeptide fused to an antibiotic resistance polypeptide or negative selection marker, the proximity effector can be a degrader or a stabilizer or other effector. For example, after isolating surviving cells the transduced plurality of cells treated with antibiotic or negative selection drug, can be assessed for the abundance of each ORF (e.g., putative proximity effector). ORFs that are depleted in the transduced plurality of cells compared to control (e.g., unselected cells) indicates that those ORFs are degrader(s) effectors and ORFs that are enriched in the transduced plurality of cells compared to control indicates that those ORFs are stabilizers.

Accordingly, in some embodiments, the target polypeptide is fused to an antibiotic resistance protein or negative selection marker. In some embodiments, a decrease or increase in the level of the target polypeptide as compared to a control is determined by measuring the relative abundance of each ORF in surviving cells of the plurality of transduced cells compared to control. In some embodiments, the antibiotic resistance protein is puromycin acetyltransferase and the method further comprises adding puromycin to the plurality of transduced cells. In some embodiments, the survival of a cell of the transduced plurality of cells when exposed to puromycin can indicate the level of the target polypeptide was increased as compared to a control and/or the death of a cell of the transduced plurality of cells when exposed to puromycin can indicate that the level of the target polypeptide is decreased as compared to a control. In some embodiments, the increase in growth of cells of the transduced plurality of cells as compared to a control when exposed to puromycin indicates the level of the target polypeptide was increased as compared to a control (e.g. the effector is a stabilizer) and a lesser degree of growth of a cell of the transduced plurality of cells as compared to a control when exposed to puromycin indicates that the level of the target polypeptide is decreased as compared to a control (e.g. the effector is a degrader). Other antibiotic resistance proteins can also be used, for example neomycin phosphotransferase, blasticidin deaminase, or hygromycin kinase. The antibiotic used would be for example, neomycin, blasticidin or hygromycin respectively.

It is understood that depending on the negative selection marker being used, the growth, lack of growth, or death of the cell could be indicative of an increase or decrease as compared to a control. In some embodiments the negative selection marker is thymidine kinase and the method further comprises adding ganciclovir to the transduced plurality of cells. In some embodiments, the survival of a cell of the plurality of the cells when exposed to ganciclovir indicates that the level of the target polypeptide is decreased as compared to a control and the death of a cell of the transduced plurality of cells when exposed to ganciclovir indicates that the level of target polypeptide was increased as compared to a control. In some embodiments, the increase in growth of a cell as compared to a control of the plurality of the cells when exposed to ganciclovir indicates that the level of the target polypeptide is decreased as compared to a control and a lesser degree of growth of a cell of the transduced plurality of cells as compared to a control when exposed to ganciclovir indicates that the level of target polypeptide was increased as compared to a control.

In some embodiments, the determining comprises assessing proliferation of the transduced plurality of cells and ORF(s) identified in a cell of the transduced plurality of cells that enhance(s) or decrease(s) cell proliferation compared to a control is/are a proximity effector polypeptide. Proliferation can be assessed by measuring the relative abundance of each ORF in the transduced plurality of cells after selection compared to before selection (e.g. control). ORFs that promote growth would be enriched, whereas ORFs that inhibit growth would be depleted.

In some embodiments, the plurality of cells is transduced to maintain about on average >300, >400 or about on average >500 fold coverage of the ORFeome library.

In some embodiments, the control includes a cell (e.g., cell line) expressing the at least one target polypeptide, that has not been transduced with the ORFeome library or the at least one putative effector polypeptide.

In some embodiments, the method, process or screening assay further comprises expressing at least one of the effector polypeptides identified as stabilizers or degraders in the above-mentioned methods in at least one cell expressing the target polypeptide, optionally wherein the target polypeptide is in a fusion polypeptide, wherein the at least one effector polypeptide is fused to a targeting moiety, and determining whether the level of the target polypeptide in the at least one cell has been decreased or has increased relative to a control. For example, these additional steps may be used to validate that effector polypeptides identified as degraders or stabilizers of the target polypeptide in the above-mentioned methods, processes and screening assays do degrade or stabilize the target polypeptide. In some embodiments, the control is a cell or plurality of cells expressing the target polypeptide that has not been transduced with the effector polypeptide, has not been induced for example by chemical dimerization, is unsorted and/or has not been subjected to selection (e.g., antibiotic or negative selection).

In some embodiments, the library is an ORFeome-derived lentiviral pooled library. Many different kinds of libraries are well known in the art and can be used in the methods and processes of the present disclosure. Examples of libraries that may be used in the present disclosure include synthetic libraries of viral or bacterial proteins, protein domain libraries (from human proteins or other proteomes), fragment libraries (from human proteins or other proteomes) or e.g., using insertion mutagenesis to insert the proximity-inducing tag randomly to the genome with a splice acceptor sequence to fuse it to gene fragments.

In some embodiments, the targeting moiety brings the putative proximity effector or proximity effector polypeptide into proximity with the target polypeptide via chimerical dimerization and the method further comprises administering a chemical inducer. In some embodiments, the effector polypeptide is fused to the targeting moiety and the targeting moiety is a nanobody. Other affinity binding agents such as single chain antibodies can also be used.

In some embodiments, the target polypeptide is in a fusion polypeptide comprising ABI1. In some embodiments, the putative effector polypeptide or effector polypeptide is a fusion polypeptide fused to a targeting moiety, wherein the targeting moiety is PYL1. For example, PYL1 can bind ABI1 in the presence of abscisic acid. In some embodiments, the target polypeptide is in a fusion polypeptide comprising ABI1 and the targeting moiety is PYL1, and the method comprises administering abscisic acid as a chemical inducer.

In some embodiments, the target polypeptide is in a fusion polypeptide comprising FKBP and the putative effector polypeptide or effector polypeptide is a fusion polypeptide fused to a targeting moiety, wherein the targeting moiety is FRB, wherein for example, FKBP and FRB bind in the presence of rapamycin. In some embodiments, the target polypeptide is in a fusion polypeptide comprising FKBP and the targeting moiety is FRB, and the method comprises administering rapamycin as a chemical inducer.

In some embodiments, the target polypeptide is in a fusion polypeptide comprising FRB, and the putative effector polypeptide or effector polypeptide is in a fusion polypeptide fused to a targeting moiety, wherein the targeting moiety is FKRB, wherein for example, FKBP and FRB bind in the presence of rapamycin. In some embodiments, the target polypeptide is in a fusion polypeptide comprising FRB and the targeting moiety is FKBP and the method comprises administering rapamycin as a chemical inducer.

In some embodiments, the target polypeptide is in a fusion polypeptide comprising FKBP, and the putative effector polypeptide or effector polypeptide is in a fusion polypeptide fused to a targeting moiety, wherein the targeting moiety is mutant FRB, wherein for example, FKBP and mutant FRB bind in the presence of rapalogs such as AP21967. In some embodiments, the target polypeptide is in a fusion polypeptide comprising FKBP and the targeting moiety is FRB, and the method comprises administering AP21967 as a chemical inducer.

In some embodiments, the target polypeptide is in a fusion polypeptide comprising mutant FRB, and the putative effector polypeptide or effector polypeptide is in a fusion polypeptide fused to a targeting moiety, wherein the targeting moiety is FKBP, wherein for example, FKBP and mutant FRB bind in the presence of rapalogs such as AP21967. In some embodiments, the target polypeptide is in a fusion polypeptide comprising FRB and the targeting moiety is FKBP and the method comprises administering AP21967 as a chemical inducer.

In some embodiments, the target polypeptide is in a fusion polypeptide comprising GID1, and the putative effector polypeptide or effector polypeptide is in a fusion polypeptide fused to a targeting moiety, wherein the targeting moiety is GAI, wherein for example, Gibberellin insensitive dwarf 1 (GID1) and GA insensitive (GAI) bind in the presence of gibberellic acid. In some embodiments, the target polypeptide is in a fusion polypeptide comprising GID1 and the targeting moiety is GAI, and the method comprises administering gibberellic acid as a chemical inducer.

In some embodiments, the target polypeptide is in a fusion polypeptide comprising GAI, and the putative effector polypeptide or effector polypeptide is in a fusion polypeptide fused to a targeting moiety, wherein the targeting moiety is GID1, wherein for example, GID1 and GAI bind in the presence of gibberellic acid. In some embodiments, the target polypeptide is in a fusion polypeptide comprising GAI and the targeting moiety is GID1 and the method comprises administering gibberellic acid as a chemical inducer.

In some embodiments, the target polypeptide is in a fusion polypeptide comprising ABI1, and the putative effector polypeptide or effector polypeptide is in a fusion polypeptide fused to a targeting moiety, wherein the targeting moiety is PYR1, wherein for example, ABI1 and PYR1 bind in the presence of mandipropamid. In some embodiments, the target polypeptide is in a fusion polypeptide comprising ABI1 and the targeting moiety is PYR1, and the method comprises administering mandipropamid as a chemical inducer.

In some embodiments, the target polypeptide is in a fusion polypeptide comprising PYR1, and the putative effector polypeptide or effector polypeptide is a fusion polypeptide fused to a targeting moiety, wherein the targeting moiety is ABI1, wherein for example, ABI1 and PYR1 bind in the presence of mandipropamid. In some embodiments, the target polypeptide is in a fusion polypeptide comprising PYR1 and the targeting moiety is ABI1, and the method comprises administering mandipropamid as a chemical inducer.

Also provided in another aspect is a process for modulating a target polypeptide in at least one cell, the method comprising: expressing the proximity effector polypeptide provided in Table 4, 5, or 7 in the at least one cell, the at least one proximity effector polypeptide fused to a targeting moiety. The process can be for targeted degradation or targeted stabilization.

For example, in some embodiments, the process is for targeted degradation of at least one target polypeptide in at least one cell, the method comprising expressing at least one degrader provided in Table 4 in the at least one cell, the at least one degrader polypeptide fused to a targeting moiety. In some embodiments, the at least one degrader is provided in Table 5, 6 or 7. In some embodiments, the at least one degrader is provided in FIG. 11B.

In some embodiments, the at least one target polypeptide is an oncogene polypeptide or oncogenic fusion protein, optionally RAS, MYC, and/or EWSR-FLI1 and the degrader polypeptide is selected from UBE2B, UBE2A, FBXL12, FBXL14, FBXL15, GABARAP, GABARAPL2, MAP1LC3A, KLHL6, KBTBD7, PRR20A or KLHDC2.

In some embodiments, the at least one target polypeptide is a synthetic lethal target, optionally SMARCA2 in the presence of SMARCA4 mutants, SMARCA4 in the presence of SMARCA2 mutants, PARP1 in BRCA1/2 deficient cells, and/or PRMT5 in cells with a loss of MTAP, and the degrader polypeptide is selected from UBE2B, UBE2A, FBXL12, FBXL14, FBXL15, GABARAP, GABARAPL2, MAP1LC3A, KLHL6, KBTBD7, PRR20A or KLHDC2.

In some embodiments, the at least one target polypeptide is an immunology/immune-oncology target, optionally PD1/PD-L1 or JAK, and the degrader polypeptide is selected from UBE2B, UBE2A, FBXL12, FBXL14, FBXL15, GABARAP, GABARAPL2, MAP1LC3A, KLHL6, KBTBD7, PRR20A or KLHDC2.

In some embodiments, the, or the at least one, target polypeptide is dominant gain-of-function disease variant, optionally a FUS, TDP43, a-synuclein, amyloid beta precursor protein, HTT, or a prion protein gain of function disease variant, optionally selected from Table 1, and the degrader polypeptide is selected from UBE2B, UBE2A, FBXL12, FBXL14, FBXL15, GABARAP, GABARAPL2, MAP1LC3A, KLHL6, KBTBD7, PRR20A or KLHDC2.

Another aspect of the disclosure includes a process for targeted stabilization of at least one target polypeptide in at least one cell, the method comprising expressing at least one stabilizer provided in Table 4 in the, or the at least one, cell, the, or the at least one, degrader polypeptide fused to a targeting moiety. In some embodiments, the, or the at least one, stabilizer is provided in Table 5, 6 or 7. In some embodiments, the, or the at least one, stabilizer is provided in FIG. 11B. In some embodiments, the, or the at least one, stabilizer is provided in FIG. 12B.

Any sub-combination of Table 5, 6 or 7 or any other effectors described in the Figures or Examples, is contemplated.

In some embodiments, the proximity effector polypeptide is selected from GMCL1, FBXL15, PJA1, RNF115, DZIP3, RNF125, FBXO3, RNF185, RNF8, RNF183, RCHY1, KBTBD7, TRIM31, CISH, SOCS5, TRIM39, RNF144B, FBXO40, KLHL6, FBXO11, GAN, FBXL14, FBXW5, RNF111, FBXL12, BTRC, or RNF126 or selected from FBXL12, FBXL14, FBXL15, KLHDC2, KLHL6, KBTBD7, ZER1, UBE2B or KLHL40.

In some embodiments, the, or the at least one, target polypeptide is a tumor suppressor protein, optionally selected from Table 3, optionally p53 or PTEN, and the stabilizer polypeptide is selected from FBXL8, FBXO2, CDCA3, SKP1, ASB9, ELOB, KLHL40, ZFP161, KCTD17, ZBTB18, ZBTB7B, KCTD5, ZBTB20, ZBTB43, KEAP1, ZBTB10, UCHL1, OTUB1, USP39, USP38, USP14 or USP13, KLHL41, DDI1, and/or PRPS2.

In some embodiments, the, or the at least one, target polypeptide is a tumor suppressor protein, optionally selected from Table 3, optionally p53 or PTEN, and the stabilizer polypeptide is selected from KLHL40, KLHL41, DDI1, PRPS2, UCHL1, OTUB1 or USP13.

In some embodiments, the or the, or the at least one, target polypeptide is an unstable disease variant, optionally CFTR delta508, ACTB E364K, ALDOA E206K, AMHR2 R54C, AMPD3 A320V, CBS L456P, GNMT H176N, PIKLR F132L, SCARB H363N, or TPMT A80P, and the stabilizer polypeptide is selected from FBXL8, FBXO2, CDCA3, SKP1, ASB9, ELOB, KLHL40, ZFP161, KCTD17, ZBTB18, ZBTB7B, KCTD5, ZBTB20, ZBTB43, KEAP1, ZBTB10, UCHL1, OTUB1, USP39, USP38, USP14, USP13, KLHL41, DDI1, and/or PRPS2.

In some embodiments, the or the, or the at least one, target polypeptide is an unstable disease variant, optionally CFTR delta508, ACTB E364K, ALDOA E206K, AMHR2 R54C, AMPD3 A320V, CBS L456P, GNMT H176N, PIKLR F132L, SCARB H363N, or TPMT A80P, and the stabilizer polypeptide is selected from KLHL40, KLHL41, DDI1, PRPS2, UCHL1, OTUB1 or USP13.

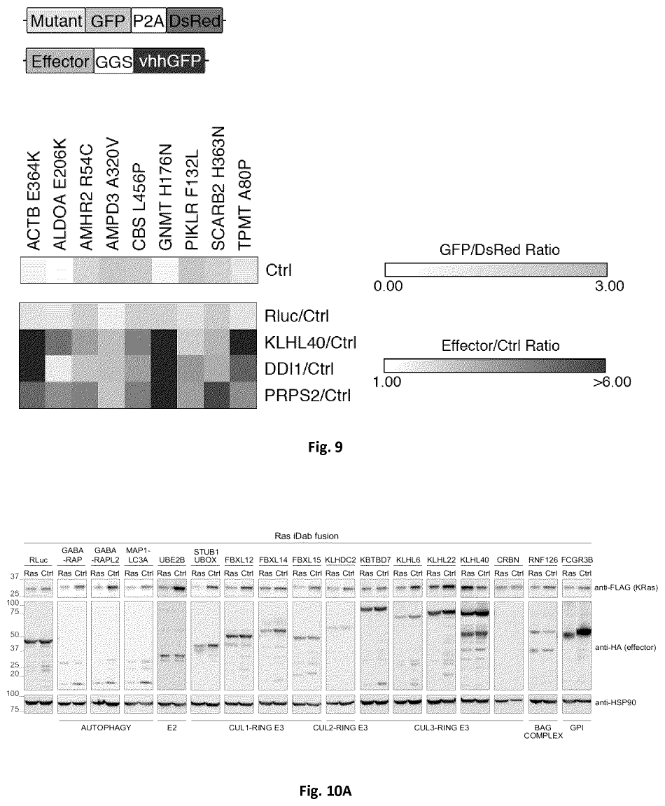

In some embodiments, the target polypeptide and effector polypeptide are those disclosed in the Examples and Figures, for example in FIG. 8. In some embodiments, the target polypeptide and effector polypeptide are used in combinations shown in the Examples and Figures, for example in FIG. 8. In some embodiments, the target polypeptides are paired/used with effector polypeptides that were shown to degrade them in the Examples and Figures, for example in FIG. 8. In other embodiments, the target polypeptides are paired with effector polypeptides that were shown to stabilize them in Examples and Figures, for example FIG. 8.

In some embodiments, the or the, or the at least one, degrader polypeptide is selected from UBE2B, UBE2A, FBXL12, FBXL14, FBXL15, GABARAP, GABARAPL2, MAP1LC3A, KLHL6, KBTBD7, and/or KLHDC2. UBE2B as a potent degrader is particularly unexpected as it is an E2 conjugating enzyme. These have not been previously used for targeted polypeptide degradation and it is often assumed that they require E3 ligases to function. The results provided in the Examples suggest that UBE2B can function without an E3 and can directly ubiquitinate its target in a proximity-dependent manner.

In some embodiments, the or the, or the at least one, degrader polypeptide is UBE2B.

In some embodiments, the or the, or the at least one, degrader polypeptide is a GPI-anchored polypeptide comprising a signal peptide, a soluble domain, and C-terminal residues 194-223 of FCGR3B (Uniprot 075015-1). In some embodiments, the or the, or the at least one, degrader polypeptide is PRNP fusion polypeptide, wherein PRNP fusion polypeptide comprises a PRNP signal peptide, a PRNP soluble domain, and C-terminal residues 194-223 of FCGR3B (Uniprot 075015-1). In some embodiments, the or the, or the at least one, degrader polypeptide is an ER resident soluble polypeptide comprising a signal peptide, a soluble domain such that it can be routed to the secretory pathway, and C-terminal residues 194-223 of FCGR3B (Uniprot 075015-1).

In some embodiments, the, or the, or the at least one, degrader polypeptide is LY6D or LYPD3. The C-terminal residues of LY6D and LYPD3 are also shown herein to be degraders and can be added to a polypeptide, optionally a GPI anchored protein, to induce degradation.

In some embodiments, the degrader polypeptide is or comprises the C-terminal residues of LY6D, e.g., residues AAPTRTALAHSALSLGLALSLLAVILAPSL (SEQ ID NO: 40).

In some embodiments, the or the, or the at least one, degrader polypeptide is a GPI-anchored polypeptide comprising a signal peptide, a soluble domain, and C-terminal residues of LY6D. In some embodiments, the degrader polypeptide comprises a GPI anchored polypeptide and AAPTRTALAHSALSLGLALSLLAVILAPSL (SEQ ID NO: 40).

In some embodiments, the degrader polypeptide is or comprises the C-terminal residues of LYPD3, e.g., residues VAPTAGLAALLLAVAAGVLL (SEQ ID NO: 41).

In some embodiments, the, or the at least one, degrader polypeptide is a GPI-anchored polypeptide comprising a signal peptide, a soluble domain, and C-terminal residues of LYPD3. In some embodiments, the degrader polypeptide comprises a GPI anchored polypeptide and VAPTAGLAALLLAVAAGVLL (SEQ ID NO: 41).

In some embodiments, the or the, or the at least one, stabilizer polypeptide is selected from of FBXL8, FBXO2, CDCA3, SKP1, ASB9, ELOB, KLHL40, ZFP161, KCTD17, ZBTB18, ZBTB7B, KCTD5, ZBTB20, ZBTB43, KEAP1, ZBTB10, KLHL41, DDI1, and/or PRPS2.

In some embodiments, the or the, or the at least one, stabilizer polypeptide is selected from of KLHL40, KLHL41, DDI1, and/or PRPS2.

In some embodiments, the or the, or the at least one, stabilizer polypeptide is KLHL40 or KLHL41. KLHL40 and KLHL41 are particularly unexpected stabilizers since they belong to a group of proteins (BTB-BACK-Kelch family) that is canonically associated with protein degradation.

In some embodiments, the or the at least one target polypeptide is an oncogene polypeptide, oncogenic fusion polypeptide, synthetic lethal target, immunology/immune-oncology target, dominant gain-of-function disease variant, tumor suppressor, and/or unstable disease variant.

In some embodiments, the oncogene/tumor suppressor polypeptide or oncogenic fusion polypeptide is RAS, MYC, or EWSR-FLI1. In some embodiments, the oncogene/tumor suppressor polypeptide or oncogenic fusion polypeptide is provided in Table 3.

In some embodiments, the synthetic lethal target is EWSR1, SMARCA2/4, PARP1, WRN, ARID1A/1B, MTAP, PKMYT1, CIP2A, APEX2, POLQ, SKP2, orATR.

In some embodiments, the immunology/immune-oncology target is PD1/PD-L1 JAK, or PTPN2.

In some embodiments, the dominant gain-of-function disease variant is FUS, TDP43, a-synuclein, amyloid beta precursor protein, HTT, or prion protein.

In some embodiments, the tumor suppressor is p53 or PTEN. In some embodiments, the tumor suppressor polypeptide is provided in Table 3.

In some embodiments, the unstable disease variant is a mutated CFTR or dystrophin variant.

In some embodiments, the targeting moiety is fused to the or the at least one stabilizer or degrader polypeptide.

In some embodiments, the targeting moiety is a nanobody, ligand or an antibody that binds the target polypeptide.

In some embodiments, the target polypeptide is a human polypeptide. In some embodiments, the or the at least one cell is a human cell.

Another aspect of the disclosure includes a method of identifying at least one putative effector polypeptide as a lethal polypeptide, the method comprising:
transducing an ORFeome library into a plurality of cells,
expressing at least one putative effector polypeptide of the ORFeome library, the or the at least one putative effector polypeptide fused to a targeting moiety that binds to the or the at least one target polypeptide, and
determining whether the or the at least one putative effector polypeptide has caused death of a cell of the transduced plurality of cells, wherein the putative effector polypeptide is a lethal polypeptide when it causes death of a cell.

In some embodiments, the determining whether the or the at least one putative effector polypeptide has caused death of cell comprises identifying where one or more putative effector polypeptides disappears from the transduced plurality of cells during the screening assay. In some embodiments, the identifying where one or more putative effector polypeptides disappears from the transduced plurality of cells during the screening assay comprises sequencing the DNA encoding the or the at least one putative effector polypeptides in cells that survived and comparing the effector genes present in the ORFeome library with the effector genes present in the cells after screening to determine which putative effector polypeptides are or are not present. In some embodiments, the identifying where one or more putative effector polypeptides disappears from the transduced plurality of cells over time comprises sequencing DNA barcodes mapped to the or the at least one putative effector polypeptides in cells that survived and comparing the effector genes present in the ORFeome library with the effector genes present in the cells after screening to determine which putative effector polypeptides are or are not present.

In some embodiments, the target polypeptide is any polypeptide may be involved in cell survival or death. In some embodiments, the target polypeptide is an oncogenic polypeptide. In some embodiments the target polypeptide is a RAS polypeptide, optionally KRAS. In some embodiments, the target polypeptide is a regulator of apoptosis. In some embodiments, the target polypeptide is a regulator of autophagy. In some embodiments, the target polypeptide is a regulator of mitophagy. In some embodiments, the target polypeptide is a regulator of other essential cellular processes which are well known in the art.

Another aspect of the disclosure includes a method of identifying at least one putative effector polypeptide as a protein trafficking polypeptide, the method comprising:
transducing an ORFeome library into a plurality of cells,
expressing at least one putative effector polypeptide of the ORFeome library, the or the at least one putative effector polypeptide to a targeting moiety that binds to the or the at least one target polypeptide, and
determining whether the or the at least one putative effector polypeptide increases cell surface localization of the or the at least one target polypeptide, wherein the putative effector polypeptide is a protein trafficking polypeptide when it increases the cell surface localization of the or the at least one target polypeptide.

In some embodiments, the target polypeptide is any cell surface polypeptide. In some embodiments, the cell surface polypeptide is a MHC class I polypeptide. In some embodiments, the target polypeptide is a mutant cell surface polypeptide. In some embodiments, the mutant cell surface polypeptide is provided in Table 2. In some embodiments, the mutant cell surface polypeptide is CFTR delta508.

TABLE 2
Examples of mutant cell surface polypeptides and their mutations

| Gene | Mutation | Gene | Mutation | Gene | Mutation | Gene | Mutation |
|---|---|---|---|---|---|---|---|
| ACVRL1 | C344P | DRD1 | T37P | LIM2 | G196E | VAPB | P56S |
| ACVRL1 | E215K | DRD1 | T37R | MC2R | C251F | VKORC1 | L128R |
| ACVRL1 | G48E | EBP | E80K | MC2R | H139Y | VKORC1 | R98W |
| ACVRL1 | L337P | EBP | G157S | MC2R | L198P | VKORC1 | V45A |
| ACVRL1 | M376R | EBP | L18P | MC2R | S74I | ZDHHC9 | R148W |
| ACVRL1 | P449L | EDN3 | A224T | MCFD2 | D81Y | CFTR | deltaF508 |
| ACVRL1 | R374W | EPHX1 | H139R | MCFD2 | Y135N | RHO | P23H |
| ACVRL1 | V380G | ERBB4 | E872K | MCOLN1 | D362Y | | |
| ADIPOQ | G15G | FBLN5 | S227P | MLC1 | A275D | | |
| ADIPOQ | R112C | FBLN5 | V60L | MLC1 | G130R | | |
| ADRB2 | A119D | FGF10 | C106F | MPDU1 | G73E | | |
| ALK | C1196M | FGF10 | G138E | MPDU1 | L119P | | |
| ALK | F1174L | FGF10 | I156R | MPDU1 | L74S | | |
| ALK | R1275Q | FGF14 | F145S | NDP | I18K | | |
| AMHR2 | H282Q | FGFR1 | V607M | NLGN3 | R451C | | |
| AMHR2 | R54C | FGFR2 | Q174P | PENK | G247D | | |
| ANG | P112L | FGFR2 | S267F | PLP1 | A247E | | |
| ANG | Q12L | FLT4 | H1035R | PLP1 | L86P | | |
| APOH | C306G | FLT4 | L1044P | PLP1 | P14L | | |
| AQP1 | N192K | FLT4 | P1114L | PLP1 | W162L | | |
| AQP1 | P38L | FSHB | C51G | PMP22 | C109R | | |
| AQP2 | G64R | FSHR | A419T | PMP22 | L16P | | |
| AQP2 | R187H | FSHR | T449I | PMP22 | L80P | | |
| AQP2 | T126M | FUCA1 | G60D | PRNP | F198V | | |
| ARL13B | R200C | FUCA1 | L405R | PTH | C18R | | |
| BSND | R8W | FUCA1 | S63L | PTH1R | H223R | | |
| BSND | V43I | G6PC | P113L | PTH1R | P132L | | |
| CAV3 | D28E | G6PC | R83C | REEP1 | P19R | | |
| CD70 | D165Y | G6PC | V338F | RGS2 | R44H | | |
| CER1 | R19W | G6PC3 | G260R | RP2 | C86Y | | |
| CFP | R319C | GABRG2 | K328M | SCARB2 | H363N | | |
| CFP | W294S | GALNS | N204K | SCNN1G | N530S | | |
| CLCN5 | G88D | GGCX | L394R | SEMA4A | F350C | | |
| CLCN5 | K725E | GGCX | W157R | SLC14A1 | N74K | | |
| CLDN19 | G20D | GNPTAB | K4Q | SLC14A1 | S291P | | |
| CLDN19 | L90P | GNRHR | Q106R | SLC15A1 | P586L | | |
| CLRN1 | C40G | GNRHR | S217R | SLC22A12 | M430T | | |
| CLRN1 | S105P | HTR1B | F124C | SLC22A12 | T217M | | |
| CNGA3 | C191Y | IL12RB1 | C186S | SLC2A1 | P485L | | |
| CNGA3 | P163L | IL12RB1 | R173P | SLC2A1 | S66F | | |
| CNGA3 | R563C | IL2RG | S108P | SLC39A4 | P200L | | |
| CNGA3 | S401P | IL2RG | W237R | SLC5A5 | G543E | | |
| COLQ | D342E | IL7R | C74Y | SLC5A5 | G93R | | |
| CPN1 | G178D | IL7R | L55Q | SLC5A5 | Q267E | | |
| CTRC | M200V | IL7R | P132S | SLC5A5 | T354P | | |
| CYP1A2 | F186L | INSR | W1227S | SLC7A9 | G105R | | |
| CYP1A2 | R456H | KCNJ10 | P194H | SLURP1 | W15R | | |
| DMD | C3207R | KEL | L597P | TACR3 | G93D | | |
| DMD | D3187G | LIM2 | F147V | TRAF3 | D463N | | |

In some embodiments, whether the or the at least one putative effector polypeptide increases cell surface localization of the or the at least one target polypeptide is determined using fluorescence-activated cell sorting (FACS) and sequencing the putative effector polypeptide identified as a protein trafficking polypeptide. In some embodiments, the FACS is performed using an antibody binding to an extracellular epitope of the or the at least one target polypeptide. In some embodiments, the or the at least one target polypeptide is fused to a FLAG tag.

Another aspect of the disclosure includes a method of treating muscular dystrophy, the method comprising administering to a subject KLHL40 or KLHL41 fused to a targeting moiety that binds a target polypeptide. KLHL40 and KLHL41 are specific to skeletal muscle, so they would be particularly useful for targeting loss-of-stability variants in e.g. muscular dystrophies. In some embodiments, the targeting moiety is a nanobody, ligand or an antibody that binds the target polypeptide, the target polypeptide being a loss of stability variant such as those in NEB, RYR1, DMD, SGCA, SGCB, SGCG, SGCD, DAG1, LAMA2, COL6A1, COL6A2, COL6A3, FLMNC, MYH7, MYH2, DES, MYOT, TTN, ACTA1, KLHL40, KLHL41, KBTBD13, TNPO3, LMNA, EMD, SYNE1, SYNE2, CAV3, BIN1, DNM2, MTM1, STIM1, STAC3, CACNA15, SPEG, CAPN3, DYSF, BIN1, ANO5, SIL1, DNAJB6, BAG3, HSPB5, TRIM32, LAMP2, VMA21, EPG5, SQSTM1, TRIM63.

In some embodiments, the target polypeptide is a human polypeptide. In some embodiments, the or the at least one cell is a human cell.

Also contemplated herein are uses of any of the methods, processes, screening assays, fusion polypeptides, cells, nucleic acids, kits or other products described herein.

TABLE 3
Examples of oncogenic polypeptides, tumor suppressor polypeptides,
and oncogenic and/or tumor suppressor fusion polypeptides

| Gene Symbol | Name | Type |
|---|---|---|
| BCR | breakpoint cluster region | fusion |
| ELN | elastin | fusion |
| EZR | ezrin | fusion |
| IGH | immunoglobulin heavy locus | fusion |
| IGK | immunoglobulin kappa locus | fusion |
| IGL | immunoglobulin lambda locus | fusion |
| IL2 | interleukin 2 | fusion |
| ITK | IL2-inducible T-cell kinase | fusion |
| MSN | moesin | fusion |
| NIN | ninein (GSK3B interacting protein) | fusion |
| OMD | osteomodulin | fusion |
| TFG | TRK-fused gene | fusion |
| TPR | translocated promoter region | fusion |
| TRA | T cell receptor alpha locus | fusion |
| TRB | T cell receptor beta locus | fusion |
| TRD | T cell receptor delta locus | fusion |
| AFF1 | AF4/FMR2 family, member 1 | fusion |
| ATIC | 5-aminoimidazole-4-carboxamide ribonucleotide | fusion |
| | formyltransferase/IMP cyclohydrolase | |
| CLP1 | cleavage and polyadenylation factor I subunit 1 | fusion |
| EML4 | echinoderm microtubule associated protein like 4 | fusion |
| ERC1 | ELKS/RAB6-interacting/CAST family member 1 | fusion |
| GAS7 | growth arrest-specific 7 | fusion |
| GMPS | guanine monphosphate synthetase | fusion |
| GOPC | golgi associated PDZ and coiled-coil motif containing | fusion |
| GPHN | gephyrin (GPH) | fusion |
| KDSR | 3-ketodihydrosphingosine reductase | fusion |
| KLK2 | kallikrein-related peptidase 2 | fusion |
| KTN1 | kinectin 1 (kinesin receptor) | fusion |
| LCP1 | lymphocyte cytosolic protein 1 (L-plastin) | fusion |
| LIFR | leukemia inhibitory factor receptor | fusion |
| LMNA | lamin A/C | fusion |
| MDS2 | myelodysplastic syndrome 2 | fusion |
| MNX1 | motor neuron and pancreas homeobox 1 | fusion |
| MUC1 | mucin 1, transmembrane | fusion |
| NACA | nascent-polypeptide-associated complex alpha polypeptide | fusion |
| NFIB | nuclear factor I/B | fusion |
| NONO | non-POU domain containing, octamer-binding | fusion |
| NSD1 | nuclear receptor binding SET domain protein 1 | fusion |
| PAX7 | paired box gene 7 | fusion |
| PAX8 | paired box gene 8 | fusion |
| PCM1 | pericentriolar material 1 (PTC4) | fusion |
| PRCC | papillary renal cell carcinoma (translocation-associated) | fusion |
| RPN1 | ribophorin I | fusion |
| SDC4 | syndecan 4 | fusion |
| SS18 | synovial sarcoma translocation, chromosome 18 | fusion |
| STRN | striatin, calmodulin binding protein | fusion |
| TFPT | TCF3 (E2A) fusion partner (in childhood leukaemia) | fusion |
| TFRC | transferrin receptor (p90, CD71) | fusion |
| TOP1 | topoisomerase (DNA) I | fusion |
| TPM4 | tropomyosin 4 | fusion |
| VAV1 | vav guanine nucleotide exchange factor 1 | fusion |
| WDCP | chromosome 2 open reading frame 44 | fusion |
| ACSL3 | acyl-CoA synthetase long-chain family member 3 | fusion |
| ACSL6 | acyl-CoA synthetase long-chain family member 6 | fusion |
| AKAP9 | A kinase (PRKA) anchor protein (yotiao) 9 | fusion |
| ALDH2 | aldehyde dehydrogenase 2 family (mitochondrial) | fusion |
| BCL7A | B-cell CLL/lymphoma 7A | fusion |
| CANT1 | calcium activated nucleotidase 1 | fusion |
| CEP89 | centrosomal protein 89 kDa | fusion |
| CHIC2 | cysteine-rich hydrophobic domain 2 | fusion |
| CLIP1 | CAP-GLY domain containing linker protein 1 | fusion |
| CNTRL | centriolin | fusion |
| COX6C | cytochrome c oxidase subunit VIc | fusion |
| CRTC3 | CREB regulated transcription coactivator 3 | fusion |
| DCTN1 | dynactin 1 | fusion |
| FNBP1 | formin binding protein 1 (FBP17) | fusion |
| HLA-A | major histocompatibility complex, class I, A | fusion |
| HOOK3 | hook homolog 3 | fusion |
| IL21R | interleukin 21 receptor | fusion |
| JAZF 1 | juxtaposed with another zinc finger gene 1 | fusion |
| KIF5B | kinesin family member 5B | fusion |
| LASP1 | LIM and SH3 protein 1 | fusion |
| MLLT1 | myeloid/lymphoid or mixed-lineage leukemia (trithorax homolog, | fusion |
| | Drosophila); translocated to, 1 (ENL) | |
| MLLT3 | myeloid/lymphoid or mixed-lineage leukemia (trithorax homolog, | fusion |
| | Drosophila); translocated to, 3 (AF9) | |
| MLLT6 | myeloid/lymphoid or mixed-lineage leukemia (trithorax homolog, | fusion |
| | Drosophila); translocated to, 6 (AF17) | |
| MYH11 | myosin, heavy polypeptide 11, smooth muscle | fusion |
| MYO5A | myosin VA (heavy chain 12, myoxin) | fusion |
| NCOA1 | nuclear receptor coactivator 1 | fusion |
| NUMA1 | nuclear mitotic apparatus protein 1 | fusion |
| PRRX1 | paired related homeobox 1 | fusion |
| RBM15 | RNA binding motif protein 15 | fusion |
| SEPTIN5 | septin 5 | fusion |
| SEPTIN6 | septin 6 | fusion |
| SEPTIN9 | septin 9 | fusion |
| SHTN1 | KIAA1598 | fusion |
| SNX29 | RUN domain containing 2A | fusion |
| TCEA1 | transcription elongation factor A (SII), 1 | fusion |
| TCF12 | transcription factor 12 (HTF4, helix-loop-helix transcription factors 4) | fusion |
| USP9X | Ubiquitin Specific Peptidase 9 X-Linked | fusion |
| VTI1A | vesicle transport through interaction with t-SNAREs homolog 1A | fusion |
| ZMYM2 | zinc finger protein 198 | fusion |
| CHCHD7 | coiled-coil-helix-coiled-coil-helix domain containing 7 | fusion |
| COL1A1 | collagen, type I, alpha 1 | fusion |
| COL2A1 | collagen, type II, alpha 1 | fusion |
| COL3A1 | collagen type III alpha 1 chain | fusion |
| DNAJB1 | DnaJ heat shock protein family (Hsp40) member B1 | fusion |
| DUX4L1 | double homeobox 4 like 1 | fusion |
| EIF4A2 | eukaryotic translation initiation factor 4A, isoform 2 | fusion |
| FIP1L1 | FIP1 like 1 (S. cerevisiae) | fusion |
| GOLGA5 | golgi autoantigen, golgin subfamily a, 5 (PTC5) | fusion |
| LHFPL6 | lipoma HMGIC fusion partner | fusion |
| LSM14A | LSM14A, SCD6 homolog A (S. cerevisiae) | fusion |
| MLLT11 | myeloid/lymphoid or mixed-lineage leukemia (trithorax homolog, | fusion |
| | Drosophila); translocated to, 11 | |
| NUP214 | nucleoporin 214 kDa (CAN) | fusion |
| NUTM2B | NUT family member 2B | fusion |
| NUTM2D | NUT family member 2A | fusion |
| PICALM | phosphatidylinositol binding clathrin assembly protein (CALM) | fusion |
| PWWP2A | PWWP domain containing 2A | fusion |
| RABEP1 | rabaptin, RAB GTPase binding effector protein 1 | fusion |
| RALGDS | ral guanine nucleotide dissociation stimulator | fusion |
| RNF213 | ring finger protein 213 | fusion |
| S100A7 | S100 calcium binding protein A7 | fusion |
| SPECC1 | sperm antigen with calponin homology and coiled-coil domains 1 | fusion |
| SRGAP3 | SLIT-ROBO Rho GTPase activating protein 3 | fusion |
| SS18L1 | synovial sarcoma translocation gene on chromosome 18-like 1 | fusion |
| THRAP3 | thyroid hormone receptor associated protein 3 (TRAP150) | fusion |
| TRIP11 | thyroid hormone receptor interactor 11 | fusion |
| ZCCHC8 | zinc finger, CCHC domain containing 8 | fusion |
| ZNF384 | zinc finger protein 384 (CIZ/NMP4) | fusion |
| ASPSCR1 | alveolar soft part sarcoma chromosome region, candidate 1 | fusion |
| FAM131B | family with sequence similarity 131, member B | fusion |
| FGFR1OP | FGFR1 oncogene partner (FOP) | fusion |
| HERPUD1 | homocysteine-inducible, endoplasmic reticulum stress-inducible, | fusion |
| | ubiquitin-like domain member 1 | |
| NCKIPSD | NCK interacting protein with SH3 domain | fusion |
| PDE4DIP | phosphodiesterase 4D interacting protein (myomegalin) | fusion |
| PPFIBP1 | PTPRF interacting protein, binding protein 1 (liprin beta 1) | fusion |
| SLC45A3 | solute carrier family 45, member 3 | fusion |
| TMPRSS2 | transmembrane protease, serine 2 | fusion |
| C15orf65 | chromosome 15 open reading frame 65 | fusion |
| HIST1H41 | histone 1, H4i (H4FM) | fusion |
| HMGN2P46 | high mobility group nucleosomal binding domain 2 pseudogene 46 | fusion |
| HSP90AA1 | heat shock protein 90 kDa alpha (cytosolic), class A member 1 | fusion |
| HSP90AB1 | heat shock protein 90 kDa alpha (cytosolic), class B member 1 | fusion |
| KIAA1549 | KIAA1549 | fusion |
| PAFAH1B2 | platelet-activating factor acetylhydrolase, isoform lb, beta subunit | fusion |
| | 30 kDa | |
| AR | Androgen Receptor | oncogene |
| JUN | jun oncogene | oncogene |
| KDR | vascular endothelial growth factor receptor 2 | oncogene |
| KIT | v-kit Hardy-Zuckerman 4 feline sarcoma viral oncogene homolog | oncogene |
| MET | met proto-oncogene (hepatocyte growth factor receptor) | oncogene |
| MPL | myeloproliferative leukaemia virus oncogene, thrombopoietin | oncogene |
| | receptor | |
| REL | v-rel reticuloendotheliosis viral oncogene homolog (avian) | oncogene |
| SKI | SKI proto-oncogene | oncogene |
| SMO | smoothened homolog (Drosophila) | oncogene |
| SRC | SRC proto-oncogene, non-receptor tyrosine kinase | oncogene |
| TNC | tenascin C | oncogene |
| WAS | Wiskott-Aldrich syndrome | oncogene |
| A1CF | APOBEC1 complementation factor | oncogene |
| AKT1 | v-akt murine thymoma viral oncogene homolog 1 | oncogene |
| AKT2 | v-akt murine thymoma viral oncogene homolog 2 | oncogene |
| AKT3 | v-akt murine thymoma viral oncogene homolog 3 | oncogene |
| ARAF | A-Raf proto-oncogene, serine/threonine kinase | oncogene |
| CALR | calreticulin | oncogene |
| CCR4 | C-C motif chemokine receptor 4 | oncogene |
| CCR7 | C-C motif chemokine receptor 7 | oncogene |
| CD28 | CD28 molecule | oncogene |
| CDK4 | cyclin-dependent kinase 4 | oncogene |
| CHD4 | chromodomain helicase DNA binding protein 4 | oncogene |
| DDR2 | discoidin domain receptor 2 | oncogene |
| EGFR | epidermal growth factor receptor (erythroblastic leukemia viral (v- | oncogene |
| | erb-b) oncogene homolog, avian) | |
| FLT3 | fms-related tyrosine kinase 3 | oncogene |
| FLT4 | fms-related tyrosine kinase 4 | oncogene |
| GNAQ | guanine nucleotide binding protein (G protein), q polypeptide | oncogene |
| GNAS | guanine nucleotide binding protein (G protein), alpha stimulating | oncogene |
| | activity polypeptide 1 | |
| GRM3 | glutamate metabotropic receptor 3 | oncogene |
| HRAS | v-Ha-ras Harvey rat sarcoma viral oncogene homolog | oncogene |
| IDH1 | isocitrate dehydrogenase 1 (NADP+), soluble | oncogene |
| DH2 | isocitrate dehydrogenase 2 (NADP+), mitochondrial | oncogene |
| IL7R | interleukin 7 receptor | oncogene |
| JAK3 | Janus kinase 3 | oncogene |
| KAT7 | lysine acetyltransferase 7 | oncogene |
| KRAS | v-Ki-ras2 Kirsten rat sarcoma 2 viral oncogene homolog | oncogene |
| MDM2 | Mdm2 p53 binding protein homolog | oncogene |
| MDM4 | Mdm4 p53 binding protein homolog | oncogene |
| MITF | melanogenesis-associated transcription factor | oncogene |
| MTOR | mechanistic target of rapamycin | oncogene |
| MUC4 | mucin 4, cell surface associated | oncogene |
| MYCL | v-myc avian myelocytomatosis viral oncogene lung carcinoma | oncogene |
| | derived homolog | |
| MYCN | v-myc myelocytomatosis viral related oncogene, neuroblastoma | oncogene |
| | derived (avian) | |
| NRAS | neuroblastoma RAS viral (v-ras) oncogene homolog | oncogene |
| RAC1 | ras-related C3 botulinum toxin substrate 1 (rho family, small GTP | oncogene |
| | binding protein Rac1) | |
| SGK1 | serum/glucocorticoid regulated kinase 1 | oncogene |
| SIX1 | SIX homeobox 1 | oncogene |
| SIX2 | SIX homeobox 2 | oncogene |
| SOX2 | SRY (sex determining region Y)-box 2 | oncogene |
| TSHR | thyroid stimulating hormone receptor | oncogene |
| UBR5 | ubiquitin protein ligase E3 component n-recognin 5 | oncogene |
| USP8 | ubiquitin specific peptidase 8 | oncogene |
| XPO1 | exportin 1 (CRM1 homolog, yeast) | oncogene |
| ZEB1 | zinc finger E-box binding homeobox 1 | oncogene |
| ACVR1 | activin A receptor, type I | oncogene |
| CCNE1 | cyclin E1 | oncogene |
| CD79A | CD79a molecule, immunoglobulin-associated alpha | oncogene |
| CD79B | CD79b molecule, immunoglobulin-associated beta | oncogene |
| CDH17 | cadherin 17 | oncogene |
| CSF1R | colony stimulating factor 1 receptor | oncogene |
| CSF3R | colony stimulating factor 3 receptor (granulocyte) | oncogene |
| CXCR4 | C-X-C motif chemokine receptor 4 | oncogene |
| DGCR8 | DGCR8, microprocessor complex subunit | oncogene |
| ERBB3 | erb-b2 receptor tyrosine kinase 3 | oncogene |
| FGFR4 | fibroblast growth factor receptor 4 | oncogene |
| FOXA1 | forkhead box A1 | oncogene |
| FUBP1 | far upstream element (FUSE) binding protein 1 | oncogene |
| GATA2 | GATA binding protein 2 | oncogene |
| GNA11 | guanine nucleotide binding protein (G protein), alpha 11 (Gq class) | oncogene |
| H3F3A | H3 histone, family 3A | oncogene |
| H3F3B | H3 histone, family 3B (H3.3B) | oncogene |
| HIF1A | hypoxia inducible factor 1 alpha subunit | oncogene |
| IKBKB | inhibitor of kappa light polypeptide gene enhancer in B-cells, kinase | oncogene |
| | beta | |
| IL6ST | interleukin 6 signal transducer (gp130, oncostatin M receptor) | oncogene |
| KCNJ5 | potassium inwardly-rectifying channel; subfamily J; member 5 | oncogene |
| MACC1 | MET transcriptional regulator MACC1 | oncogene |
| MAPK1 | mitogen-activated protein kinase 1 | oncogene |
| MUC16 | mucin 16, cell surface associated | oncogene |
| MYD88 | myeloid differentiation primary response gene (88) | oncogene |
| MYOD1 | myogenic differentiation 1 | oncogene |
| NT5C2 | 5′-nucleotidase, cytosolic II | oncogene |
| PPM1D | protein phosphatase, Mg2+/Mn2+ dependent 1D | oncogene |
| PREX2 | phosphatidylinositol-3,4,5-trisphosphate dependent Rac exchange | oncogene |
| | factor 2 | |
| SALL4 | spalt like transcription factor 4 | oncogene |
| SF3B1 | splicing factor 3b, subunit 1, 155 kDa | oncogene |
| SRSF2 | serine/arginine-rich splicing factor 2 | oncogene |
| STAT3 | signal transducer and activator of transcription 3 (acute-phase | oncogene |
| | response factor) | |
| TRRAP | transformation/transcription domain-associated protein | oncogene |
| U2AF1 | U2 small nuclear RNA auxiliary factor 1 | oncogene |
| CARD11 | caspase recruitment domain family, member 11 | oncogene |
| CTNNA2 | catenin alpha 2 | oncogene |
| CTNND2 | catenin delta 2 | oncogene |
| KNSTRN | kinetochore localized astrin/SPAG5 binding protein | oncogene |
| MAP2K1 | mitogen-activated protein kinase kinase 1 | oncogene |
| MAP2K2 | mitogen-activated protein kinase kinase 2 | oncogene |
| PIK3CA | phosphoinositide-3-kinase, catalytic, alpha polypeptide | oncogene |
| PIK3CB | phosphatidylinositol-4,5-bisphosphate 3-kinase catalytic subunit | oncogene |
| | beta | |
| PRKACA | protein kinase cAMP-activated catalytic subunit alpha | oncogene |
| PTPN11 | protein tyrosine phosphatase, non-receptor type 11 | oncogene |
| SETDB1 | SET domain bifurcated 1 | oncogene |
| TMSB4X | Thymosin Beta 4 X-Linked | oncogene |
| ARHGAP5 | Rho GTPase activating protein 5 | oncogene |
| BCL2L12 | BCL2 like 12 | oncogene |
| CACNA1D | calcium channel, voltage-dependent, L type, alpha 1D subunit | oncogene |
| CYSLTR2 | cysteinyl leukotriene receptor 2 | oncogene |
| HIST1H3B | histone cluster 1, H3b | oncogene |
| ALK | anaplastic lymphoma kinase (Ki-1) | oncogene, fusion |
| DEK | DEK oncogene (DNA binding) | oncogene, fusion |
| ERG | v-ets erythroblastosis virus E26 oncogene like (avian) | oncogene, fusion |
| FEV | FEV protein - (HSRNAFEV) | oncogene, fusion |
| HLF | hepatic leukemia factor | oncogene, fusion |
| LCK | lymphocyte-specific protein tyrosine kinase | oncogene, fusion |
| LPP | LIM domain containing preferred translocation partner in lipoma | oncogene, fusion |
| LYN | LYN Proto-Oncogene | oncogene, fusion |
| MAF | v-maf musculoaponeurotic fibrosarcoma oncogene homolog | oncogene, fusion |
| MN1 | MN1 proto-oncogene, transcriptional regulator | oncogene, fusion |
| MYB | v-myb myeloblastosis viral oncogene homolog | oncogene, fusion |
| MYC | v-myc myelocytomatosis viral oncogene homolog (avian) | oncogene, fusion |
| RET | ret proto-oncogene | oncogene, fusion |
| SET | SET translocation | oncogene, fusion |
| SYK | spleen tyrosine kinase | oncogene, fusion |
| TEC | tec protein tyrosine kinase | oncogene, fusion |
| ABL1 | v-abl Abelson murine leukemia viral oncogene homolog 1 | oncogene, fusion |
| ABL2 | c-abl oncogene 2, non-receptor tyrosine kinase | oncogene, fusion |
| AFDN | myeloid/lymphoid or mixed-lineage leukemia (trithorax homolog, | oncogene, fusion |
| | Drosophila); translocated to, 4 (AF6) | |
| AFF3 | AF4/FMR2 family, member 3 | oncogene, fusion |
| AFF4 | AF4/FMR2 family, member 4 | oncogene, fusion |
| ATF1 | activating transcription factor 1 | oncogene, fusion |
| BCL2 | B-cell CLL/lymphoma 2 | oncogene, fusion |
| BCL3 | B-cell CLL/lymphoma 3 | oncogene, fusion |
| BCL6 | B-cell CLL/lymphoma 6 | oncogene, fusion |
| BCL9 | B-cell CLL/lymphoma 9 | oncogene, fusion |
| BRAF | v-raf murine sarcoma viral oncogene homolog B1 | oncogene, fusion |
| BRD3 | bromodomain containing 3 | oncogene, fusion |
| BRD4 | bromodomain containing 4 | oncogene, fusion |
| CD74 | CD74 molecule, major histocompatibility complex, class II invariant | oncogene, fusion |
| | chain | |
| CDK6 | cyclin-dependent kinase 6 | oncogene, fusion |
| DDX5 | DEAD (Asp-Glu-Ala-Asp) box polypeptide 5 | oncogene, fusion |
| DDX6 | DEAD (Asp-Glu-Ala-Asp) box polypeptide 6 | oncogene, fusion |
| ELK4 | ELK4, ETS-domain protein (SRF accessory protein 1) | oncogene, fusion |
| ETV1 | ets variant gene 1 | oncogene, fusion |
| ETV4 | ets variant gene 4 (E1A enhancer binding protein, E1AF) | oncogene, fusion |
| ETV5 | ets variant gene 5 | oncogene, fusion |
| FLI1 | Friend leukemia virus integration 1 | oncogene, fusion |
| GLI1 | GLI family zinc finger 1 | oncogene, fusion |
| HEY1 | hairy/enhancer-of-split related with YRPW motif 1 | oncogene, fusion |
| HIP1 | huntingtin interacting protein 1 | oncogene, fusion |
| JAK2 | Janus kinase 2 | oncogene, fusion |
| LMO1 | LIM domain only 1 (rhombotin 1) (RBTN1) | oncogene, fusion |
| LMO2 | LIM domain only 2 (rhombotin-like 1) (RBTN2) | oncogene, fusion |
| LYL1 | lymphoblastic leukemia derived sequence 1 | oncogene, fusion |
| MAFB | v-maf musculoaponeurotic fibrosarcoma oncogene homolog B | oncogene, fusion |
| | (avian) | |
| MSI2 | musashi homolog 2 (Drosophila) | oncogene, fusion |
| NPM1 | nucleophosmin (nucleolar phosphoprotein B23, numatrin) | oncogene, fusion |
| NSD2 | Wolf-Hirschhorn syndrome candidate 1(MMSET) | oncogene, fusion |
| NSD3 | Wolf-Hirschhorn syndrome candidate 1-like 1 (NSD3) | oncogene, fusion |
| PAX3 | paired box gene 3 | oncogene, fusion |
| PBX1 | pre-B-cell leukemia transcription factor 1 | oncogene, fusion |
| PIM1 | pim-1 oncogene | oncogene, fusion |
| RAF1 | v-raf-1 murine leukemia viral oncogene homolog 1 | oncogene, fusion |
| RARA | retinoic acid receptor, alpha | oncogene, fusion |
| ROS1 | v-ros UR2 sarcoma virus oncogene homolog 1 (avian) | oncogene, fusion |
| SND1 | staphylococcal nuclease and tudor domain containing 1 | oncogene, fusion |
| SSX1 | synovial sarcoma, X breakpoint 1 | oncogene, fusion |
| SSX2 | synovial sarcoma, X breakpoint 2 | oncogene, fusion |
| SSX4 | synovial sarcoma, X breakpoint 4 | oncogene, fusion |
| STIL | SCL/TAL1 interrupting locus | oncogene, fusion |
| TAL1 | T-cell acute lymphocytic leukemia 1 (SCL) | oncogene, fusion |
| TAL2 | T-cell acute lymphocytic leukemia 2 | oncogene, fusion |
| TFE3 | transcription factor binding to IGHM enhancer 3 | oncogene, fusion |
| TFEB | transcription factor EB | oncogene, fusion |
| TLX1 | T-cell leukemia, homeobox 1 (HOX11) | oncogene, fusion |
| TLX3 | T-cell leukemia, homeobox 3 (HOX11L2) | oncogene, fusion |
| USP6 | ubiquitin specific peptidase 6 (Tre-2 oncogene) | oncogene, fusion |
| ACKR3 | atypical chemokine receptor 3 | oncogene, fusion |
| BIRC6 | baculoviral IAP repeat containing 6 | oncogene, fusion |
| CCND1 | cyclin D1 | oncogene, fusion |
| CCND2 | cyclin D2 | oncogene, fusion |
| CCND3 | cyclin D3 | oncogene, fusion |
| CREB1 | cAMP responsive element binding protein 1 | oncogene, fusion |
| CRLF2 | cytokine receptor-like factor 2 | oncogene, fusion |
| CRTC1 | CREB regulated transcription coactivator 1 | oncogene, fusion |
| DDIT3 | DNA-damage-inducible transcript 3 | oncogene, fusion |
| ERBB2 | v-erb-b2 erythroblastic leukemia viral oncogene homolog 2, | oncogene, fusion |
| | neuro/glioblastoma derived oncogene homolog (avian) | |
| EWSR1 | Ewing sarcoma breakpoint region 1 (EWS) | oncogene, fusion |
| FCRL4 | Fc receptor-like 4 | oncogene, fusion |
| FGFR1 | fibroblast growth factor receptor 1 | oncogene, fusion |
| FGFR2 | fibroblast growth factor receptor 2 | oncogene, fusion |
| FGFR3 | fibroblast growth factor receptor 3 | oncogene, fusion |
| FOXP1 | forkhead box P1 | oncogene, fusion |
| FOXR1 | forkhead box R1 | oncogene, fusion |
| FSTL3 | follistatin-like 3 (secreted glycoprotein) | oncogene, fusion |
| HMGA1 | high mobility group AT-hook 1 | oncogene, fusion |
| HMGA2 | high mobility group AT-hook 2 (HMGIC) | oncogene, fusion |
| KAT6A | K(lysine) acetyltransferase 6A | oncogene, fusion |
| KDM5A | lysine (K)-specific demethylase 5A, JARID1A | oncogene, fusion |
| KMT2A | lysine (K)-specific methyltransferase 2A | oncogene, fusion |
| MALT1 | mucosa associated lymphoid tissue lymphoma translocation gene 1 | oncogene, fusion |
| MAML2 | mastermind-like 2 (Drosophila) | oncogene, fusion |
| MECOM | MDS1 and EVI1 complex locus | oncogene, fusion |
| MTCP1 | mature T-cell proliferation 1 | oncogene, fusion |
| NCOA2 | nuclear receptor coactivator 2 (TIF2) | oncogene, fusion |
| NR4A3 | nuclear receptor subfamily 4, group A, member 3 (NOR1) | oncogene, fusion |
| NTRK3 | neurotrophic tyrosine kinase, receptor, type 3 | oncogene, fusion |
| NUP98 | nucleoporin 98 kDa | oncogene, fusion |
| NUTM1 | NUT midline carcinoma, family member 1 | oncogene, fusion |
| OLIG2 | oligodendrocyte lineage transcription factor 2 (BHLHB1) | oncogene, fusion |
| P2RY8 | purinergic receptor P2Y, G-protein coupled, 8 | oncogene, fusion |
| PDGFB | platelet-derived growth factor beta polypeptide (simian sarcoma | oncogene, fusion |
| | viral (v-sis) oncogene homolog) | |
| PLAG1 | pleiomorphic adenoma gene 1 | oncogene, fusion |
| PLCG1 | phospholipase C, gamma 1 | oncogene, fusion |
| PSIP1 | PC4 and SFRS1 interacting protein 1 (LEDGF) | oncogene, fusion |
| RSPO3 | R-spondin 3 | oncogene, fusion |
| SRSF3 | serine/arginine-rich splicing factor 3 | oncogene, fusion |
| STAT6 | signal transducer and activator of transcription 6, interleukin-4 | oncogene, fusion |
| | induced | |
| TAF15 | TAF15 RNA polymerase II, TATA box binding protein (TBP)- | oncogene, fusion |
| | associated factor, 68 kDa | |
| TCL1A | T-cell leukemia/lymphoma 1A | oncogene, fusion |
| WWTR1 | WW domain containing transcription regulator 1 | oncogene, fusion |
| BCL11A | B-cell CLL/lymphoma 11A | oncogene, fusion |
| CHST11 | carbohydrate sulfotransferase 11 | oncogene, fusion |
| CTNNB1 | catenin (cadherin-associated protein), beta 1 | oncogene, fusion |
| FCGR2B | Fc fragment of IgG, low affinity IIb, receptor for (CD32) | oncogene, fusion |
| HOXA13 | homeo box A13 | oncogene, fusion |
| HOXC11 | homeo box C11 | oncogene, fusion |
| HOXC13 | homeo box C13 | oncogene, fusion |
| HOXD11 | homeo box D11 | oncogene, fusion |
| HOXD13 | homeo box D13 | oncogene, fusion |
| MLLT10 | myeloid/lymphoid or mixed-lineage leukemia (trithorax homolog, | oncogene, fusion |
| | Drosophila); translocated to, 10 (AF10) | |
| NFATC2 | nuclear factor of activated T-cells, cytoplasmic, calcineurin- | oncogene, fusion |
| | dependent 2 | |
| PDGFRA | platelet-derived growth factor, alpha-receptor | oncogene, fusion |
| PDGFRB | platelet-derived growth factor receptor, beta polypeptide | oncogene, fusion |
| POU5F1 | POU domain, class 5, transcription factor 1 | oncogene, fusion |
| PRDM16 | PR domain containing 16 | oncogene, fusion |
| SETBP1 | SET binding protein 1 | oncogene, fusion |
| SH3GL1 | SH3-domain GRB2-like 1 (EEN) | oncogene, fusion |
| TCF7L2 | transcription factor 7-like 2 | oncogene, fusion |
| TRIM27 | tripartite motif-containing 27 | oncogene, fusion |
| ZNF521 | zinc finger protein 521 | oncogene, fusion |
| CREB3L2 | cAMP responsive element binding protein 3-like 2 | oncogene, fusion |
| POU2AF1 | POU domain, class 2, associating factor 1 (OBF1) | oncogene, fusion |
| PDCD1LG2 | programmed cell death 1 ligand 2 | oncogene, fusion |
| RAP1GDS1 | RAP1, GTP-GDP dissociation stimulator 1 | oncogene, fusion |
| TNFRSF17 | tumor necrosis factor receptor superfamily, member 17 | oncogene, fusion |
| HNRNPA2B1 | heterogeneous nuclear ribonucleoprotein A2/B1 | oncogene, fusion |
| BTK | Bruton agammaglobulinemia tyrosine kinase | oncogene, TSG (tumor |
| | | suppressor gene) |
| FES | FES proto-oncogene, tyrosine kinase | oncogene, TSG |
| QKI | QKI, KH domain containing, RNA binding | oncogene, TSG |
| CBLC | Cas-Br-M (murine) ecotropic retroviral transforming sequence c | oncogene, TSG |
| CUX1 | cut-like homeobox 1 | oncogene, TSG |
| DAXX | death-domain associated protein | oncogene, TSG |
| DDB2 | damage-specific DNA binding protein 2 | oncogene, TSG |
| EZH2 | enhancer of zeste homolog 2 | oncogene, TSG |
| GPC3 | glypican 3 | oncogene, TSG |
| IRS4 | insulin receptor substrate 4 | oncogene, TSG |
| JAK1 | Janus kinase 1 | oncogene, TSG |
| KLF4 | Kruppel-like factor 4 | oncogene, TSG |
| LEF1 | lymphoid enhancer binding factor 1 | oncogene, TSG |
| POLQ | DNA polymerase theta | oncogene, TSG |
| PTK6 | protein tyrosine kinase 6 | oncogene, TSG |
| RHOA | ras homolog family member A | oncogene, TSG |
| TBX3 | T-box 3 | oncogene, TSG |
| TERT | telomerase reverse transcriptase | oncogene, TSG |
| TP63 | tumor protein p63 | oncogene, TSG |
| BCL9L | B-cell CLL/lymphoma 9-like | oncogene, TSG |
| EPAS1 | endothelial PAS domain protein 1 | oncogene, TSG |
| ERBB4 | erb-b2 receptor tyrosine kinase 4 | oncogene, TSG |
| FOXL2 | forkhead box L2 | oncogene, TSG |
| GATA1 | GATA binding protein 1 (globin transcription factor 1) | oncogene, TSG |
| GATA3 | GATA binding protein 3 | oncogene, TSG |
| IKZF3 | IKAROS Family Zinc Finger 3 | oncogene, TSG |
| KDM6A | lysine (K)-specific demethylase 6A, UTX | oncogene, TSG |
| KMT2D | lysine (K)-specific methyltransferase 2D | oncogene, TSG |
| RAD21 | RAD21 homolog (S. pombe) | oncogene, TSG |
| ATP1A1 | ATPase, Na+/K+ transporting, alpha 1 polypeptide | oncogene, TSG |
| BCORL1 | BCL6 corepressor-like 1 | oncogene, TSG |
| BMPR1A | bone morphogenetic protein receptor, type IA | oncogene, TSG |
| CDKN1A | cyclin dependent kinase inhibitor 1A | oncogene, TSG |
| MAP2K4 | mitogen-activated protein kinase kinase 4 | oncogene, TSG |
| MAP3K1 | mitogen-activated protein kinase kinase kinase 1, E3 ubiquitin | oncogene, TSG |
| | protein ligase | |
| NFE2L2 | nuclear factor (erythroid-derived 2)-like 2 (NRF2) | oncogene, TSG |
| NKX2-1 | NK2 homeobox 1 | oncogene, TSG |
| NOTCH2 | Notch homolog 2 | oncogene, TSG |
| PABPC1 | poly(A) binding protein cytoplasmic 1 | oncogene, TSG |
| RECQL4 | RecQ protein-like 4 | oncogene, TSG |
| MAP3K13 | mitogen-activated protein kinase kinase kinase 13 | oncogene, TSG |
| APOBEC3B | apolipoprotein B mRNA editing enzyme catalytic subunit 3B | oncogene, TSG |
| CBL | Cas-Br-M (murine) ecotropic retroviral transforming | oncogene, TSG, fusion |
| CIC | capicua homolog | oncogene, TSG, fusion |
| WT1 | Wilms tumour 1 gene | oncogene, TSG, fusion |
| ARNT | aryl hydrocarbon receptor nuclear translocator | oncogene, TSG, fusion |
| ELF4 | E74-like factor 4 (ets domain transcription factor) | oncogene, TSG, fusion |
| ESR1 | estrogen receptor 1 | oncogene, TSG, fusion |
| IRF4 | interferon regulatory factor 4 | oncogene, TSG, fusion |
| PAX5 | paired box gene 5 (B-cell lineage specific activator protein) | oncogene, TSG, fusion |
| TCF3 | transcription factor 3 (E2A immunoglobulin enhancer binding | oncogene, TSG, fusion |
| | factors E12/E47) | |
| TET1 | tet methylcytosine dioxygenase 1 | oncogene, TSG, fusion |
| TP53 | tumor protein p53 | oncogene, TSG, fusion |
| BIRC3 | baculoviral IAP repeat-containing 3 | oncogene, TSG, fusion |
| FOXO1 | forkhead box O1 | oncogene, TSG, fusion |
| FOXO3 | forkhead box O3 | oncogene, TSG, fusion |
| FOXO4 | forkhead box O4 | oncogene, TSG, fusion |
| HOXA9 | homeo box A9 | oncogene, TSG, fusion |
| MRTFA | megakaryoblastic leukemia (translocation) 1 | oncogene, TSG, fusion |
| NFKB2 | nuclear factor of kappa light polypeptide gene enhancer in B-cells 2 | oncogene, TSG, fusion |
| | (p49/p100) | |
| NTRK1 | neurotrophic tyrosine kinase, receptor, type 1 | oncogene, TSG, fusion |
| RUNX1 | runt-related transcription factor 1 (AML1) | oncogene, TSG, fusion |
| SUZ12 | suppressor of zeste 12 homolog (Drosophila) | oncogene, TSG, fusion |
| BCL11B | B-cell CLL/lymphoma 11B (CTIP2) | oncogene, TSG, fusion |
| CREBBP | CREB binding protein (CBP) | oncogene, TSG, fusion |
| HOXA11 | homeo box A11 | oncogene, TSG, fusion |
| MALAT1 | metastasis associated lung adenocarcinoma transcript 1 ( Inc-RNA; | oncogene, TSG, fusion |
| | non-protein coding) | |
| NOTCH1 | Notch homolog 1, translocation-associated (Drosophila) (TAN1) | oncogene, TSG, fusion |
| STAT5B | signal transducer and activator of transcription 5B | oncogene, TSG, fusion |
| TRIM24 | tripartite motif containing 24 | oncogene, TSG, fusion |
| PRKAR1A | protein kinase, cAMP-dependent, regulatory, type I, alpha (tissue | oncogene, TSG, fusion |
| | specific extinguisher 1) | |
| RUNX1T1 | runt-related transcription factor 1; translocated to, 1 (cyclin D- | oncogene, TSG, fusion |
| | related) | |
| TBL1XR1 | transducin (beta)-like 1 X-linked receptor 1 | oncogene, TSG, fusion |
| FH | fumarate hydratase | TSG |
| APC | adenomatous polyposis of the colon gene | TSG |
| ATM | ataxia telangiectasia mutated | TSG |
| ATR | ATR serine/threonine kinase | TSG |
| B2M | beta-2-microglobulin | TSG |
| BAX | BCL2 associated X, apoptosis regulator | TSG |
| BLM | Bloom Syndrome | TSG |
| EED | embryonic ectoderm development | TSG |
| FAS | Fas cell surface death receptor | TSG |
| ID3 | inhibitor of DNA binding 3, HLH protein | TSG |
| MAX | Myc associated factor X | TSG |
| NBN | nibrin | TSG |
| NF2 | neurofibromatosis type 2 gene | TSG |
| RB1 | retinoblastoma gene | TSG |
| VHL | von Hippel-Lindau syndrome gene | TSG |
| WRN | Werner syndrome (RECQL2) | TSG |
| XPA | xeroderma pigmentosum, complementation group A | TSG |
| XPC | xeroderma pigmentosum, complementation group C | TSG |
| ATRX | alpha thalassemia/mental retardation syndrome X-linked | TSG |
| BAP1 | BRCA1 associated protein-1 (ubiquitin carboxy-terminal hydrolase) | TSG |
| BTG2 | BTG Anti-Proliferation Factor 2 | TSG |
| CBLB | Cas-Br-M (murine) ecotropic retroviral transforming sequence b | TSG |
| CCNC | cyclin C | TSG |
| CDH1 | cadherin 1, type 1, E-cadherin (epithelial) (ECAD) | TSG |
| CHD2 | chromodomain helicase DNA binding protein 2 | TSG |
| CTCF | CCCTC-binding factor | TSG |
| CUL3 | cullin 3 | TSG |
| CYLD | familial cylindromatosis gene | TSG |
| DNM2 | dynamin 2 | TSG |
| ELF3 | E74 like ETS transcription factor 3 | TSG |
| EXT2 | multiple exostoses type 2 gene | TSG |
| FAT1 | FAT atypical cadherin 1 | TSG |
| FAT4 | FAT atypical cadherin 4 | TSG |
| FEN1 | flap structure-specific endonuclease 1 | TSG |
| FLCN | folliculin | TSG |
| GPC5 | glypican 5 | TSG |
| KLF6 | Kruppel-like factor 6 | TSG |
| MEN1 | multiple endocrine neoplasia type 1 gene | TSG |
| MGMT | O-6-methylguanine-DNA methyltransferase | TSG |
| MLH1 | E. coli MutL homolog gene | TSG |
| MSH2 | mutS homolog 2 (E. coli) | TSG |
| MSH6 | mutS homolog 6 (E. coli) | TSG |
| PHF6 | PHD finger protein 6 | TSG |
| PMS2 | PMS2 postmeiotic segregation increased 2 (S. cerevisiae) | TSG |
| POLE | polymerase (DNA directed), epsilon, catalytic subunit | TSG |
| POLG | DNA polymerase gamma, catalytic subunit | TSG |
| POT1 | protection of telomeres 1 | TSG |
| PRF1 | perforin 1 (pore forming protein) | TSG |
| PTEN | phosphatase and tensin homolog gene | TSG |
| RPL5 | ribososomal protein L5 | TSG |
| SBDS | Shwachman-Bodian-Diamond syndrome protein | TSG |
| SDHA | succinate dehydrogenase complex, subunit A, flavoprotein (Fp) | TSG |
| SDHB | succinate dehydrogenase complex, subunit B, iron sulfur (Ip) | TSG |
| SDHC | succinate dehydrogenase complex, subunit C, integral membrane | TSG |
| | protein, 15 kDa | |
| SDHD | succinate dehydrogenase complex, subunit D, integral membrane | TSG |
| | protein | |
| SPEN | spen family transcriptional repressor | TSG |
| SPOP | speckle type POZ protein | TSG |
| SUFU | suppressor of fused homolog (Drosophila) | TSG |
| TET2 | tet oncogene family member 2 | TSG |
| TSC1 | tuberous sclerosis 1 gene | TSG |
| TSC2 | tuberous sclerosis 2 gene | TSG |
| WNK2 | WNK lysine deficient protein kinase 2 | TSG |
| AMER1 | APC membrane recruitment protein 1 | TSG |
| ARID2 | AT rich interactive domain 2 | TSG |
| ASXL1 | additional sex combs like 1 | TSG |
| ASXL2 | additional sex combs like 2, transcriptional regulator | TSG |
| AXIN1 | axin 1 | TSG |
| AXIN2 | axin 2 | TSG |
| BARD1 | BRCA1 associated RING domain 1 | TSG |
| BAZ1A | bromodomain adjacent to zinc finger domain 1A | TSG |
| BRCA1 | familial breast/ovarian cancer gene 1 | TSG |
| BRCA2 | familial breast/ovarian cancer gene 2 | TSG |
| BRIP1 | BRCA1 interacting protein C-terminal helicase 1 | TSG |
| BUB1B | BUB1 budding uninhibited by benzimidazoles 1 homolog beta | TSG |
| | (yeast) | |
| CASP3 | caspase 3 | TSG |
| CASP8 | caspase 8, apoptosis-related cysteine peptidase | TSG |
| CASP9 | caspase 9 | TSG |
| CDC73 | cell division cycle 73 | TSG |
| CDH10 | cadherin 10 | TSG |
| CDK12 | cyclin-dependent kinase 12 | TSG |
| CEBPA | CCAAT/enhancer binding protein (C/EBP), alpha | TSG |
| CHEK2 | CHK2 checkpoint homolog (S. pombe) | TSG |
| CNOT3 | CCR4-NOT transcription complex subunit 3 | TSG |
| CPEB3 | cytoplasmic polyadenylation element binding protein 3 | TSG |
| CSMD3 | CUB and Sushi multiple domains 3 | TSG |
| DDX3X | DEAD-box helicase 3, X-linked | TSG |
| ERCC2 | excision repair cross-complementing rodent repair deficiency, | TSG |
| | complementation group 2 (xeroderma pigmentosum D) | |
| ERCC3 | excision repair cross-complementing rodent repair deficiency, | TSG |
| | complementation group 3 (xeroderma pigmentosum group B | |
| | complementing) | |
| ERCC4 | excision repair cross-complementing rodent repair deficiency, | TSG |
| | complementation group 4 | |
| ERCC5 | excision repair cross-complementing rodent repair deficiency, | TSG |
| | complementation group 5 (xeroderma pigmentosum, | |
| | complementation group G (Cockayne syndrome)) | |
| ETNK1 | ethanolamine kinase 1 | TSG |
| FANCA | Fanconi anemia, complementation group A | TSG |
| FANCC | Fanconi anemia, complementation group C | TSG |
| FANCE | Fanconi anemia, complementation group E | TSG |
| FANCF | Fanconi anemia, complementation group F | TSG |
| FANCG | Fanconi anemia, complementation group G | TSG |
| FBLN2 | fibulin 2 | TSG |
| FBXW7 | F-box and WD-40 domain protein 7 (archipelago homolog, | TSG |
| | Drosophila) | |
| HNF1A | HNF1 homeobox A | TSG |
| KDM5C | lysine (K)-specific demethylase 5C (JARID1C) | TSG |
| KEAP1 | kelch like ECH associated protein 1 | TSG |
| KMT2C | lysine (K)-specific methyltransferase 2C | TSG |
| LATS1 | large tumor suppressor kinase 1 | TSG |
| LATS2 | large tumor suppressor kinase 2 | TSG |
| LRP1B | LDL receptor related protein 1B | TSG |
| LZTR1 | leucine-zipper-like transcription regulator 1 | TSG |
| MED12 | mediator complex subunit 12 | TSG |
| MUTYH | mutY homolog (E. coli) | TSG |
| N4BP2 | NEDD4 binding protein 2 | TSG |
| NCOR1 | nuclear receptor corepressor 1 | TSG |
| NCOR2 | nuclear receptor corepressor 2 | TSG |
| NTHL1 | nth like DNA glycosylase 1 | TSG |
| PALB2 | partner and localizer of BRCA2 | TSG |
| PBRM1 | polybromo 1 | TSG |
| POLD1 | DNA polymerase delta 1, catalytic subunit | TSG |
| PPP6C | protein phosphatase 6, catalytic subunit | TSG |
| PRDM1 | PR domain containing 1, with ZNF domain | TSG |
| PRDM2 | PR/SET domain 2 | TSG |
| PTCH1 | Homolog of Drosophila Patched gene | TSG |
| PTPN6 | protein tyrosine phosphatase, non-receptor type 6 | TSG |
| PTPRB | protein tyrosine phosphatase, receptor type, B | TSG |
| PTPRC | protein tyrosine phosphatase, receptor type, C | TSG |
| PTPRD | protein tyrosine phosphatase, receptor type D | TSG |
| PTPRT | protein tyrosine phosphatase, receptor type T | TSG |
| RAD17 | RAD17 checkpoint clamp loader component | TSG |
| RBM10 | RNA binding motif protein 10 | TSG |
| RFWD3 | ring finger and WD repeat domain 3 | TSG |
| RNF43 | ring finger protein 43 | TSG |
| ROBO2 | roundabout guidance receptor 2 | TSG |
| RPL10 | ribosomal protein L10 | TSG |
| SETD2 | SET domain containing 2 | TSG |
| SFRP4 | secreted frizzled related protein 4 | TSG |
| SH2B3 | SH2B adaptor protein 3 | TSG |
| SIRPA | signal regulatory protein alpha | TSG |
| SMAD2 | SMAD family member 2 | TSG |
| SMAD3 | SMAD family member 3 | TSG |
| SMAD4 | SMAD family member 4 | TSG |
| SMC1A | structural maintenance of chromosomes 1A | TSG |
| SOCS1 | suppressor of cytokine signaling 1 | TSG |
| SOX21 | SRY-box 21 | TSG |
| STAG1 | stromal antigen 1 | TSG |
| STAG2 | stromal antigen 2 | TSG |
| STK11 | serine/threonine kinase 11 gene (LKB1) | TSG |
| TRAF7 | tumour necrosis factor receptor-associated factor 7 | TSG |
| USP44 | ubiquitin specific peptidase 44 | TSG |
| ZFHX3 | zinc finger homeobox 3 | TSG |
| ZMYM3 | zinc finger MYM-type containing 3 | TSG |
| ZNRF3 | zinc and ring finger 3 | TSG |
| ZRSR2 | zinc finger (CCCH type), RNA-binding motif and serine/arginine rich 2 | TSG |
| ACVR2A | activin A receptor type 2A | TSG |
| ARID1B | AT rich interactive domain 1B | TSG |
| ATP2B3 | ATPase, Ca++ transporting, plasma membrane 3 | TSG |
| CDKN1B | cyclin-dependent kinase inhibitor 1B (p27, Kip1) | TSG |
| CDKN2A | cyclin-dependent kinase inhibitor 2A (p16(INK4a)) gene | TSG |
| CDKN2C | cyclin-dependent kinase inhibitor 2C (p18, inhibits CDK4) | TSG |
| DICER1 | dicer 1, ribonuclease type III | TSG |
| DNMT3A | DNA (cytosine-5-)-methyltransferase 3 alpha | TSG |
| DROSHA | drosha ribonuclease III | TSG |
| FANCD2 | Fanconi anemia, complementation group D2 | TSG |
| FBXO11 | F-box protein 11 | TSG |
| GRIN2A | glutamate receptor, ionotropic, N-methyl D-aspartate 2A | TSG |
| LARP4B | La ribonucleoprotein domain family member 4B | TSG |
| NFKBIE | nuclear factor of kappa light polypeptide gene enhancer in B-cells | TSG |
| | inhibitor, epsilon | |
| PHOX2B | paired-like homeobox 2b | TSG |
| PIK3R1 | phosphoinositide-3-kinase, regulatory subunit 1 (alpha) | TSG |
| PTPN13 | protein tyrosine phosphatase, non-receptor type 13 | TSG |
| SDHAF2 | succinate dehydrogenase complex assembly factor 2 | TSG |
| SETD1B | SET domain containing 1B | TSG |
| TENT5C | Terminal nucleotidyltransferase 5C | TSG |
| TGFBR2 | transforming growth factor beta receptor II | TSG |
| CNTNAP2 | contactin associated protein like 2 | TSG |
| IGF2BP2 | insulin like growth factor 2 mRNA binding protein 2 | TSG |
| PPP2R1A | protein phosphatase 2, regulatory subunit A, alpha | TSG |
| SMARCA4 | SWI/SNF related, matrix associated, actin dependent regulator of | TSG |
| | chromatin, subfamily a, member 4 | |
| SMARCB1 | SWI/SNF related, matrix associated, actin dependent regulator of | TSG |
| | chromatin, subfamily b, member 1 | |
| SMARCD1 | SWI/SNF related, matrix associated, actin dependent regulator of | TSG |
| | chromatin, subfamily d, member 1 | |
| SMARCE1 | SWI/SNF related, matrix associated, actin dependent regulator of | TSG |
| | chromatin, subfamily e, member 1 | |
| TMEM 127 | transmembrane protein 127 | TSG |
| TNFAIP3 | tumor necrosis factor, alpha-induced protein 3 | TSG |
| ARHGAP35 | Rho GTPase activating protein 35 | TSG |
| ARHGEF10 | Rho guanine nucleotide exchange factor 10 | TSG |
| LEPROTL1 | leptin receptor overlapping transcript like 1 | TSG |
| TNFRSF14 | tumor necrosis factor receptor superfamily, member 14 | TSG |
| | (herpesvirus entry mediator) | |
| ARHGEF10L | Rho guanine nucleotide exchange factor 10 like | TSG |
| ELL | ELL gene (11-19 lysine-rich leukemia gene) | TSG, fusion |
| FUS | fusion, derived from t(12; 16) malignant liposarcoma | TSG, fusion |
| NF1 | neurofibromatosis type 1 gene | TSG, fusion |
| PML | promyelocytic leukemia | TSG, fusion |
| ABI1 | abl-interactor 1 | TSG, fusion |
| BCOR | BCL6 corepressor | TSG, fusion |
| BTG1 | B-cell translocation gene 1, anti-proliferative | TSG, fusion |
| CARS | cysteinyl-tRNA synthetase | TSG, fusion |
| CBFB | core-binding factor, beta subunit | TSG, fusion |
| CDX2 | caudal type homeo box transcription factor 2 | TSG, fusion |
| CLTC | clathrin, heavy polypeptide (Hc) | TSG, fusion |
| CNBP | CCHC-type zinc finger, nucleic acid binding protein | TSG, fusion |
| EBF1 | early B-cell factor 1 | TSG, fusion |
| ETV6 | ets variant gene 6 (TEL oncogene) | TSG, fusion |
| EXT1 | multiple exostoses type 1 gene | TSG, fusion |
| FHIT | fragile histidine triad gene | TSG, fusion |
| KNL1 | cancer susceptibility candidate 5 | TSG, fusion |
| MLF1 | myeloid leukemia factor 1 | TSG, fusion |
| MYH9 | myosin, heavy polypeptide 9, non-muscle | TSG, fusion |
| NAB2 | NGFI-A binding protein 2 | TSG, fusion |
| NRG1 | neuregulin 1 | TSG, fusion |
| PER1 | period homolog 1 (Drosophila) | TSG, fusion |
| RHOH | ras homolog family member H | TSG, fusion |
| RMI2 | RecQ mediated genome instability 2 | TSG, fusion |
| SFPQ | splicing factor proline/glutamine rich(polypyrimidine tract binding | TSG, fusion |
| | protein associated) | |
| TPM3 | tropomyosin 3 | TSG, fusion |
| WIF1 | WNT inhibitory factor 1 | TSG, fusion |
| BCL10 | B-cell CLL/lymphoma 10 | TSG, fusion |
| CCDC6 | coiled-coil domain containing 6 | TSG, fusion |
| CD274 | CD274 molecule | TSG, fusion |
| CDH11 | cadherin 11, type 2, OB-cadherin (osteoblast) | TSG, fusion |
| CIITA | class II, major histocompatibility complex, transactivator | TSG, fusion |
| DDX10 | DEAD (Asp-Glu-Ala-Asp) box polypeptide 10 | TSG, fusion |
| EIF3E | eukaryotic translation initiation factor 3, subunit E | TSG, fusion |
| EP300 | 300 kd E1A-Binding protein gene | TSG, fusion |
| EPS15 | epidermal growth factor receptor pathway substrate 15 (AF1p) | TSG, fusion |
| IKZF1 | IKAROS family zinc finger 1 | TSG, fusion |
| KAT6B | K(lysine) acetyltransferase 6B | TSG, fusion |
| LRIG3 | leucine-rich repeats and immunoglobulin-like domains 3 | TSG, fusion |
| NCOA4 | nuclear receptor coactivator 4 - PTC3 (ELE1) | TSG, fusion |
| NDRG1 | N-myc downstream regulated 1 | TSG, fusion |
| PATZ1 | zinc finger protein 278 (ZSG) | TSG, fusion |
| PPARG | peroxisome proliferative activated receptor, gamma | TSG, fusion |
| PTPRK | protein tyrosine phosphatase, receptor type, K | TSG, fusion |
| RPL22 | ribosomal protein L22 (EAP) | TSG, fusion |
| RSPO2 | R-spondin 2 | TSG, fusion |
| YWHAE | tyrosine 3-monooxygenase/tryptophan 5-monooxygenase | TSG, fusion |
| | activation protein, epsilon polypeptide (14-3-3 epsilon) | |
| ARID1A | AT rich interactive domain 1A (SWI-like) | TSG, fusion |
| CAMTA1 | calmodulin binding transcription activator 1 | TSG, fusion |
| CLTCL1 | clathrin, heavy polypeptide-like 1 | TSG, fusion |
| RAD51B | RAD51 paralog B | TSG, fusion |
| RANBP2 | RAN binding protein 2 | TSG, fusion |
| TRIM33 | tripartite motif-containing 33 (PTC7, TIF1G) | TSG, fusion |
| ZBTB16 | zinc finger and BTB domain containing 16 | TSG, fusion |
| ZNF331 | zinc finger protein 331 | TSG, fusion |
| CBFA2T3 | core-binding factor, runt domain, alpha subunit 2; translocated to, 3 | TSG, fusion |
| | (MTG-16) | |
| CREB3L1 | cAMP responsive element binding protein 3-like 1 | TSG, fusion |
| SLC34A2 | solute carrier family 34 (sodium phosphate), member 2 | TSG, fusion |
| ARHGAP26 | Rho GTPase activating protein 26 | TSG, fusion |
| ARHGEF12 | RHO guanine nucleotide exchange factor (GEF) 12 (LARG) | TSG, fusion |
| CCNB1IP1 | cyclin B1 interacting protein 1, E3 ubiquitin protein ligase | TSG, fusion |
| DCC | DCC netrin 1 receptor | |
| ISX | intestine specific homeobox | |
| ANK1 | ankyrin 1 | |
| BMP5 | bone morphogenetic protein 5 | |
| FAT3 | FAT atypical cadherin 3 | |
| FLNA | filamin A | |
| NBEA | neurobeachin | |
| PMS1 | PMS1 postmeiotic segregation increased 1 (S. cerevisiae) | |
| RGS7 | regulator of G protein signaling 7 | |
| CD209 | CD209 molecule | |
| CNBD1 | cyclic nucleotide binding domain containing 1 | |
| ECT2L | epithelial cell transforming sequence 2 oncogene-like | |
| EPHA3 | EPH receptor A3 | |
| EPHA7 | EPH receptor A7 | |
| FKBP9 | FK506 binding protein 9 | |
| ITGAV | integrin subunit alpha V | |
| PCBP1 | poly(rC) binding protein 1 | |
| PRKCB | protein kinase C beta | |
| RGPD3 | RANBP2-like and GRIP domain containing 3 | |
| BCLAF1 | BCL2 associated transcription factor 1 | |
| CRNKL1 | crooked neck pre-mRNA splicing factor 1 | |
| CTNND1 | catenin delta 1 | |
| CYP2C8 | cytochrome P450 family 2 subfamily C member 8 | |
| EIF1AX | eukaryotic translation initiation factor 1A, X-linked | |
| FAM47C | family with sequence similarity 47 member C | |
| MB21D2 | Mab-21 domain containing 2 | |
| ZNF429 | zinc finger protein 429 | |
| ZNF479 | zinc finger protein 479 | |
| FAM135B | family with sequence similarity 135 member B | |
| PRPF40B | pre-mRNA processing factor 40 homolog B | |
| DCAF12L2 | DDB1 and CUL4 associated factor 12 like 2 | |

The effectors described herein can also be used to conduct screens for identifying small molecule binders such as PROTACs, molecular glues and other heterobifunctional molecules.

Accordingly, another aspect includes a screening assay for identifying a ligand optionally a small molecule binder of at least one recombinant proximity effector polypeptide, the screening assay comprising:
contacting the or the at least one recombinant proximity effector polypeptide with a small molecule library optionally in a high-throughput screening assay, wherein the proximity effector polypeptide is selected from Table 4, 5, 6 or 7;
assessing whether binding has occurred between the recombinant proximity effector polypeptide and one or more small molecule(s) of the small molecule library,
   wherein the one or more molecule(s) which have bound to the or the at least one recombinant proximity effector polypeptide is a small molecule binder of the or the at least one recombinant proximity effector polypeptide.

The ligand as used herein means any compound or composition of matter, including small molecules, for example molecules with a molecular weight of less than or equal to about 1000 Da.

In some embodiments, the ligand optionally the small molecule binder is a PROTAC. In some embodiments, the ligand, optionally small molecule binder is a molecular glue.

In some embodiments, the method further comprises making a product optionally a therapeutic product.

Any of the effectors and fusion polypeptides group or subgroup described herein can be used. The recombinant polypeptide can be made using various expression systems, including bacterial, mammalian, or insect systems. They can be fused with a signal peptide so that they are secreted allowing for ease of purification.

Any of the methods described herein, including those described in Example 6 can be used.

The methods, processes and/or screening assays can be combined. For Example, a method of identifying a proximity effector polypeptide as described herein can be performed. An effector can be selected and subjected to a screening assay described herein to identify a a small molecule binder. Also provided in another aspect, are methods of making a heterobifunctional molecule, the method comprising coupling ligand such as a small molecule binder of a proximity effector polypeptide and a ligand such as a small molecule binder of a target polypeptide via a linker, optionally a linker described herein.

The identified ligand can be used for the preparation of a product optionally a therapeutic product. In some embodiments, the method further comprises making a product optionally a therapeutic product.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Materials and Methods

### Cell Lines

HeLa Kyoto cell and all HEK293T cell lines, including the ABI1-EGFP-IRES-TagBFP reporter cell line used for screens, were maintained in DMEM supplemented with 10% fetal bovine serum (FBS) and 1% penicillin-streptomycin. Cells were maintained at 37° C. in a humidified incubator at 5% CO2 and routinely tested for Mycoplasma contamination. Lentivirus production

Lentiviral particles containing the pooled ORFeome were produced by transfecting 293T cells with pLX301-[ORF]-PYL1 or pLX301-[ORF]-vhhGFP, psPAX2 (Addgene #12260) and pVSV-G (Addgene #8454) at a ratio of 8:8:1. Transfection was performed using Lipofectamine 2000 (Thermo Fisher Scientific, 11668019) on 15-cm dishes according to the manufacturer's protocol. The medium was changed 24 hours post-transfection. 72 hours after transfection, supernatant was filtered (0.45 μM), pooled and collected. A similar protocol was followed for small scale virus production when establishing individual stable cell lines with transfection being performed on 6-well plates using lipofectamine 2000 reagent.

### Cell Lines Generation

A clonal line of the ABI1-EGFP reporter line was generated expressing ABI1-EGFP-IRES-TagBFP (blasticidin, 6 pg/mL). Single cells were sorted and expanded and a clone showing high EGFP and TagBFP expression was selected for subsequent experiments. To generate 293T cells expressing doxycycline-inducible EGFP tagged proteins, entry cloned were picked from the hORFeome collection and subcloned into the Gateway compatible pSTV6-TetO-ccdB-EGFP lentiviral plasmid. 293T cells were infected at the presence of 8 pg/mL polybrene and selected with 2 μg/mL puromycin 24 hours post infection. EGFP cell lines were induced with 1 μg/mL doxycycline and sorted (BD FACS Melody) for the highest GFP population.

### Plasmids Cloning

Unstable mutant targets were cloned into pcDNA3.1-ccdB-GSlinker-EGFP-P2A-DsRed destination vector, using gateway cloning technology.

For the degradation assay, effectors were cloned into pcDNA3.1-ccdB-GSlinker-vhhGFP-SV40-TagBFP, pcDNA3.1-ccdB-GSlinker-vhhGFP, pcDNA3.1-vhhGFP-ccdB and pcDNA3.1-ccdB-GSlinker-PYL1 destination vectors.

For WDR5 endogenous protein expression, effector-coding sequences were cloned into the pcDNA3.4-ccdB-Mb(S4) WDR5-HA vector, allowing expression of the respective proteins with a C-terminal monobody recognizing WDR5 with a high affinity (Gupta et al., 2018).

For KRas exogenous protein expression, KRas was cloned into pcDNA3.1-3×FLAG-ccdB destination vector. The effector-coding sequences were cloned into the pcDNA3.4-ccdB-iDab-KRas-HA and the pcDNA3.1-ccdB-iDab-LMO2-HA vectors, allowing expression of the respective proteins with a C-terminal monobody recognizing KRas or another protein, respectively (Tanaka et al., 2007 and 2011). The effectors were also cloned into the pcDNA3.4-ccdB-DARPinK19-HA and the pcDNA3.1-ccdB-DARPinK19mutated-HA vectors, allowing expression of the respective proteins with a C-terminal monobody selectively binding KRas or a mutated monobody losing the interaction, respectively (Bery et al., 2019).

Quick-change site-directed mutagenesis was used for production of point-mutations (UBE2B, FCGR3B and PRPS2 mutants) using standard PCR practices.

### Pooled ORFeome Library Generation

Entry clones from the human ORFeome collection (v8.1) were collected into 40 standardized subpools each containing ˜384 ORFs and cloned into the lentiviral Gateway-compatible destination vector pLX301-DEST-PYL1 or pLX301-DEST-vhhGFP. LR reactions were set up in duplicates with 150 ng of each entry ORF subpool, combined with 1 μl of Gateway LR clonase II in a total of 5 μl reaction volume and incubated overnight in TE buffer at room temperature. For the next two days, 1 μl additional LR enzyme was added in 4 μl TE and 150 ng destination vector to each reaction. Subpools were transformed into chemically competent Stbl3 E. coli and spread on LB agar plates containing ampicillin (100 μg/μl) overnight at 30° C. Colonies were counted to ensure >200-fold coverage, collected in SOC on ice, pelleted and maxiprepped on multiple columns based on weight of the dry pellets.

### Pooled Activation Screens

ORFeome libraries tagged at the C-terminus with PYL1 or vhhGFP were packaged into lentiviral particles. A clonal GFP reporter cell line stably co-expressing ABI1-GFP was transduced at low multiplicity of infection (MOI) with approximately 30% cell survival after puromycin (1 μg/mL) selection. Untransduced cells under the same condition were fully eliminated. Sufficient cells were transduced to maintain >500 fold coverage of the libraries. For the ORFeome-PYL1 library, recruitment was induced by treating cells with 100 μM abscisic acid (ABA, Sigma) for 48 hours. In parallel, a control batch of cells were treated with equal total volume of DMSO. Cells were then washed in PBS, treated with dissociation buffer (1 mM EDTA, 10 mM KCl, 150 mM NaCl, 5 mM sodium bicarbonate, 0.1% glucose) and resuspended in flow buffer (5 mM EDTA, 25 mM HEPES pH 7, 1% BSA, PBS). For each library, high GFP (top 10%) and low GFP (bottom 10%) populations were sorted (in duplicate) using BD FACS Melody (Stagljar lab, CCBR) and their genomic DNA directly extracted using QIAmp DNA Blood Mini Kit (QIAGEN).

### ORFeome Sequencing

Nested PCR was performed using all the purified genomic DNA from sorted populations or at least 5 μg of genomic DNA from unsorted populations. The target ORFeome region was amplified from genomic DNA using primers targeting the T7 promoter (SEQ ID NO 1: CGACTCACTATAGGGAGACCCAAG) and PYL1 (SEQ ID NO 2: ATTCATCTTGCGTTGGTGCTCC) or vhhGFP (SEQ ID NO 3: GCCACCAGACTCCACCAGTTGGAC). The product of this reaction was pooled for each sample and further amplified by primers targeting outside the Gateway attB sites (SEQ ID NO 4: CAGTGTGGTGGAATTCTGCAG and SEQ ID NO 5: CCGCCACTGTGCTGGATATC) for an additional 10 cycles. Amplicons were subsequently separated on 1% agarose gel and any visible PCR product excluding primer dimers were gel purified. After quantifying DNA using the Quant-iT 1× dsDNA HS kit (Thermo Fisher Scientific, Q33232), 50 ng per sample was processed using the Illumina DNA Prep, (M) Tagmentation kit (Illumina, 20018705), with 6 cycles of amplification. 2 μl of each purified final library was run on an Agilent TapeStation HS D1000 ScreenTape (Agilent Technologies, 5067-5584). The libraries were quantified using the Quant-iT 1× dsDNA HS kit (Thermo Fisher Scientific, Q33232) and pooled at equimolar ratios after size-adjustment. The final pool was quantified using NEBNext Library Quant Kit for Illumina (New England Biolabs, E7630L) and paired-end sequenced on an Illumina MiSeq.

Analysis of Sequencing Data from Pooled Activation Screens

An index of the ORFeome reference sequences was created using the STAR aligner v2.7.8a. Reads from the ORFeome libraries were aligned with the STAR aligner allowing a maximum of 3 mismatches. To identify degraders and stabilizers, the edgeR package (Robinson et al., 2010) was used to calculate log 2 fold change, p-value, and false discovery rate (FDR) for each ORF by comparing changes in counts from sorted samples to unsorted cells.

### Degradation Assay for Individual Effectors

Degradation assays with individual effectors were performed in a 48-well cell culture format by transient transfection of the clonal GFP reporter cell line stably co-expressing ABI1-GFP with 15 ng of transfection control plasmid expressing triple FLAG-tagged DsRed and 200 ng of the effector fused to vhhGFP or PYL1, using lipofectamine 2000 (Life Technologies). For the unstable mutant stabilization experiments, 293T were co-transfected with 100 ng of the effector fused to vhhGFP and 100 ng of the target fused to GFP. For the effector-PYL1 constructs, recruitment was induced by treating cells with 100 μM abscisic acid (ABA, Sigma) for 48 hours. In parallel, a control batch of cells were treated with equal total volume of DMSO. 48 hours post-transfection, cells were washed in PBS, treated with dissociation buffer and resuspended in flow buffer. Cells were spun down in a microcentrifuge at 1000 rpm for 5 minutes. Cell pellets were resuspended in flow buffer and analyzed using BD LSR Fortessa or BD LSR Fortessa X20 (BD Biosciences; University of Toronto Faculty of Medicine Flow Cytometry Facility).

### Westernblot

HeLa cells (in 24-well plate) were transfected with 0.8 μg of effector fused to Mb(S4) WDR5 monobody. HeLa cells (in 12-well plate) were transfected with 0.4 μg of 3×FLAG-KRas and 0.4μg of effector fused to iDab or DARPin K-RAS nanobody. 48h hours after transfection, cells were harvested and lysed in 50 μL of CKS lysis buffer (20 mM Hepes-KOH pH 7.9, 100 mM NaCl, 1 mM MgCl2, 1 mM EDTA, 300 mM sucrose, 1 mM DTT, 0.1% Triton X-100, benzonase and proteases inhibitor cocktail). After centrifugation at 16 000 g for 5 min at 4° C., the cellular lysates were analyzed by gel electrophoresis and western blot using anti-WDR5 (D9E1 I) antibody (Cell Signaling #13105), anti-HSP90a/b (F-8) antibody (Santa Cruz Biotechnology), and anti-HA antibody (Sigma H3663) as primary antibody. Goat HRP-conjugated anti-rabbit IgG (Cell Signaling #7074S) or anti-mouse (Cell Signaling #7076S) were used as secondary antibody.

### Inhibitor Treatments

Cells were treated with 1 μM MLN4924 (Chemiteck) for 24 hours, 100 nM Bortezomib (Calbiochem) for 6 hours, 20 μM cycloheximide (Sigma) for 6 hours, 2.5 μM CB-5083 (Selleckchem) for 6 hours or 0.01% DMSO (Fisher bioreagents) for 6 or 24 hours.

### Results

Using a functional proteomics screen, a collection of human proteins that degrade or stabilize other proteins highly efficiently in a proximity-dependent manner have been identified. These proteins could be harnessed for induced proximity therapeutics, such as targeted protein degradation (TPD) or targeted protein stabilization, using heterobifunctional molecules (e.g. PROTACs) or molecular glues. The proteins identified have features that make them appealing to such development, including insensitivity to induced complex geometry and potency across multiple targets and compartments in the cell.

### ORFeome-Wide Induced Proximity Screen for Protein Stability Effectors

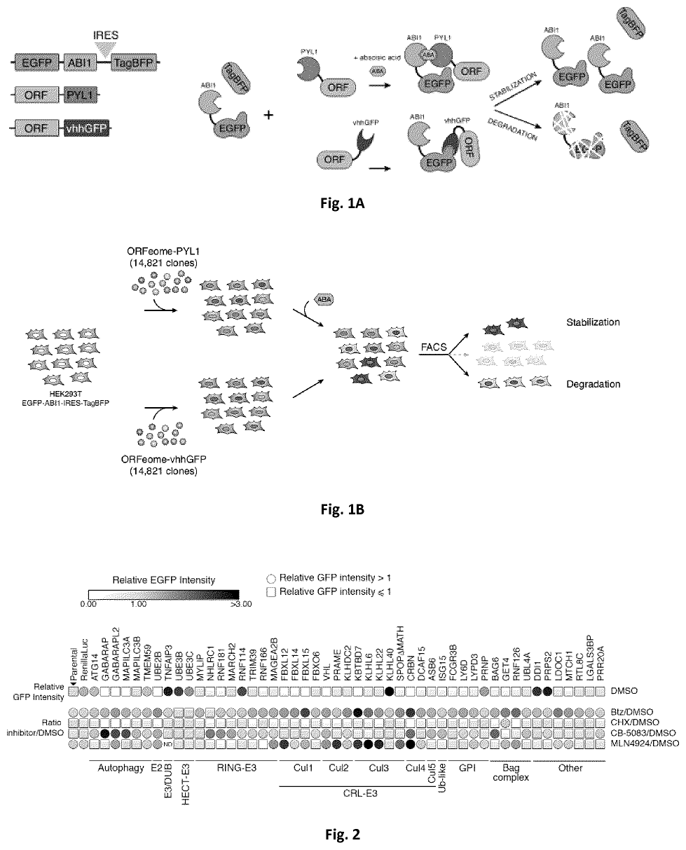

An unbiased approach to discover proximity-dependent effectors at proteome scale was developed. It involves co-expressing a target and an effector protein in cells with tags that allow for induction of their interaction. As a proof-of-principle, proteins that degrade or stabilize a GFP fusion protein in a proximity-dependent manner were identified. A stable 293T cell line expressing an ABI1-GFP fusion followed by an internal ribosome entry site (IRES) and BFP (FIG. 1) was generated. This cell line was then transduced with an ORFeome-derived lentiviral pooled library expressing 18,937 open reading frames (ORFs) fused to either vhhGFP, a nanobody that binds to GFP (Caussinus et al., 2012; Saerens et al., 2005), or to PYL1, a domain that binds ABI1 in the presence of abscisic acid (ABA)(Liang et al., 2011). By this design, each protein in the ORFeome could be brought in proximity to GFP-ABI1 either constitutively (vhhGFP) or by chemical dimerization (PYL1). Sorting cells with low or high GFP/BFP ratio followed by ORF sequencing identified proteins that degrade or stabilize GFP-ABI1, respectively (FIG. 1B). Unexpectedly, there were many proteins that were not regulators of ubiquitination, autophagy, or lysosomal degradation, suggesting that the proteome has a previously uncharacterized cache of proximity-dependent regulators of protein stability. Effector proteins identified as degrading or stabilizing GFP-ABI1 are included in Table 4.

TABLE 4
C-terminal fusion effector polypeptides identified as degrading or stabilizing GFP-ABI1

| | UniProt | | | | UniProt | | |
|---|---|---|---|---|---|---|---|
| | Accession | | | | Accession | | |
| | Number or | Degrader/ | | | Number or | Degrader/ | |
| Protein | SEQ ID NO | Stabilizer | E3 ligase type | Protein | SEQ ID NO | Stabilizer | E3 ligase type |
| ASB3 | Q9Y575 | Degrader | E3 (CRL adaptor) | HPRT1 | P00492 | Stabilizer | |
| ASB6 | Q9NWX5 | Degrader | E3 (CRL adaptor) | HSD17B1 | P14061 | Stabilizer | |
| ATG14 | Q6ZNE5 | Stabilizer | | HSD17B14 | Q9BPX1 | Stabilizer | |
| BAG6 | P46379 | Degrader | | HSPA12A | O43301 | Stabilizer | |
| CRBN | Q96SW2 | Degrader | E3 (CRL adaptor) | HSPA1A | P0DMV8 | Stabilizer | |
| DCAF15 | Q66K64 | Degrader | E3 (CRL adaptor) | HSPA1B | P0DMV9 | Stabilizer | |
| DDI1 | Q8WTUO | Stabilizer | | HTRA3 | P83110 | Degrader | |
| DNAJC14 | Q6Y2X3 | no effect | | HYAL3 | O43820 | Degrader | |
| EID1 | Q9Y6B2 | Degrader | | ICAM2 | P13598 | Stabilizer | |
| FBXL12 | Q9NXK8 | Degrader | E3 (CRL adaptor) | IDH2 | P48735 | Degrader | |
| FBXL14 | Q8N1E6 | Degrader | E3 (CRL adaptor) | IFRG15 | Q8NFQ8 | Degrader | E3 (CRL adaptor) |
| FBXL15 | Q9H469 | Degrader | E3 (CRL adaptor) | IGF2BP2 | Q9Y6M1 | Stabilizer | |
| FBXO6 | Q9NRD1 | Degrader | E3 (CRL adaptor) | IGHM | P01871 | Stabilizer | |
| FCGR3B | O75015 | Degrader | | IGSF1 | Q8N6C5 | Degrader | |
| FOLR2 | P14207 | Degrader | | IGSF21 | Q96ID5 | Degrader | |
| GABARAP | O95166 | Degrader | | IL1RN | P18510 | Stabilizer | |
| GABARAPL2 | P60520 | Degrader | | IL21R | Q9HBE5 | Degrader | |
| GET4 | Q7L5D6 | Degrader | | IMPDH1 | P20839 | Stabilizer | |
| GID8 | Q9NWU2 | Stabilizer | | IRAK1BP1 | Q5VVH5 | Degrader | |
| ISG15 | P05161 | Degrader | | ISLR | O14498 | Degrader | |
| KBTBD7 | Q8WVZ9 | Degrader | E3 (CRL adaptor) | ISYNA1 | Q9NPH2 | Stabilizer | |
| KLHDC2 | Q9Y2U9 | Degrader | E3 (CRL adaptor) | ITFG3 | Q9H0X4 | Stabilizer | |
| KLHL22 | Q53GT1 | Degrader | E3 (CRL adaptor) | IZUMO1 | Q8IYV9 | Degrader | |
| KLHL40 | Q2TBA0 | Stabilizer | E3 (CRL adaptor) | KATNB1 | Q9BVA0 | Degrader | E3 (CRL adaptor) |
| KLHL41 | O60662 | Stabilizer | E3 (CRL adaptor) | KBTBD5 | Q2TBA0 | Stabilizer | |
| KLHL6 | Q8WZ60 | Degrader | E3 (CRL adaptor) | KCNA1 | Q09470 | Stabilizer | |
| LDOC1 | O95751 | Degrader | | KCND1 | Q9NSA2 | Stabilizer | |
| LGALS3BP | Q08380 | Degrader | | KCND3 | Q9UK17 | Stabilizer | |
| LY6D | Q14210 | Degrader | | KCNJ15 | Q99712 | Degrader | |
| LYPD3 | O95274 | Degrader | | KCNJ5 | P48544 | Stabilizer | |
| MAGEA2B | P43356 | Degrader | RING-E3 | KCNJ6 | P48051 | Stabilizer | |
| MAP1LC3A | Q9H492 | Degrader | | KCNK1 | O00180 | Stabilizer | |
| MAP1LC3B | Q9GZQ8 | Degrader | | KCNK6 | Q9Y257 | Stabilizer | |
| MARCH2 | Q9P0N8 | Degrader | RING-E3 | KCTD12 | Q96CX2 | Stabilizer | |
| MTCH1 | Q9NZJ7 | Degrader | | KCTD16 | Q68DU8 | Degrader | E3 (CRL adaptor) |
| MYLIP | Q8WY64 | Degrader | RING-E3 | KHK | P50053 | Stabilizer | |
| NHLRC1 | Q6VVB1 | Degrader | RING-E3 | KIAA1715 | Q9C0E8 | Stabilizer | |
| OTUB1 | Q96FW1 | Stabilizer | | KLF4 | O43474 | Degrader | |
| PLEKHB2 | Q96CS7 | no effect | | KLHL11 | Q9NVR0 | Degrader | E3 (CRL adaptor) |
| PRAME | P78395 | Degrader | E3 (CRL adaptor) | KLHL13 | Q9P2N7 | Degrader | E3 (CRL adaptor) |
| PRNP | P04156 | Stabilizer | | KLHL23 | Q8NBE8 | Degrader | E3 (CRL adaptor) |
| PRPS2 | P11908 | Stabilizer | | KLHL3 | Q9UH77 | Degrader | E3 (CRL adaptor) |
| PRR20A | P86496 | Degrader | | KRT8 | P05787 | Stabilizer | |
| RNF114 | Q9Y508 | Stabilizer | RING-E3 | LAPTM4B | Q86VI4 | Degrader | |
| RNF126 | Q9BV68 | Degrader | RING-E3 | LAPTM5 | Q13571 | Degrader | |
| RNF166 | Q96A37 | Degrader | RING-E3 | LDHB | P07195 | Stabilizer | |
| RNF181 | Q9P0P0 | Degrader | RING-E3 | LGALS1 | P09382 | Stabilizer | |
| RTL8C | A6ZKI3 | Degrader | | LGALS4 | P56470 | Stabilizer | |
| SPOP | O43791 | Degrader | E3 (CRL adaptor) | LIN28A | Q9H9Z2 | Stabilizer | |
| TEX19 | Q8NA77 | Degrader | | LINGO1 | Q96FE5 | Stabilizer | |
| TMEM204 | Q9BSN7 | Degrader | | LIPG | Q9Y5X9 | Degrader | |
| TMEM59 | Q9BXS4 | no effect | | LIPH | Q8WWY8 | Degrader | |
| TNFAIP3 | P21580 | Stabilizer | E3/DUB | LMAN2L | Q9H0V9 | Degrader | |
| TRIM39 | Q9HCM9 | Degrader | RING-E3 | LMNA | P02545 | Stabilizer | |
| UBE2A | P49459 | Degrader | | LNX2 | Q8N448 | Degrader | RING-E3 |
| UBE2B | P63146 | Degrader | | LONRF1 | Q17RB8 | Degrader | RING-E3 |
| UBE2C | O00762 | Degrader | | LRAT | O95237 | Degrader | |
| UBE2D1 | P51668 | Degrader | | LRRC14B | A6NHZ5 | Degrader | |
| UBE2D3 | P61077 | Degrader | | LRRC28 | Q86X40 | Degrader | |
| UBE2D4 | Q9Y2X8 | Degrader | | LRRC56 | Q8IYG6 | Stabilizer | |
| UBE2DNL | Q8IWF7 | Degrader | | LRRC57 | Q8N9N7 | Stabilizer | |
| UBE2E2 | Q96LR5 | no effect | | LTC4S | Q16873 | Degrader | |
| UBE2E3 | Q969T4 | no effect | | MACROD1 | Q9BQ69 | Stabilizer | |
| UBE2F | Q969M7 | no effect | | MAGEA4 | P43358 | Stabilizer | |
| UBE2G1 | P62253 | Stabilizer | | MAPK12 | P53778 | Stabilizer | |
| UBE2G2 | P60604 | Degrader | | MAPK13 | O15264 | Stabilizer | |
| UBE2H | P62256 | no effect | | MARCHF10 | Q8NA82 | Stabilizer | |
| UBE2I | P63279 | Degrader/ | | MARCHF2 | Q9P0N8 | Degrader | RING-E3 |
| | | Stabilizer (2 | | | | | |
| | | isoforms) | | | | | |
| UBE2J2 | Q8N2K1 | Degrader | | MARCKSL1 | P49006 | Stabilizer | |
| UBE2K | P61086 | no effect | | MB | P02144 | Stabilizer | |
| UBE2L3 | P68036 | Stabilizer | | MED24 | O75448 | Degrader | |
| UBE2L6 | O14933 | Stabilizer | | METTL21A | Q8WXB1 | Stabilizer | |
| UBE2M | P61081 | Stabilizer | | METTL2A | Q96IZ6 | Degrader | |
| UBE2N | P61088 | Degrader | | MGLL | Q99685 | Stabilizer | |
| UBE2O | Q9C0C9 | Stabilizer | | MIF | P03971 | Stabilizer | |
| UBE2Q1 | Q7Z7E8 | Degrader | | MKRN1 | Q9UHC7 | Degrader | |
| UBE2Q2 | Q8WVN8 | no effect | | MLXIP | Q9HAP2 | Degrader | |
| UBE2R2 | Q712K3 | Stabilizer | | MLYCD | O95822 | Stabilizer | |
| UBE2S | Q16763 | no effect | | MMGT1 | Q8N4V1 | Degrader | |
| UBE2T | Q9NPD8 | Degrader | | MMP15 | P51511 | Degrader | |
| UBE2U | Q5VVX9 | Degrader | | MND1 | Q9BWT6 | Degrader | |
| UBE2V1 | Q13404 | Stabilizer | | MOB3C | Q70IA8 | Stabilizer | |
| UBE2V2 | Q15819 | Stabilizer | | MPP1 | Q00013 | Degrader | |
| UBE2W | Q96B02 | Degrader | | MRAP | Q8TCY5 | Degrader | |
| UBE2Z | Q9H832 | Degrader | | MRAS | O14807 | Degrader | |
| UBE3B | Q7Z3V4 | Stabilizer | HECT-E3 | MRFAP1L1 | Q96HT8 | Degrader | |
| UBE3C | Q15386 | Stabilizer | HECT-E3 | MRGPRF | Q96AM1 | Stabilizer | |
| UBL4A | P11441 | Degrader | | MRPS34 | P82930 | Degrader | |
| UCHL1 | P09936 | Stabilizer | | MRTO4 | Q9UKD2 | Stabilizer | |
| VHL | P40337 | Degrader | E3 (CRL adaptor) | MS4A4A | Q96JQ5 | Degrader | |
| KLHL40 | KLHL40 | Degrader | E3 (CRL adaptor) | MTHFS | P49914 | Stabilizer | |
| Fusion | (Q2TBA0) | | | | | | |
| Polypeptide | with BTB | | | | | | |
| | domain of | | | | | | |
| | KLHL6 | | | | | | |
| | (Q8WZ60) | | | | | | |
| KLHL6 | KLHL6 | Stabilizer | E3 (CRL adaptor) | MTMR14 | Q8NCE2 | Stabilizer | |
| Fusion | (Q8WZ60) | | | | | | |
| Polypeptide | with BTB | | | | | | |
| | domain of | | | | | | |
| | KLHL40 | | | | | | |
| | (Q2TBA0) | | | | | | |
| PRNP | PRNP | Degrader | | MTPN | P58546 | Stabilizer | |
| Fusion | (P04156) | | | | | | |
| Polypeptide | with the C- | | | | | | |
| | terminal | | | | | | |
| | residues | | | | | | |
| | 194-223 of | | | | | | |
| | FCGR3B | | | | | | |
| | (O75015-1) | | | | | | |
| DDI1- | DDI1 | Stabilizer | | MTSS1L | Q765P7 | Stabilizer | |
| Fragment 1 | (Q8WTU0) | | | | | | |
| | without RVP | | | | | | |
| | domain | | | | | | |
| DDI1 - | DDI1 | Stabilizer | | MVD | P53602 | Stabilizer | |
| Fragment 2 | (Q8WTU0) | | | | | | |
| | RVP domain | | | | | | |
| | only | | | | | | |
| DDI1 - | DDI1 | Stabilizer | | MVK | Q03426 | Stabilizer | |
| Fragment 3 | (Q8WTU0) | | | | | | |
| | without UBL | | | | | | |
| | domain | | | | | | |
| DDI1 - | DDI1 | Stabilizer | | MYD88 | Q99836 | Stabilizer | |
| Fragment 4 | (Q8WTU0) | | | | | | |
| | without HDD | | | | | | |
| | domain | | | | | | |
| Mutant | P11908 with | Stabilizer | | MYL4 | P12829 | Stabilizer | |
| PRSP2 | Q133P | | | | | | |
| Q133P | mutation | | | | | | |
| Mutant | P11908 with | Stabilizer | | MYL9 | P24844 | Degrader | |
| PRSP2 | H130A | | | | | | |
| H130A | mutation | | | | | | |
| KLHL40 | Q2TBA0 - | Stabilizer | E3 (CRL adaptor) | NAA50 | Q9GZZ1 | Degrader | |
| Fragment 1 | only BTB | | | | | | |
| | and BACK | | | | | | |
| | domains | | | | | | |
| KLHL40 | Q2TBA0 | Stabilizer | E3 (CRL adaptor) | NADK | O95544 | Stabilizer | |
| Fragment 2 | without | | | | | | |
| | KELCH | | | | | | |
| | repeats | | | | | | |
| KLHL40 | Q2TBA0 | Degrader | E3 (CRL adaptor) | NAP1L5 | Q96NT1 | Stabilizer | |
| Fragment 3 | without BTB | | | | | | |
| | domain | | | | | | |
| KLHL40 | Q2TBA0 | Stabilizer | E3 (CRL adaptor) | NCALD | P61601 | Stabilizer | |
| Fragment 4 | without | | | | | | |
| | BACK | | | | | | |
| | domain | | | | | | |
| KLHL40 | Q2TBA0 | Stabilizer | E3 (CRL adaptor) | NCCRP1 | Q6ZVX7 | Stabilizer | |
| Fragment 5 | without | | | | | | |
| | residues 1- | | | | | | |
| | 10 | | | | | | |
| FBXO3 | Q9UK99 | Degrader | E3 (CRL adaptor) | NCF2 | P19878 | Stabilizer | |
| FBXO40 | Q9UH90 | Degrader | E3 (CRL adaptor) | NCK2 | O43639 | Stabilizer | |
| FBXW5 | Q969U6 | Degrader | E3 (CRL adaptor) | NCS1 | P62166 | Stabilizer | |
| BTRC | Q9Y297 | Degrader | E3 (CRL adaptor) | NDFIP2 | Q9NV92 | Degrader | |
| CISH | Q9NSE2 | Degrader | E3 (CRL adaptor) | NDRG1 | Q92597 | Stabilizer | |
| SOCS5 | O75159 | Degrader | E3 (CRL adaptor) | NECAP2 | Q9NVZ3 | Stabilizer | |
| GMCL1 | Q96IK5 | Degrader | E3 (CRL adaptor) | NIPAL4 | Q0D2K0 | Degrader | |
| KLHL12 | Q53G59 | Degrader | E3 (CRL adaptor) | NKX2-1 | P43699 | Degrader | |
| GAN | Q9H2C0 | Degrader | E3 (CRL adaptor) | NME1 | P15531 | Stabilizer | |
| KBTBD2 | Q8IY47 | Degrader | E3 (CRL adaptor) | NPDC1 | Q9NQX5 | Degrader | |
| RHOBTB1 | O94844 | Degrader | E3 (CRL adaptor) | NPL | Q9BXD5 | Stabilizer | |
| KLHL6 | Q8WZ60 | Degrader | E3 (CRL adaptor) | NPM3 | O75607 | Stabilizer | |
| FBXL8 | Q96CD0 | Stabilizer | E3 (CRL adaptor) | NPSR1 | Q6W5P4 | Degrader | |
| FBXO2 | Q9UK22 | Stabilizer | E3 (CRL adaptor) | NQO1 | P15559 | Stabilizer | |
| CDCA3 | Q99618 | Stabilizer | E3 (CRL adaptor) | NQO2 | P16083 | Stabilizer | |
| SKP1 | P63208 | Stabilizer | E3 (CRL adaptor) | NRSN1 | Q8IZ57 | Degrader | |
| ASB9 | Q96DX5 | Stabilizer | E3 (CRL adaptor) | NRSN2 | Q9GZP1 | Degrader | |
| ELOB | Q15370 | Stabilizer | E3 (CRL adaptor) | NSFL1C | Q9UNZ2 | Stabilizer | |
| ZFP161 | O43829 | Stabilizer | E3 (CRL adaptor) | NT5C1A | Q9BXI3 | Stabilizer | |
| KCTD17 | Q8N5Z5 | Stabilizer | E3 (CRL adaptor) | NT5C3 | Q9HOPO | Degrader | |
| ZBTB18 | Q99592 | Stabilizer | E3 (CRL adaptor) | NUCB1 | Q02818 | Stabilizer | |
| ZBTB7B | O15156 | Stabilizer | E3 (CRL adaptor) | NUDC | Q9Y266 | Stabilizer | |
| KCTD5 | Q9NXV2 | Stabilizer | E3 (CRL adaptor) | NUDT14 | O95848 | Stabilizer | |
| ZBTB20 | Q9HC78 | Stabilizer | E3 (CRL adaptor) | NUDT15 | Q9NV35 | Stabilizer | |
| ZBTB43 | O43298 | Stabilizer | E3 (CRL adaptor) | NUDT16L1 | Q9BRJ7 | Stabilizer | |
| KEAP1 | Q14145 | Stabilizer | E3 (CRL adaptor) | NUDT18 | Q6ZVK8 | Stabilizer | |
| ZBTB10 | Q96DT7 | Stabilizer | E3 (CRL adaptor) | NUDT3 | O95989 | Stabilizer | |
| USP13 | Q92995 | Stabilizer | | NUMB | P49757 | Degrader | |
| USP39 | Q53GS9 | Stabilizer | | NXN | Q6DKJ4 | Stabilizer | |
| USP38 | Q8NB14 | Stabilizer | | OCM | P0CE72 | Stabilizer | |
| USP14 | P54578 | Stabilizer | | OCM2 | P0CE71 | Stabilizer | |
| | | | | OGFR | Q9NZT2 | Stabilizer | |
| | | | | OMP | P47874 | Stabilizer | |
| | | | | OPN1SW | P03999 | Degrader | |
| | | | | OR13A1 | Q8NGR1 | Degrader | |
| | | | | OR14I1 | A6ND48 | Degrader | |
| Gene name | Uniprot | Degrader/ | E3 ligase | OR1J2 | Q8NGS2 | Degrader | |
| | | stabilizer | | | | | |
| A2LD1 | Q9BVM4 | Stabilizer | | OR2H2 | O95918 | Degrader | |
| ABCF3 | Q9NUQ8 | Degrader | | OR51B5 | Q9H339 | Stabilizer | |
| ABCF3 | Q9NUQ8 | Stabilizer | | OR6A2 | O95222 | Degrader | |
| ABHD14A | Q9BUJ0 | Degrader | | OR7D2 | Q96RA2 | Degrader | |
| ABTB1 | Q969K4 | Degrader | E3 (CRL adaptor) | ORAI2 | Q96SN7 | Degrader | |
| ACCS | Q96QU6 | Stabilizer | | OTUD6A | Q7L8S5 | Stabilizer | |
| ACOT1 | Q86TX2 | Stabilizer | | OVOL2 | Q9BRP0 | Degrader | |
| ACOT2 | P49753 | Stabilizer | | P2RY1 | P47900 | Stabilizer | |
| ACOT7 | O00154 | Stabilizer | | PAFAH1B3 | Q15102 | Stabilizer | |
| ACTB | P60709 | Stabilizer | | PAGE1 | O75459 | Stabilizer | |
| ACTC1 | P68032 | Stabilizer | | PAGE2 | Q7Z2X7 | Stabilizer | |
| ACTR1B | P42025 | Stabilizer | | PAGE2B | Q5JRK9 | Stabilizer | |
| ACY3 | Q96HD9 | Stabilizer | | PAGE4 | O60829 | Stabilizer | |
| ADAM17 | P78536 | Degrader | | PAK4 | O96013 | Stabilizer | |
| ADRB2 | P07550 | Stabilizer | | PARM1 | Q6UWI2 | Degrader | |
| ADRB3 | P13945 | Stabilizer | | PCBD1 | P61457 | Stabilizer | |
| AGPAT1 | Q99943 | Stabilizer | | PCBP3 | P57721 | Stabilizer | |
| AGXT2L2 | Q8IUZ5 | Stabilizer | | PCGF1 | Q9BSM1 | Degrader | |
| AHCY | P23526 | Stabilizer | | PCGF2 | P35227 | Stabilizer | |
| AHNAK | Q09666 | Stabilizer | | PCGF3 | Q3KNV8 | Degrader | |
| AHSP | Q9NZD4 | Stabilizer | | PCNP | Q8WW12 | Stabilizer | |
| AKR1B1 | P15121 | Stabilizer | | PCP2 | Q8IVA1 | Stabilizer | |
| AKR1B10 | O60218 | Stabilizer | | PCP4 | P48539 | Stabilizer | |
| AKR7A2 | O43488 | Stabilizer | | PCTP | Q9UKL6 | Stabilizer | |
| AKR7A3 | O95154 | Stabilizer | | PDAP1 | Q13442 | Stabilizer | |
| AKT1 | P31749 | Stabilizer | | PDCL3 | Q9H2J4 | Stabilizer | |
| AKT2 | P31751 | Stabilizer | | PDLIM7 | Q9NR12 | Stabilizer | |
| ALDH1L1 | O75891 | Stabilizer | | PDPN | Q86YL7 | Stabilizer | |
| ALDH3A1 | P30838 | Stabilizer | | PEA15 | Q15121 | Stabilizer | |
| ALDOA | P04075 | Stabilizer | | PEPD | P12955 | Stabilizer | |
| ALDOC | P09972 | Stabilizer | | PEX19 | P40855 | Stabilizer | |
| ALKBH2 | Q6NS38 | Degrader | | PFKFB4 | Q16877 | Stabilizer | |
| ALKBH7 | Q9BT30 | Degrader | | PFN1 | P07737 | Stabilizer | |
| ALOX5AP | P20292 | Stabilizer | | PGAM1 | P18669 | Stabilizer | |
| ALPL | P05186 | Degrader | | PGAM2 | P15259 | Stabilizer | |
| ANXA4 | P09525 | Stabilizer | | PGAM4 | Q8NOY7 | Stabilizer | |
| ANXA5 | P08758 | Stabilizer | | PGAP2 | Q9UHJ9 | Degrader | |
| AP5M1 | Q9H0R1 | Degrader | | PGD | P52209 | Stabilizer | |
| APEX1 | P27695 | Stabilizer | | PGP | A6NDG6 | Stabilizer | |
| APOBEC3G | Q9HC16 | Stabilizer | | PHF20L1 | A8MW92 | Stabilizer | |
| APRT | P07741 | Stabilizer | | PHOSPHO1 | Q8TCT1 | Stabilizer | |
| APTX | Q7Z2E3 | Degrader | | PHPT1 | Q9NRX4 | Stabilizer | |
| AQP1 | P29972 | Degrader | | PHYHD1 | Q5SRE7 | Stabilizer | |
| ARF1 | P84077 | Stabilizer | | PI15 | O43692 | Degrader | |
| ARF3 | P61204 | Stabilizer | | PID1 | Q7Z2X4 | Stabilizer | |
| ARF5 | P84085 | Stabilizer | | PINX1 | Q96BK5 | Stabilizer | |
| ARF6 | P62330 | Stabilizer | | PIP4K2C | Q8TBX8 | Stabilizer | |
| ARHGAP8 | P85298 | Stabilizer | | PJA1 | Q8NG27 | Degrader | RING-E3 |
| ARL3 | P36405 | Stabilizer | | PLA2G16 | P53816 | Degrader | |
| ARL8A | Q96BM9 | Stabilizer | | PLIN3 | O60664 | Stabilizer | |
| ARRB1 | P49407 | Stabilizer | | PLP2 | Q04941 | Stabilizer | |
| ASAP3 | Q8TDY4 | Stabilizer | | PM20D2 | Q8IYS1 | Stabilizer | |
| ASB8 | Q9H765 | Degrader | E3 (CRL adaptor) | PMP22 | Q01453 | Stabilizer | |
| ASCL1 | P50553 | Degrader | | PNPLA4 | P41247 | Degrader | |
| ASGR1 | P07306 | Stabilizer | | POLE3 | Q9NRF9 | Stabilizer | |
| ASMTL | O95671 | Stabilizer | | POLR1C | O15160 | Stabilizer | |
| ASPRV1 | Q53RT3 | Stabilizer | | POLR3H | Q9Y535 | Stabilizer | |
| ASPSCR1 | Q9BZE9 | Stabilizer | | POTEH | Q6S545 | Degrader | |
| ASS1 | P00966 | Stabilizer | | PPIL1 | Q9Y3C6 | Stabilizer | |
| ATCAY | Q86WG3 | Stabilizer | | PPIL2 | Q13356 | Degrader | |
| ATG4B | Q9Y4P1 | Stabilizer | | PPIL2 | Q13356 | Stabilizer | |
| ATP1A4 | Q13733 | Degrader | | PPM1F | P49593 | Stabilizer | |
| ATP2A1 | O14983 | Stabilizer | | PPP1R14B | Q96C90 | Stabilizer | |
| ATP2A3 | Q93084 | Stabilizer | | PPP1R14C | Q8TAE6 | Stabilizer | |
| BABAM1 | Q9NWV8 | Stabilizer | | PPP2R1A | P30153 | Stabilizer | |
| BASP1 | P80723 | Stabilizer | | PPP2R4 | Q15257 | Stabilizer | |
| BCAM | P50895 | Stabilizer | | PREB | Q9HCU5 | Degrader | E3 (CRL adaptor) |
| BCAP31 | P51572 | Stabilizer | | PRELP | P51888 | Degrader | |
| BCL2L15 | Q5TBC7 | Stabilizer | | PRKCZ | Q05513 | Stabilizer | |
| BDKRB1 | P46663 | Degrader | | PRKG1 | Q13976 | Stabilizer | |
| BHLHA15 | Q7RTS1 | Stabilizer | | PRMT8 | Q9NR22 | Stabilizer | |
| BLVRA | P53004 | Stabilizer | | PRPH2 | P23942 | Degrader | |
| BPGM | P07738 | Stabilizer | | PRR23B | Q6ZRT6 | Degrader | |
| BSCL2 | Q96G97 | Stabilizer | | PSCA | O43653 | Degrader | |
| BST2 | Q10589 | Degrader | | PSMC3 | P17980 | Stabilizer | |
| BTBD2 | Q9BX70 | Degrader | | PSMD10 | O75832 | Stabilizer | |
| BTN2A3P | Q96KV6 | Stabilizer | | PSMD9 | O00233 | Stabilizer | |
| C10orf122 | Q5VZQ5 | Degrader | | PTGER4 | P35408 | Stabilizer | |
| C10orf35 | Q96D05 | Stabilizer | | PTGIS | Q16647 | Stabilizer | |
| C12orf5 | Q9NQ88 | Stabilizer | | PTMA | P06454 | Stabilizer | |
| C12orf57 | Q99622 | Stabilizer | | PTN | P21246 | Degrader | |
| C14orf159 | Q7Z3D6 | Degrader | | PURB | Q96QR8 | Stabilizer | |
| C16orf45 | Q96MC5 | Stabilizer | | PVALB | P20472 | Stabilizer | |
| C16orf53 | Q9BTK6 | Stabilizer | | PYGL | P06737 | Stabilizer | |
| C16orf7 | Q9Y2B5 | Stabilizer | | QDPR | P09417 | Stabilizer | |
| C17orf103 | Q8N6N6 | Degrader | | QPRT | Q15274 | Stabilizer | |
| C17orf49 | Q8IXM2 | Stabilizer | | QSOX1 | O00391 | Degrader | |
| C17orf62 | Q9BQA9 | Stabilizer | | RAB11B | Q15907 | Stabilizer | |
| C19orf25 | Q9UFG5 | Stabilizer | | RAB33B | Q9H082 | Stabilizer | |
| C19orf47 | Q8N9M1 | Stabilizer | | RAB39A | Q14964 | Stabilizer | |
| C1orf115 | Q9H7X2 | Degrader | | RAB39B | Q96DA2 | Stabilizer | |
| C1orf123 | Q9NWV4 | Stabilizer | | RAB3A | P20336 | Stabilizer | |
| C1orf201 | Q5TH74 | Degrader | | RAB40A | Q8WXH6 | Degrader | RING-E3 |
| C1orf38 | Q5TEJ8 | Degrader | | RAB4A | P20338 | Stabilizer | |
| C1orf43 | Q9BWL3 | Degrader | | RAB7A | P51149 | Degrader | |
| C20orf11 | Q9NWU2 | Degrader | E3 (CRL adaptor) | RABEP2 | Q9H5N1 | Stabilizer | |
| C20orf27 | Q9GZN8 | Stabilizer | | RABL2A | Q9UBK7 | Stabilizer | |
| C22orf43 | Q6PGQ1 | Stabilizer | | RABL2B | Q9UNT1 | Stabilizer | |
| C3orf18 | Q9UK00 | Stabilizer | | RAC2 | P15153 | Stabilizer | |
| C4BPB | P20851 | Degrader | | RAP2A | P10114 | Stabilizer | |
| C6orf106 | Q9H6K1 | Stabilizer | | RAPGEF5 | Q92565 | Stabilizer | |
| C6orf108 | O43598 | Stabilizer | | RBBP4 | Q09028 | Degrader | E3 (CRL adaptor) |
| C6orf62 | Q9GZU0 | Stabilizer | | RBM3 | P98179 | Stabilizer | |
| C7orf41 | Q8N3F0 | Stabilizer | | RBM38 | Q9H0Z9 | Degrader | |
| C9orf142 | Q9BUH6 | Stabilizer | | RBP1 | O95153 | Stabilizer | |
| CA3 | P07451 | Stabilizer | | RBP2 | O15034 | Stabilizer | |
| CA4 | P22748 | Degrader | | RBP7 | Q96R05 | Stabilizer | |
| CALHM1 | Q8IU99 | Degrader | | RCHY1 | Q96PM5 | Degrader | RING-E3 |
| CALHM3 | Q86XJ0 | Degrader | | RCVRN | P35243 | Stabilizer | |
| CALM1 | P0DP23 | Stabilizer | | REEP5 | Q00765 | Stabilizer | |
| CALM2 | P0DP24 | Stabilizer | | REEP6 | Q96HR9 | Stabilizer | |
| CALM3 | P0DP25 | Stabilizer | | RER1 | O15258 | Degrader | |
| CALML3 | P27482 | Stabilizer | | RFWD3 | Q6PCD5 | Degrader | RING-E3 |
| CALML5 | Q9NZT1 | Stabilizer | | RG9MTD3 | Q6PF06 | Stabilizer | |
| CAMK1D | Q8IU85 | Stabilizer | | RGMB | Q6NW40 | Degrader | |
| CAMK4 | Q16566 | Stabilizer | | RHBDD1 | Q8TEB9 | Degrader | |
| CAPS | Q13938 | Stabilizer | | RHBDL1 | O75783 | Degrader | |
| CAPZA1 | P52907 | Degrader | | RHOBTB3 | O94955 | Degrader | E3 (CRL adaptor) |
| CASP14 | P31944 | Stabilizer | | RHOC | P08134 | Stabilizer | |
| CASP7 | P55210 | Stabilizer | | RHOD | O00212 | Degrader | |
| CAT | P04040 | Stabilizer | | RIC8A | Q9NPQ8 | Stabilizer | |
| CBFA2T2 | O43439 | Degrader | | RLBP1 | P12271 | Stabilizer | |
| CBFB | Q13951 | Stabilizer | | RNASEH1 | O60930 | Degrader | |
| CCDC120 | Q96HB5 | Degrader | | RNF111 | Q6ZNA4 | Degrader | RING-E3 |
| CCDC122 | Q5T0U0 | Degrader | | RNF113A | O15541 | Stabilizer | |
| CCDC155 | Q8N6L0 | Degrader | | RNF115 | Q9Y4L5 | Degrader | RING-E3 |
| CCDC43 | Q96MW1 | Stabilizer | | RNF121 | Q9H920 | Degrader | RING-E3 |
| CCDC75 | Q8N954 | Stabilizer | | RNF125 | Q96EQ8 | Degrader | RING-E3 |
| CCDC78 | A2IDD5 | Stabilizer | | RNF138 | Q8WVD3 | Degrader | RING-E3 |
| CCDC88C | Q9P219 | Degrader | | RNF144A | P50876 | Degrader | RING-E3 |
| CCNDBP1 | O95273 | Degrader | | RNF144B | Q7Z419 | Degrader | RING-E3 |
| CD151 | P48509 | Stabilizer | | RNF150 | Q9ULK6 | Degrader | RING-E3 |
| CD200 | P41217 | Stabilizer | | RNF182 | Q8N6D2 | Degrader | RING-E3 |
| CD200R1L | Q6Q8B3 | Degrader | | RNF183 | Q96D59 | Degrader | RING-E3 |
| CD247 | P20963 | Degrader | | RNF185 | Q96GF1 | Degrader | RING-E3 |
| CD274 | Q9NZQ7 | Degrader | | RNF24 | Q9Y225 | Degrader | RING-E3 |
| CD38 | P28907 | Stabilizer | | RNF8 | O76064 | Degrader | RING-E3 |
| CD83 | Q01151 | Degrader | | RNH1 | O60930 | Stabilizer | |
| CDC37 | Q16543 | Stabilizer | | RNPC3 | Q96LT9 | Degrader | |
| CDK15 | Q96Q40 | Stabilizer | | RNPEP | Q9H4A4 | Stabilizer | |
| CDK2 | P24941 | Stabilizer | | RNPS1 | Q15287 | Stabilizer | |
| CDR2L | Q86X02 | Stabilizer | | ROM1 | Q03395 | Degrader | |
| CEP85 | Q6P2H3 | Stabilizer | | RP2 | O75695 | Stabilizer | |
| CERS4 | Q9HA82 | Degrader | | RPIA | P49247 | Stabilizer | |
| CERS6 | Q6ZMG9 | Stabilizer | | RPL17 | P18621 | Stabilizer | |
| CFL1 | P23528 | Stabilizer | | RPL19 | P84098 | Stabilizer | |
| CKB | P12277 | Stabilizer | | RPL29 | P47914 | Stabilizer | |
| CLIC1 | O00299 | Stabilizer | | RPP14 | O95059 | Degrader | |
| CLIC3 | O95833 | Stabilizer | | RPS2 | P15880 | Stabilizer | |
| CLIC5 | Q9NZA1 | Stabilizer | | RPS9 | P46781 | Stabilizer | |
| CLPB | Q9H078 | Degrader | | RPUSD3 | Q6P087 | Degrader | |
| CLVS2 | Q5SYC1 | Stabilizer | | RRAD | P55042 | Degrader | |
| CLYBL | Q8N0X4 | Degrader | | RRAGB | Q5VZM2 | Stabilizer | |
| CMTM2 | Q8TAZ6 | Degrader | | RRP9 | O43818 | Stabilizer | |
| CMTM5 | Q96DZ9 | Stabilizer | | RSU1 | Q15404 | Stabilizer | |
| CMTM8 | Q8IZV2 | Stabilizer | | RTN3 | O95197 | Stabilizer | |
| CNDP2 | Q96KP4 | Stabilizer | | RTN4 | Q9NQC3 | Stabilizer | |
| CNIH2 | Q6PI25 | Degrader | | S100A1 | P23297 | Stabilizer | |
| CNN2 | Q99439 | Stabilizer | | S100A11 | P31949 | Stabilizer | |
| CNN3 | Q15417 | Stabilizer | | S100A13 | Q99584 | Stabilizer | |
| CNP | P09543 | Stabilizer | | S100A3 | P33764 | Stabilizer | |
| COASY | Q13057 | Stabilizer | | S100A4 | P26447 | Stabilizer | |
| COMT | P21964 | Stabilizer | | S100A7 | P31151 | Stabilizer | |
| COPE | O14579 | Stabilizer | | S100A7A | Q86SG5 | Stabilizer | |
| CORO7 | P57737 | Stabilizer | | SAMD7 | Q7Z3H4 | Stabilizer | |
| CPLX3 | Q8WVH0 | Stabilizer | | SAMD8 | Q96LT4 | Degrader | |
| CPLX4 | Q7Z7G2 | Stabilizer | | SAP30 | O75446 | Degrader | |
| CPPED1 | Q9BRF8 | Stabilizer | | SARS | P49591 | Stabilizer | |
| CRABP1 | P29762 | Stabilizer | | SAT2 | Q96F10 | Stabilizer | |
| CRB3 | Q9BUF7 | Stabilizer | | SC5DL | O75845 | Degrader | |
| CREB3 | O43889 | Degrader | | SCAMP4 | Q969E2 | Stabilizer | |
| CREB3L1 | Q96BA8 | Degrader | | SCGB2A2 | Q13296 | Degrader | |
| CRIP2 | P52943 | Stabilizer | | SCGN | O76038 | Stabilizer | |
| CRKL | P46109 | Stabilizer | | SCLY | Q96I15 | Stabilizer | |
| CRTC1 | Q6UUV9 | Degrader | | SCRN2 | Q96FV2 | Stabilizer | |
| CRTC1 | Q6UUV9 | Stabilizer | | SCTR | P47872 | Stabilizer | |
| CRYBA1 | P05813 | Stabilizer | | SCUBE1 | Q8IWY4 | Stabilizer | |
| CRYGB | P07316 | Stabilizer | | SDHB | P21912 | Degrader | |
| CRYGC | P07315 | Stabilizer | | SEC13 | P55735 | Stabilizer | |
| CRYGD | P07320 | Stabilizer | | SEC14L2 | O76054 | Stabilizer | |
| CRYGS | P22914 | Stabilizer | | SEC14L3 | Q9UDX4 | Stabilizer | |
| CRYM | Q14894 | Stabilizer | | SERPINB3 | P29508 | Degrader | |
| CTLA4 | P16410 | Degrader | | SERPINB9 | P50453 | Stabilizer | |
| CTSC | P53634 | Degrader | | SERPINF2 | P08697 | Degrader | |
| CXCR7 | P25106 | Degrader | | SFN | P31947 | Stabilizer | |
| CYB5R3 | P00387 | Stabilizer | | SGCA | Q16586 | Degrader | |
| CYGB | Q8WWM9 | Stabilizer | | SGTA | O43765 | Stabilizer | |
| CYLC2 | Q14093 | Degrader | | SH3BGRL2 | Q9UJC5 | Stabilizer | |
| CYYR1 | Q96J86 | Degrader | | SH3BGRL3 | Q9H299 | Stabilizer | |
| DBNL | Q9UJU6 | Stabilizer | | SH3GL2 | Q99962 | Stabilizer | |
| DCAF10 | Q5QP82 | Degrader | E3 (CRL adaptor) | SH3GLB2 | Q9NR46 | Stabilizer | |
| DCAF12 | Q5T6F0 | Degrader | E3 (CRL adaptor) | SH3RF2 | Q8TEC5 | Degrader | RING-E3 |
| DCAF19 | Q9NSI6 | Degrader | E3 (CRL adaptor) | SHD | Q96IW2 | Stabilizer | |
| DCAF2 | Q9NZJ0 | Degrader | E3 (CRL adaptor) | SIX3 | O95343 | Degrader | |
| DCAF3 | Q9C0C7 | Degrader | E3 (CRL adaptor) | SKAP1 | Q86WV1 | Stabilizer | |
| DCAF5 | Q96JK2 | Degrader | E3 (CRL adaptor) | SKP2 | Q13309 | Degrader | E3 (CRL adaptor) |
| DCAF6 | Q58WW2 | Degrader | E3 (CRL adaptor) | SLAIN1 | Q8ND83 | Degrader | |
| DCAF7 | P61962 | Degrader | E3 (CRL adaptor) | SLC1A7 | O00341 | Degrader | |
| DCAF9 | Q8N5D0 | Degrader | E3 (CRL adaptor) | SLC22A9 | Q8IVM8 | Degrader | |
| DCPS | Q96C86 | Stabilizer | | SLC25A10 | Q9UBX3 | Degrader | |
| DCTPP1 | Q9H773 | Stabilizer | | SLC25A30 | Q5SVS4 | Degrader | |
| DDB2 | Q92466 | Degrader | E3 (CRL adaptor) | SLC25A41 | Q8N5S1 | Degrader | |
| DDRGK1 | Q96HY6 | Stabilizer | | SLC25A6 | P12236 | Degrader | |
| DDT | P30046 | Stabilizer | | SLC26A6 | Q9BXS9 | Stabilizer | |
| DDX39B | Q13838 | Stabilizer | | SLC2A8 | Q9NY64 | Stabilizer | |
| DECR2 | Q9NUI1 | Degrader | | SLC37A1 | P57057 | Degrader | |
| DEDD | O75618 | Degrader | | SLC43A2 | Q8N370 | Degrader | |
| DEFB115 | Q30KQ5 | Degrader | | SLC5A2 | P31639 | Degrader | |
| DERL1 | Q9BUN8 | Stabilizer | | SLC9A3R1 | O14745 | Stabilizer | |
| DERL2 | Q9GZP9 | Stabilizer | | SLC9A6 | Q92581 | Stabilizer | |
| DHRSX | Q8N5I4 | Stabilizer | | SNAI2 | O43623 | Stabilizer | |
| DLK1 | P80370 | Degrader | | SNCA | P37840 | Stabilizer | |
| DLL3 | Q9NYJ7 | Degrader | | SNCB | Q16143 | Stabilizer | |
| DNAJB3 | Q8WWWVF6 | Stabilizer | | SNCG | O76070 | Stabilizer | |
| DNAJB9 | Q9UBS3 | Degrader | | SNX12 | Q9UMY4 | Stabilizer | |
| DNAJC22 | Q8N4W6 | Degrader | | SOCS2 | O14508 | Degrader | E3 (CRL adaptor) |
| DNPEP | Q9ULA0 | Stabilizer | | SOD1 | P00441 | Stabilizer | |
| DOHH | Q9BU89 | Stabilizer | | SPANXN4 | Q5MJ08 | Stabilizer | |
| DOK2 | O60496 | Stabilizer | | SPDYE1 | Q8NFV5 | Degrader | |
| DPP3 | Q9NY33 | Stabilizer | | SPG7 | Q9UQ90 | Degrader | |
| DPPA3 | Q6W0C5 | Degrader | | SPHK1 | Q9NYA1 | Stabilizer | |
| DRD1 | P21728 | Stabilizer | | SPPL2B | Q8TCT7 | Degrader | |
| DTD1 | Q8TEA8 | Stabilizer | | SPRY2 | O43597 | Stabilizer | |
| DUPD1 | Q68J44 | Stabilizer | | SPSB1 | Q96BD6 | Degrader | E3 (CRL adaptor) |
| DUS1L | Q6P1R4 | Degrader | | SPSB2 | Q99619 | Degrader | E3 (CRL adaptor) |
| DUSP3 | P51452 | Stabilizer | | SPSB4 | Q96A44 | Degrader | E3 (CRL adaptor) |
| DUSP5 | Q16690 | Degrader | | SRA1 | Q9H7N4 | Stabilizer | |
| DYNLT1 | P63172 | Stabilizer | | SRC | P12931 | Stabilizer | |
| DZIP3 | Q86Y13 | Degrader | RING-E3 | SRD5A1 | P18405 | Degrader | |
| EBLN2 | Q6P2I7 | Degrader | | SRM | P19623 | Stabilizer | |
| EDF1 | O60869 | Stabilizer | | SRSF8 | Q9BRL6 | Stabilizer | |
| EED | O75530 | Degrader | E3 (CRL adaptor) | SRSF9 | Q13242 | Stabilizer | |
| EEF1A2 | Q05639 | Stabilizer | | SS18L2 | Q9UHA2 | Degrader | |
| EEF1B2 | P24534 | Stabilizer | | SSNA1 | O43805 | Stabilizer | |
| EGFL8 | Q99944 | Degrader | | SSTR4 | P31391 | Degrader | |
| EIF2B1 | Q14232 | Stabilizer | | ST3GAL3 | Q11203 | Degrader | |
| EIF2B2 | P49770 | Stabilizer | | ST6GAL1 | P15907 | Degrader | |
| EIF4A1 | P60842 | Stabilizer | | ST6GALNAC3 | Q8NDV1 | Stabilizer | |
| EIF5A | P63241 | Stabilizer | | STAT5A | P42229 | Stabilizer | |
| EIF5A2 | Q9GZV4 | Stabilizer | | STMN1 | P16949 | Stabilizer | |
| ELF5 | Q9UKW6 | Stabilizer | | STXBP3 | 000186 | Degrader | |
| EML1 | O00423 | Degrader | E3 (CRL adaptor) | SULT1A1 | P50225 | Stabilizer | |
| ENDOV | Q8N8Q3 | Degrader | | SULT1A2 | P50226 | Stabilizer | |
| ENO2 | P09104 | Stabilizer | | SULT1A3 | P0DMM9 | Stabilizer | |
| ENOPH1 | Q9UHY7 | Stabilizer | | SULT4A1 | Q9BR01 | Stabilizer | |
| ENOSF1 | Q7L5Y1 | Stabilizer | | SUN5 | Q8TC36 | Degrader | |
| ENPP7 | Q6UWV6 | Degrader | | SURF6 | O75683 | Stabilizer | |
| ENSA | O43768 | Stabilizer | | SYNJ2BP | P57105 | Degrader | |
| EPHX1 | P07099 | Stabilizer | | TAB1 | Q15750 | Stabilizer | |
| EPM2AIP1 | Q7L775 | Stabilizer | | TAF5L | O75529 | Degrader | |
| ETHE1 | O95571 | Stabilizer | | TAGLN | Q01995 | Stabilizer | |
| FABP1 | P07148 | Stabilizer | | TANK | Q92844 | Stabilizer | |
| FABP3 | P05413 | Stabilizer | | TBCA | O75347 | Stabilizer | |
| FABP6 | P51161 | Stabilizer | | TBCB | Q99426 | Stabilizer | |
| FADS3 | Q9Y5Q0 | Stabilizer | | TBCC | Q15814 | Stabilizer | |
| FAM104B | Q5XKR9 | Degrader | | TCF15 | Q12870 | Degrader | |
| FAM107B | Q9H098 | Stabilizer | | TCF21 | O43680 | Degrader | |
| FAM189A2 | Q15884 | Degrader | | TDP1 | Q9NUW8 | Degrader | |
| FAM213B | Q8TBF2 | Stabilizer | | TGM3 | Q08188 | Stabilizer | |
| FAM26E | Q8N5C1 | Degrader | | THG1L | Q9NWX6 | Stabilizer | |
| FAM32A | Q9Y421 | Stabilizer | | THTPA | Q9BU02 | Stabilizer | |
| FAM73B | Q7L4E1 | Degrader | | THY1 | P04216 | Degrader | |
| FAM84B | Q96KN1 | Stabilizer | | TK1 | P04183 | Stabilizer | |
| FBP1 | P09467 | Stabilizer | | TLCD1 | Q96CP7 | Degrader | |
| FBP2 | O00757 | Stabilizer | | TLE2 | Q04725 | Degrader | E3 (CRL adaptor) |
| FBXL2 | Q9UKC9 | Degrader | E3 (CRL adaptor) | TLE3 | Q04726 | Degrader | E3 (CRL adaptor) |
| FBXO10 | Q9UK96 | Degrader | E3 (CRL adaptor) | TMC4 | Q7Z404 | Degrader | |
| FBXO11 | Q86XK2 | Degrader | E3 (CRL adaptor) | TMEM121 | Q9BTD3 | Degrader | |
| FBXO24 | O75426 | Degrader | E3 (CRL adaptor) | TMEM130 | Q8N3G9 | Degrader | |
| FBXW7 | Q969H0 | Degrader | E3 (CRL adaptor) | TMEM141 | Q96I45 | Degrader | |
| FBXW9 | Q5XUX1 | Degrader | E3 (CRL adaptor) | TMEM171 | Q8WVE6 | Degrader | |
| FCGR3A | P08637 | Degrader | | TMEM185A | Q8NFB2 | Stabilizer | |
| FCRLA | Q7L513 | Degrader | | TMEM38B | Q9NVV0 | Degrader | |
| FERMT3 | Q86UX7 | Stabilizer | | TMEM53 | Q6P2H8 | Stabilizer | |
| FGF1 | P05230 | Stabilizer | | TMEM71 | Q6P5X7 | Degrader | |
| FGFR3 | P22607 | Degrader | | TMOD2 | Q9NZR1 | Stabilizer | |
| FHIT | P49789 | Stabilizer | | TMPRSS2 | O15393 | Degrader | |
| FKBP4 | Q02790 | Stabilizer | | TMSB10 | P63313 | Stabilizer | |
| FKBPL | Q9UIM3 | Degrader | | TMSB4Y | O14604 | Stabilizer | |
| FOLR1 | P15328 | Degrader | | TNFRSF1B | P20333 | Degrader | |
| FOXJ1 | Q92949 | Degrader | | TNFSF4 | P23510 | Degrader | |
| FTL | P02792 | Stabilizer | | TOM1 | O60784 | Stabilizer | |
| G6PD | P11413 | Stabilizer | | TOMM34 | Q15785 | Stabilizer | |
| GADD45A | P24522 | Degrader | | TPD52L1 | Q16890 | Stabilizer | |
| GADD45B | O75293 | Degrader | | TPD52L2 | O43399 | Stabilizer | |
| GAGE12B | A1L429 | Stabilizer | | TPI1 | P60174 | Stabilizer | |
| GAGE12E | A1L429 | Stabilizer | | TPK1 | Q9H3S4 | Stabilizer | |
| GAGE2A | Q6NT46 | Stabilizer | | TPM1 | P09493 | Stabilizer | |
| GAGE2E | Q4V326 | Stabilizer | | TPM4 | P67936 | Stabilizer | |
| GAGE8 | Q9UEU5 | Stabilizer | | TPMT | P51580 | Stabilizer | |
| GALE | Q14376 | Stabilizer | | TPPP | O94811 | Stabilizer | |
| GALM | Q96C23 | Stabilizer | | TPPP2 | P59282 | Stabilizer | |
| GAPDH | P04406 | Stabilizer | | TPPP3 | Q9BW30 | Stabilizer | |
| GCFC2 | P16383 | Stabilizer | | TRAF2 | Q12933 | Stabilizer | RING-E3 |
| GDI1 | P31150 | Stabilizer | | TREML2 | Q5T2D2 | Stabilizer | |
| GDPD2 | Q9HCC8 | Stabilizer | | TRIM13 | O60858 | Degrader | RING-E3 |
| GGA1 | Q9UJY5 | Stabilizer | | TRIM3 | O75382 | Stabilizer | RING-E3 |
| GGCT | O75223 | Stabilizer | | TRIM31 | Q9BZY9 | Degrader | RING-E3 |
| GJA3 | Q9Y6H8 | Degrader | | TRIM49 | P0CI25 | Degrader | RING-E3 |
| GJC3 | Q8NFK1 | Degrader | | TRIM5 | Q9C035 | Degrader | RING-E3 |
| GLO1 | Q04760 | Stabilizer | | TRIM56 | Q9BRZ2 | Degrader | RING-E3 |
| GLOD4 | Q9HC38 | Stabilizer | | TRIM59 | Q8IWR1 | Degrader | RING-E3 |
| GLP2R | O95838 | Degrader | | TRPC4AP | Q8TEL6 | Degrader | RING-E3 |
| GMDS | O60547 | Stabilizer | | TSEN15 | Q8WW01 | Stabilizer | |
| GNPDA1 | P46926 | Stabilizer | | TSNARE1 | Q96NA8 | Degrader | |
| GNRHR | P30968 | Degrader | | TSPAN18 | Q96SJ8 | Degrader | |
| GOLGA4 | Q13439 | Stabilizer | | TTC9C | Q8N5M4 | Stabilizer | |
| GOT2 | P00505 | Stabilizer | | TTYH2 | Q9BSA4 | Degrader | |
| GPC1 | P35052 | Degrader | | TTYH3 | Q9C0H2 | Degrader | |
| GPI | P06744 | Stabilizer | | TUBB2A | Q13885 | Stabilizer | |
| GPN2 | Q9H9Y4 | Degrader | | TUBB2B | Q9BVA1 | Stabilizer | |
| GPN3 | Q9UHW5 | Degrader | | TUBB3 | Q13509 | Stabilizer | |
| GPR3 | P46089 | Stabilizer | | TUBB4A | P04350 | Stabilizer | |
| GPT | P24298 | Stabilizer | | TUBB4B | P68371 | Stabilizer | |
| GRAMD3 | Q96HH9 | Degrader | | TUBB6 | Q9BUF5 | Stabilizer | |
| GRAP2 | O75791 | Stabilizer | | TUBB8 | Q3ZCM7 | Stabilizer | |
| GRB2 | P62993 | Stabilizer | | TWF2 | Q6IBS0 | Stabilizer | |
| GRTP1 | Q5TC63 | Degrader | | TXNDC17 | Q9BRA2 | Stabilizer | |
| GSDMA | Q96QA5 | Stabilizer | | TXNRD2 | Q9NNW7 | Stabilizer | |
| GSN | P06396 | Degrader | | TYMS | P04818 | Degrader | |
| GSTA1 | P08263 | Stabilizer | | UBA5 | Q9GZZ9 | Stabilizer | |
| GSTA2 | P09210 | Stabilizer | | UCK2 | Q9BZX2 | Stabilizer | |
| GSTM3 | P21266 | Stabilizer | | UEVLD | Q8IX04 | Degrader | |
| GSTO1 | P78417 | Stabilizer | | UHRF1 | Q96T88 | Stabilizer | RING-E3 |
| GSTP1 | P09211 | Stabilizer | | UNC93A | Q86WB7 | Stabilizer | |
| GSTT2 | P0CG29 | Stabilizer | | USP46 | P62068 | Degrader | |
| GSTT2B | P0CG30 | Stabilizer | | UTY | O14607 | Degrader | |
| GSTZ1 | O43708 | Stabilizer | | VAT1L | Q9HCJ6 | Stabilizer | |
| GUK1 | Q16774 | Stabilizer | | VCX2 | Q9H322 | Stabilizer | |
| H1FOO | Q8IZA3 | Stabilizer | | VCX3A | Q9NNX9 | Stabilizer | |
| H2AFJ | Q9BTM1 | Stabilizer | | VCY | O14598 | Stabilizer | |
| H2AFZ | P0C0S5 | Stabilizer | | VCY1B | O14598 | Stabilizer | |
| HAAO | P46952 | Stabilizer | | VIM | P08670 | Stabilizer | |
| HAGH | Q16775 | Stabilizer | | VPS26B | Q4G0F5 | Stabilizer | |
| HDGF | P51858 | Stabilizer | | VPS37A | Q8NEZ2 | Degrader | |
| HERPUD1 | Q15011 | Degrader | | VSTM1 | Q6UX27 | Stabilizer | |
| HHAT | Q5VTY9 | Stabilizer | | VTN | P04004 | Degrader | |
| HIGD2A | Q9BW72 | Degrader | | WDFY2 | Q96P53 | Stabilizer | |
| HIP1R | O75146 | Stabilizer | | WDR25 | Q64LD2 | Degrader | E3 (CRL adaptor) |
| HIST1H1C | P16403 | Stabilizer | | WDR45 | Q9Y484 | Stabilizer | |
| HIST1H1D | P16402 | Stabilizer | | WDR53 | Q7Z5U6 | Degrader | E3 (CRL adaptor) |
| HIST1H2AC | Q93077 | Stabilizer | | WDR57 | Q96DI7 | Degrader | E3 (CRL adaptor) |
| HIST1H2AD | P20671 | Stabilizer | | WDR61 | Q9GZS3 | Degrader | E3 (CRL adaptor) |
| HIST1H2AE | P04908 | Stabilizer | | WDR63 | Q8IWG1 | Degrader | E3 (CRL adaptor) |
| HIST1H2AG | P0C0S8 | Stabilizer | | WDR76 | Q9H967 | Degrader | E3 (CRL adaptor) |
| HIST1H2AH | Q96KK5 | Stabilizer | | WFDC8 | Q8IUA0 | Degrader | |
| HIST1H2AI | P0C0S8 | Stabilizer | | WIBG | Q9BRP8 | Stabilizer | |
| HIST1H2AJ | Q99878 | Stabilizer | | WIPI1 | Q5MNZ9 | Stabilizer | |
| HIST1H2AK | P0C0S8 | Stabilizer | | WNT3 | P56703 | Degrader | |
| HIST1H2AL | P0C0S8 | Stabilizer | | XAGE2 | Q96GT9 | Stabilizer | |
| HIST1H2AM | P0C0S8 | Stabilizer | | XPNPEP1 | Q9NQW7 | Stabilizer | |
| HIST1H2BF | P62807 | Stabilizer | | YKT6 | O15498 | Stabilizer | |
| HIST1H2BO | P23527 | Stabilizer | | YRDC | Q86U90 | Stabilizer | |
| HIST2H2AA3 | Q6FI13 | Stabilizer | | YWHAG | P61981 | Stabilizer | |
| HIST2H2AA4 | Q6FI13 | Stabilizer | | YWHAH | Q04917 | Stabilizer | |
| HIST2H2AC | Q16777 | Stabilizer | | YWHAQ | P27348 | Stabilizer | |
| HIST3H2A | Q7L7L0 | Stabilizer | | ZBED1 | O96006 | Stabilizer | |
| HLA-C | P10321 | Stabilizer | | ZBTB38 | Q8NAP3 | Stabilizer | |
| HLA-DPB1 | P04440 | Stabilizer | | ZDHHC22 | Q8N966 | Degrader | |
| HLA-DQB1 | P01920 | Stabilizer | | ZDHHC4 | Q9NPG8 | Stabilizer | |
| HLA-DRB1 | P01911 | Stabilizer | | ZDHHC9 | Q9Y397 | Degrader | |
| HLA-DRB3 | P79483 | Stabilizer | | ZER1 | Q7Z7L7 | Degrader | E3 (CRL adaptor) |
| HLA-DRB4 | P13762 | Stabilizer | | ZIK1 | Q3SY52 | Stabilizer | |
| HLA-DRB5 | Q30154 | Stabilizer | | ZNF3 | P17036 | Degrader | |
| HMGN1 | P05114 | Stabilizer | | ZNF394 | Q53GI3 | Degrader | |
| HMGN4 | O00479 | Stabilizer | | ZNF395 | Q9H8N7 | Degrader | |
| HN1 | Q9UK76 | Stabilizer | | ZNF561 | Q8N587 | Degrader | |
| HN1L | Q96RY5 | Stabilizer | | ZNF645 | Q8N7E2 | Degrader | RING-E3 |
| HNMT | P50135 | Stabilizer | | | | | |
| HNRNPC | P07910 | Stabilizer | | | | | |
| HNRPLL | Q8WVV9 | Degrader | | | | | |
| HOXA11 | P31270 | Degrader | | | | | |
| HOXA7 | P31268 | Degrader | | | | | |
| HPCA | P84074 | Stabilizer | | | | | |
| HPCAL4 | Q9UM19 | Stabilizer | | | | | |
| HPD | P32754 | Stabilizer | | | | | |

TABLE 5
Effector polypeptides identified as degrading
or stabilizing GFP-ABI1 (excluding E3 ligases)

| | UniProt | | | UniProt | |
|---|---|---|---|---|---|
| | Accession | Degrader/ | | Accession | Degrader/ |
| Protein | Number | Stabilizer | Protein | Number | Stabilizer |
| ATG14 | Q6ZNE5 | Stabilizer | IGSF21 | Q96ID5 | Degrader |
| BAG6 | P46379 | Degrader | IL1RN | P18510 | Stabilizer |
| DDI1 | Q8WTU0 | Stabilizer | IL21R | Q9HBE5 | Degrader |
| DNAJC14 | Q6Y2X3 | no effect | IMPDH1 | P20839 | Stabilizer |
| EID1 | Q9Y6B2 | Degrader | IRAK1BP1 | Q5VVH5 | Degrader |
| FCGR3B | O75015 | Degrader | ISLR | O14498 | Degrader |
| FOLR2 | P14207 | Degrader | ISYNA1 | Q9NPH2 | Stabilizer |
| GABARAP | O95166 | Degrader | ITFG3 | Q9H0X4 | Stabilizer |
| GABARAPL2 | P60520 | Degrader | IZUMO1 | Q8IYV9 | Degrader |
| GET4 | Q7L5D6 | Degrader | KBTBD5 | Q2TBA0 | Stabilizer |
| GID8 | Q9NWU2 | Stabilizer | KCNA1 | Q09470 | Stabilizer |
| ISG15 | P05161 | Degrader | KCND1 | Q9NSA2 | Stabilizer |
| LDOC1 | O95751 | Degrader | KCND3 | Q9UK17 | Stabilizer |
| LGALS3BP | Q08380 | Degrader | KCNJ15 | Q99712 | Degrader |
| LY6D | Q14210 | Degrader | KCNJ5 | P48544 | Stabilizer |
| LYPD3 | O95274 | Degrader | KCNJ6 | P48051 | Stabilizer |
| MAP1LC3A | Q9H492 | Degrader | KCNK1 | O00180 | Stabilizer |
| MAP1LC3B | Q9GZQ8 | Degrader | KCNK6 | Q9Y257 | Stabilizer |
| MTCH1 | Q9NZJ7 | Degrader | KCTD12 | Q96CX2 | Stabilizer |
| OTUB1 | Q96FW1 | Stabilizer | KHK | P50053 | Stabilizer |
| PLEKHB2 | Q96CS7 | no effect | KIAA1715 | Q9C0E8 | Stabilizer |
| PRNP | P04156 | Stabilizer | KLF4 | O43474 | Degrader |
| PRPS2 | P11908 | Stabilizer | KRT8 | P05787 | Stabilizer |
| PRR20A | P86496 | Degrader | LAPTM4B | Q86VI4 | Degrader |
| RTL8C | A6ZKI3 | Degrader | LAPTM5 | Q13571 | Degrader |
| TEX19 | Q8NA77 | Degrader | LDHB | P07195 | Stabilizer |
| TMEM204 | Q9BSN7 | Degrader | LGALS1 | P09382 | Stabilizer |
| TMEM59 | Q9BXS4 | no effect | LGALS4 | P56470 | Stabilizer |
| UBE2A | P49459 | Degrader | LIN28A | Q9H9Z2 | Stabilizer |
| UBE2B | P63146 | Degrader | LINGO1 | Q96FE5 | Stabilizer |
| UBE2C | O00762 | Degrader | LIPG | Q9Y5X9 | Degrader |
| UBE2D1 | P51668 | Degrader | LIPH | Q8WWY8 | Degrader |
| UBE2D3 | P61077 | Degrader | LMAN2L | Q9H0V9 | Degrader |
| UBE2D4 | Q9Y2X8 | Degrader | LMNA | P02545 | Stabilizer |
| UBE2DNL | Q8IWF7 | Degrader | LRAT | O95237 | Degrader |
| UBE2E2 | Q96LR5 | no effect | LRRC14B | A6NHZ5 | Degrader |
| UBE2E3 | Q969T4 | no effect | LRRC28 | Q86X40 | Degrader |
| UBE2F | Q969M7 | no effect | LRRC56 | Q8IYG6 | Stabilizer |
| UBE2G1 | P62253 | Stabilizer | LRRC57 | Q8N9N7 | Stabilizer |
| UBE2G2 | P60604 | Degrader | LTC4S | Q16873 | Degrader |
| UBE2H | P62256 | no effect | MACROD1 | Q9BQ69 | Stabilizer |
| UBE2I | P63279 | Degrader/ | MAGEA4 | P43358 | Stabilizer |
| | | Stabilizer | | | |
| | | (2 isoforms) | | | |
| UBE2J2 | Q8N2K1 | Degrader | MAPK12 | P53778 | Stabilizer |
| UBE2K | P61086 | no effect | MAPK13 | O15264 | Stabilizer |
| UBE2L3 | P68036 | Stabilizer | MARCHF10 | Q8NA82 | Stabilizer |
| UBE2L6 | O14933 | Stabilizer | MARCKSL1 | P49006 | Stabilizer |
| UBE2M | P61081 | Stabilizer | MB | P02144 | Stabilizer |
| UBE2N | P61088 | Degrader | MED24 | O75448 | Degrader |
| UBE2O | Q9C0C9 | Stabilizer | METTL21A | Q8WXB1 | Stabilizer |
| UBE2Q1 | Q7Z7E8 | Degrader | METTL2A | Q96IZ6 | Degrader |
| UBE2Q2 | Q8WVN8 | no effect | MGLL | Q99685 | Stabilizer |
| UBE2R2 | Q712K3 | Stabilizer | MIF | P03971 | Stabilizer |
| UBE2S | Q16763 | no effect | MKRN1 | Q9UHC7 | Degrader |
| UBE2T | Q9NPD8 | Degrader | MLXIP | Q9HAP2 | Degrader |
| UBE2U | Q5WVX9 | Degrader | MLYCD | O95822 | Stabilizer |
| UBE2V1 | Q13404 | Stabilizer | MMGT1 | Q8N4V1 | Degrader |
| UBE2V2 | Q15819 | Stabilizer | MMP15 | P51511 | Degrader |
| UBE2W | Q96B02 | Degrader | MND1 | Q9BWT6 | Degrader |
| UBE2Z | Q9H832 | Degrader | MOB3C | Q70IA8 | Stabilizer |
| UBL4A | P11441 | Degrader | MPP1 | Q00013 | Degrader |
| UCHL1 | P09936 | Stabilizer | MRAP | Q8TCY5 | Degrader |
| PRNP Fusion | PRNP (P04156) | Degrader | MRAS | O14807 | Degrader |
| Polypeptide | with the C-terminal | | | | |
| | residues 194-223 | | | | |
| | of FCGR3B | | | | |
| | (O75015-1) | | | | |
| DDI1- | DDI1 (Q8WTU0) | Stabilizer | MRFAP1L1 | Q96HT8 | Degrader |
| Fragment 1 | without RVP | | | | |
| | domain | | | | |
| DDI1 - | DDI1 (Q8WTU0) | Stabilizer | MRGPRF | Q96AM1 | Stabilizer |
| Fragment 2 | RVP domain only | | | | |
| DDI1 - | DDI1 (Q8WTU0) | Stabilizer | MRPS34 | P82930 | Degrader |
| Fragment 3 | without UBL | | | | |
| | domain | | | | |
| DDI1 - | DDI1 (Q8WTU0) | Stabilizer | MRTO4 | Q9UKD2 | Stabilizer |
| Fragment 4 | without HDD | | | | |
| | domain | | | | |
| Mutant PRSP2 | P11908 with | Stabilizer | MS4A4A | Q96JQ5 | Degrader |
| Q133P | Q133P mutation | | | | |
| Mutant PRSP2 | P11908 with | Stabilizer | MTHFS | P49914 | Stabilizer |
| H130A | H130A mutation | | | | |
| USP13 | Q92995 | Stabilizer | MTMR14 | Q8NCE2 | Stabilizer |
| USP39 | Q53GS9 | Stabilizer | MTPN | P58546 | Stabilizer |
| USP38 | Q8NB14 | Stabilizer | MTSS1L | Q765P7 | Stabilizer |
| USP14 | P54578 | Stabilizer | MVD | P53602 | Stabilizer |
| | | | MVK | Q03426 | Stabilizer |
| A2LD1 | Q9BVM4 | Stabilizer | MYD88 | Q99836 | Stabilizer |
| ABCF3 | Q9NUQ8 | Degrader | MYL4 | P12829 | Stabilizer |
| ABCF3 | Q9NUQ8 | Stabilizer | MYL9 | P24844 | Degrader |
| ABHD14A | Q9BUJ0 | Degrader | NAA50 | Q9GZZ1 | Degrader |
| ACCS | Q96QU6 | Stabilizer | NADK | O95544 | Stabilizer |
| ACOT1 | Q86TX2 | Stabilizer | NAP1L5 | Q96NT1 | Stabilizer |
| ACOT2 | P49753 | Stabilizer | NCALD | P61601 | Stabilizer |
| ACOT7 | O00154 | Stabilizer | NCCRP1 | Q6ZVX7 | Stabilizer |
| ACTB | P60709 | Stabilizer | NCF2 | P19878 | Stabilizer |
| ACTC1 | P68032 | Stabilizer | NCK2 | O43639 | Stabilizer |
| ACTR1B | P42025 | Stabilizer | NCS1 | P62166 | Stabilizer |
| ACY3 | Q96HD9 | Stabilizer | NDFIP2 | Q9NV92 | Degrader |
| ADAM17 | P78536 | Degrader | NDRG1 | Q92597 | Stabilizer |
| ADRB2 | P07550 | Stabilizer | NECAP2 | Q9NVZ3 | Stabilizer |
| ADRB3 | P13945 | Stabilizer | NIPAL4 | Q0D2K0 | Degrader |
| AGPAT1 | Q99943 | Stabilizer | NKX2-1 | P43699 | Degrader |
| AGXT2L2 | Q8IUZ5 | Stabilizer | NME1 | P15531 | Stabilizer |
| AHCY | P23526 | Stabilizer | NPDC1 | Q9NQX5 | Degrader |
| AHNAK | Q09666 | Stabilizer | NPL | Q9BXD5 | Stabilizer |
| AHSP | Q9NZD4 | Stabilizer | NPM3 | O75607 | Stabilizer |
| AKR1B1 | P15121 | Stabilizer | NPSR1 | Q6W5P4 | Degrader |
| AKR1B10 | O60218 | Stabilizer | NQO1 | P15559 | Stabilizer |
| AKR7A2 | O43488 | Stabilizer | NQO2 | P16083 | Stabilizer |
| AKR7A3 | O95154 | Stabilizer | NRSN1 | Q8IZ57 | Degrader |
| AKT1 | P31749 | Stabilizer | NRSN2 | Q9GZP1 | Degrader |
| AKT2 | P31751 | Stabilizer | NSFL1C | Q9UNZ2 | Stabilizer |
| ALDH1L1 | O75891 | Stabilizer | NT5C1A | Q9BXI3 | Stabilizer |
| ALDH3A1 | P30838 | Stabilizer | NT5C3 | Q9H0P0 | Degrader |
| ALDOA | P04075 | Stabilizer | NUCB1 | Q02818 | Stabilizer |
| ALDOC | P09972 | Stabilizer | NUDC | Q9Y266 | Stabilizer |
| ALKBH2 | Q6NS38 | Degrader | NUDT14 | O95848 | Stabilizer |
| ALKBH7 | Q9BT30 | Degrader | NUDT15 | Q9NV35 | Stabilizer |
| ALOX5AP | P20292 | Stabilizer | NUDT16L1 | Q9BRJ7 | Stabilizer |
| ALPL | P05186 | Degrader | NUDT18 | Q6ZVK8 | Stabilizer |
| ANXA4 | P09525 | Stabilizer | NUDT3 | O95989 | Stabilizer |
| ANXA5 | P08758 | Stabilizer | NUMB | P49757 | Degrader |
| AP5M1 | Q9H0R1 | Degrader | NXN | Q6DKJ4 | Stabilizer |
| APEX1 | P27695 | Stabilizer | OCM | P0CE72 | Stabilizer |
| APOBEC3G | Q9HC16 | Stabilizer | OCM2 | P0CE71 | Stabilizer |
| APRT | P07741 | Stabilizer | OGFR | Q9NZT2 | Stabilizer |
| APTX | Q7Z2E3 | Degrader | OMP | P47874 | Stabilizer |
| AQP1 | P29972 | Degrader | OPN1SW | P03999 | Degrader |
| ARF1 | P84077 | Stabilizer | OR13A1 | Q8NGR1 | Degrader |
| ARF3 | P61204 | Stabilizer | OR14I1 | A6ND48 | Degrader |
| ARF5 | P84085 | Stabilizer | OR1J2 | Q8NGS2 | Degrader |
| ARF6 | P62330 | Stabilizer | OR2H2 | O95918 | Degrader |
| ARHGAP8 | P85298 | Stabilizer | OR51B5 | Q9H339 | Stabilizer |
| ARL3 | P36405 | Stabilizer | OR6A2 | O95222 | Degrader |
| ARL8A | Q96BM9 | Stabilizer | OR7D2 | Q96RA2 | Degrader |
| ARRB1 | P49407 | Stabilizer | ORAI2 | Q96SN7 | Degrader |
| ASAP3 | Q8TDY4 | Stabilizer | OTUD6A | Q7L8S5 | Stabilizer |
| ASCL1 | P50553 | Degrader | OVOL2 | Q9BRP0 | Degrader |
| ASGR1 | P07306 | Stabilizer | P2RY1 | P47900 | Stabilizer |
| ASMTL | O95671 | Stabilizer | PAFAH1B3 | Q15102 | Stabilizer |
| ASPRV1 | Q53RT3 | Stabilizer | PAGE1 | O75459 | Stabilizer |
| ASPSCR1 | Q9BZE9 | Stabilizer | PAGE2 | Q7Z2X7 | Stabilizer |
| ASS1 | P00966 | Stabilizer | PAGE2B | Q5JRK9 | Stabilizer |
| ATCAY | Q86WG3 | Stabilizer | PAGE4 | O60829 | Stabilizer |
| ATG4B | Q9Y4P1 | Stabilizer | PAK4 | O96013 | Stabilizer |
| ATP1A4 | Q13733 | Degrader | PARM1 | Q6UWI2 | Degrader |
| ATP2A1 | O14983 | Stabilizer | PCBD1 | P61457 | Stabilizer |
| ATP2A3 | Q93084 | Stabilizer | PCBP3 | P57721 | Stabilizer |
| BABAM1 | Q9NWV8 | Stabilizer | PCGF1 | Q9BSM1 | Degrader |
| BASP1 | P80723 | Stabilizer | PCGF2 | P35227 | Stabilizer |
| BCAM | P50895 | Stabilizer | PCGF3 | Q3KNV8 | Degrader |
| BCAP31 | P51572 | Stabilizer | PCNP | Q8WW12 | Stabilizer |
| BCL2L15 | Q5TBC7 | Stabilizer | PCP2 | Q8IVA1 | Stabilizer |
| BDKRB1 | P46663 | Degrader | PCP4 | P48539 | Stabilizer |
| BHLHA15 | Q7RTS1 | Stabilizer | PCTP | Q9UKL6 | Stabilizer |
| BLVRA | P53004 | Stabilizer | PDAP1 | Q13442 | Stabilizer |
| BPGM | P07738 | Stabilizer | PDCL3 | Q9H2J4 | Stabilizer |
| BSCL2 | Q96G97 | Stabilizer | PDLIM7 | Q9NR12 | Stabilizer |
| BST2 | Q10589 | Degrader | PDPN | Q86YL7 | Stabilizer |
| BTBD2 | Q9BX70 | Degrader | PEA15 | Q15121 | Stabilizer |
| BTN2A3P | Q96KV6 | Stabilizer | PEPD | P12955 | Stabilizer |
| C10orf122 | Q5VZQ5 | Degrader | PEX19 | P40855 | Stabilizer |
| C10orf35 | Q96D05 | Stabilizer | PFKFB4 | Q16877 | Stabilizer |
| C12orf5 | Q9NQ88 | Stabilizer | PFN1 | P07737 | Stabilizer |
| C12orf57 | Q99622 | Stabilizer | PGAM1 | P18669 | Stabilizer |
| C14orf159 | Q7Z3D6 | Degrader | PGAM2 | P15259 | Stabilizer |
| C16orf45 | Q96MC5 | Stabilizer | PGAM4 | Q8N0Y7 | Stabilizer |
| C16orf53 | Q9BTK6 | Stabilizer | PGAP2 | Q9UHJ9 | Degrader |
| C16orf7 | Q9Y2B5 | Stabilizer | PGD | P52209 | Stabilizer |
| C17orf103 | Q8N6N6 | Degrader | PGP | A6NDG6 | Stabilizer |
| C17orf49 | Q8IXM2 | Stabilizer | PHF20L1 | A8MW92 | Stabilizer |
| C17orf62 | Q9BQA9 | Stabilizer | PHOSPHO1 | Q8TCT1 | Stabilizer |
| C19orf25 | Q9UFG5 | Stabilizer | PHPT1 | Q9NRX4 | Stabilizer |
| C19orf47 | Q8N9M1 | Stabilizer | PHYHD1 | Q5SRE7 | Stabilizer |
| C1orf115 | Q9H7X2 | Degrader | PI15 | O43692 | Degrader |
| C1orf123 | Q9NWV4 | Stabilizer | PID1 | Q7Z2X4 | Stabilizer |
| C1orf201 | Q5TH74 | Degrader | PINX1 | Q96BK5 | Stabilizer |
| C1orf38 | Q5TEJ8 | Degrader | PIP4K2C | Q8TBX8 | Stabilizer |
| C1orf43 | Q9BWL3 | Degrader | PLA2G16 | P53816 | Degrader |
| C20orf27 | Q9GZN8 | Stabilizer | PLIN3 | O60664 | Stabilizer |
| C22orf43 | Q6PGQ1 | Stabilizer | PLP2 | Q04941 | Stabilizer |
| C3orf18 | Q9UK00 | Stabilizer | PM20D2 | Q8IYS1 | Stabilizer |
| C4BPB | P20851 | Degrader | PMP22 | Q01453 | Stabilizer |
| C6orf106 | Q9H6K1 | Stabilizer | PNPLA4 | P41247 | Degrader |
| C6orf108 | O43598 | Stabilizer | POLE3 | Q9NRF9 | Stabilizer |
| C6orf62 | Q9GZU0 | Stabilizer | POLR1C | O15160 | Stabilizer |
| C7orf41 | Q8N3F0 | Stabilizer | POLR3H | Q9Y535 | Stabilizer |
| C9orf142 | Q9BUH6 | Stabilizer | POTEH | Q6S545 | Degrader |
| CA3 | P07451 | Stabilizer | PPIL1 | Q9Y3C6 | Stabilizer |
| CA4 | P22748 | Degrader | PPIL2 | Q13356 | Degrader |
| CALHM1 | Q8IU99 | Degrader | PPIL2 | Q13356 | Stabilizer |
| CALHM3 | Q86XJ0 | Degrader | PPM1F | P49593 | Stabilizer |
| CALM1 | P0DP23 | Stabilizer | PPP1R14B | Q96C90 | Stabilizer |
| CALM2 | P0DP24 | Stabilizer | PPP1R14C | Q8TAE6 | Stabilizer |
| CALM3 | P0DP25 | Stabilizer | PPP2R1A | P30153 | Stabilizer |
| CALML3 | P27482 | Stabilizer | PPP2R4 | Q15257 | Stabilizer |
| CALML5 | Q9NZT1 | Stabilizer | PRELP | P51888 | Degrader |
| CAMK1D | Q8IU85 | Stabilizer | PRKCZ | Q05513 | Stabilizer |
| CAMK4 | Q16566 | Stabilizer | PRKG1 | Q13976 | Stabilizer |
| CAPS | Q13938 | Stabilizer | PRMT8 | Q9NR22 | Stabilizer |
| CAPZA1 | P52907 | Degrader | PRPH2 | P23942 | Degrader |
| CASP14 | P31944 | Stabilizer | PRR23B | Q6ZRT6 | Degrader |
| CASP7 | P55210 | Stabilizer | PSCA | O43653 | Degrader |
| CAT | P04040 | Stabilizer | PSMC3 | P17980 | Stabilizer |
| CBFA2T2 | O43439 | Degrader | PSMD10 | O75832 | Stabilizer |
| CBFB | Q13951 | Stabilizer | PSMD9 | O00233 | Stabilizer |
| CCDC120 | Q96HB5 | Degrader | PTGER4 | P35408 | Stabilizer |
| CCDC122 | Q5T0U0 | Degrader | PTGIS | Q16647 | Stabilizer |
| CCDC155 | Q8N6L0 | Degrader | PTMA | P06454 | Stabilizer |
| CCDC43 | Q96MW1 | Stabilizer | PTN | P21246 | Degrader |
| CCDC75 | Q8N954 | Stabilizer | PURB | Q96QR8 | Stabilizer |
| CCDC78 | A2IDD5 | Stabilizer | PVALB | P20472 | Stabilizer |
| CCDC88C | Q9P219 | Degrader | PYGL | P06737 | Stabilizer |
| CCNDBP1 | O95273 | Degrader | QDPR | P09417 | Stabilizer |
| CD151 | P48509 | Stabilizer | QPRT | Q15274 | Stabilizer |
| CD200 | P41217 | Stabilizer | QSOX1 | O00391 | Degrader |
| CD200R1L | Q6Q8B3 | Degrader | RAB11B | Q15907 | Stabilizer |
| CD247 | P20963 | Degrader | RAB33B | Q9H082 | Stabilizer |
| CD274 | Q9NZQ7 | Degrader | RAB39A | Q14964 | Stabilizer |
| CD38 | P28907 | Stabilizer | RAB39B | Q96DA2 | Stabilizer |
| CD83 | Q01151 | Degrader | RAB3A | P20336 | Stabilizer |
| CDC37 | Q16543 | Stabilizer | RAB4A | P20338 | Stabilizer |
| CDK15 | Q96Q40 | Stabilizer | RAB7A | P51149 | Degrader |
| CDK2 | P24941 | Stabilizer | RABEP2 | Q9H5N1 | Stabilizer |
| CDR2L | Q86X02 | Stabilizer | RABL2A | Q9UBK7 | Stabilizer |
| CEP85 | Q6P2H3 | Stabilizer | RABL2B | Q9UNT1 | Stabilizer |
| CERS4 | Q9HA82 | Degrader | RAC2 | P15153 | Stabilizer |
| CERS6 | Q6ZMG9 | Stabilizer | RAP2A | P10114 | Stabilizer |
| CFL1 | P23528 | Stabilizer | RAPGEF5 | Q92565 | Stabilizer |
| CKB | P12277 | Stabilizer | RBM3 | P98179 | Stabilizer |
| CLIC1 | O00299 | Stabilizer | RBM38 | Q9H0Z9 | Degrader |
| CLIC3 | O95833 | Stabilizer | RBP1 | O95153 | Stabilizer |
| CLIC5 | Q9NZA1 | Stabilizer | RBP2 | O15034 | Stabilizer |
| CLPB | Q9H078 | Degrader | RBP7 | Q96R05 | Stabilizer |
| CLVS2 | Q5SYC1 | Stabilizer | RCVRN | P35243 | Stabilizer |
| CLYBL | Q8N0X4 | Degrader | REEP5 | Q00765 | Stabilizer |
| CMTM2 | Q8TAZ6 | Degrader | REEP6 | Q96HR9 | Stabilizer |
| CMTM5 | Q96DZ9 | Stabilizer | RER1 | O15258 | Degrader |
| CMTM8 | Q8IZV2 | Stabilizer | RG9MTD3 | Q6PF06 | Stabilizer |
| CNDP2 | Q96KP4 | Stabilizer | RGMB | Q6NW40 | Degrader |
| CNIH2 | Q6PI25 | Degrader | RHBDD1 | Q8TEB9 | Degrader |
| CNN2 | Q99439 | Stabilizer | RHBDL1 | O75783 | Degrader |
| CNN3 | Q15417 | Stabilizer | RHOC | P08134 | Stabilizer |
| CNP | P09543 | Stabilizer | RHOD | O00212 | Degrader |
| COASY | Q13057 | Stabilizer | RIC8A | Q9NPQ8 | Stabilizer |
| COMT | P21964 | Stabilizer | RLBP1 | P12271 | Stabilizer |
| COPE | O14579 | Stabilizer | RNASEH1 | O60930 | Degrader |
| CORO7 | P57737 | Stabilizer | RNF113A | O15541 | Stabilizer |
| CPLX3 | Q8WVH0 | Stabilizer | RNH1 | O60930 | Stabilizer |
| CPLX4 | Q7Z7G2 | Stabilizer | RNPC3 | Q96LT9 | Degrader |
| CPPED1 | Q9BRF8 | Stabilizer | RNPEP | Q9H4A4 | Stabilizer |
| CRABP1 | P29762 | Stabilizer | RNPS1 | Q15287 | Stabilizer |
| CRB3 | Q9BUF7 | Stabilizer | ROM1 | Q03395 | Degrader |
| CREB3 | O43889 | Degrader | RP2 | O75695 | Stabilizer |
| CREB3L1 | Q96BA8 | Degrader | RPIA | P49247 | Stabilizer |
| CRIP2 | P52943 | Stabilizer | RPL17 | P18621 | Stabilizer |
| CRKL | P46109 | Stabilizer | RPL19 | P84098 | Stabilizer |
| CRTC1 | Q6UUV9 | Degrader | RPL29 | P47914 | Stabilizer |
| CRTC1 | Q6UUV9 | Stabilizer | RPP14 | O95059 | Degrader |
| CRYBA1 | P05813 | Stabilizer | RPS2 | P15880 | Stabilizer |
| CRYGB | P07316 | Stabilizer | RPS9 | P46781 | Stabilizer |
| CRYGC | P07315 | Stabilizer | RPUSD3 | Q6P087 | Degrader |
| CRYGD | P07320 | Stabilizer | RRAD | P55042 | Degrader |
| CRYGS | P22914 | Stabilizer | RRAGB | Q5VZM2 | Stabilizer |
| CRYM | Q14894 | Stabilizer | RRP9 | O43818 | Stabilizer |
| CTLA4 | P16410 | Degrader | RSU1 | Q15404 | Stabilizer |
| CTSC | P53634 | Degrader | RTN3 | O95197 | Stabilizer |
| CXCR7 | P25106 | Degrader | RTN4 | Q9NQC3 | Stabilizer |
| CYB5R3 | P00387 | Stabilizer | S100A1 | P23297 | Stabilizer |
| CYGB | Q8WWM9 | Stabilizer | S100A11 | P31949 | Stabilizer |
| CYLC2 | Q14093 | Degrader | S100A13 | Q99584 | Stabilizer |
| CYYR1 | Q96J86 | Degrader | S100A3 | P33764 | Stabilizer |
| DBNL | Q9UJU6 | Stabilizer | S100A4 | P26447 | Stabilizer |
| DCPS | Q96C86 | Stabilizer | S100A7 | P31151 | Stabilizer |
| DCTPP1 | Q9H773 | Stabilizer | S100A7A | Q86SG5 | Stabilizer |
| DDRGK1 | Q96HY6 | Stabilizer | SAMD7 | Q7Z3H4 | Stabilizer |
| DDT | P30046 | Stabilizer | SAMD8 | Q96LT4 | Degrader |
| DDX39B | Q13838 | Stabilizer | SAP30 | O75446 | Degrader |
| DECR2 | Q9NUI1 | Degrader | SARS | P49591 | Stabilizer |
| DEDD | O75618 | Degrader | SAT2 | Q96F10 | Stabilizer |
| DEFB115 | Q30KQ5 | Degrader | SC5DL | O75845 | Degrader |
| DERL1 | Q9BUN8 | Stabilizer | SCAMP4 | Q969E2 | Stabilizer |
| DERL2 | Q9GZP9 | Stabilizer | SCGB2A2 | Q13296 | Degrader |
| DHRSX | Q8N5I4 | Stabilizer | SCGN | O76038 | Stabilizer |
| DLK1 | P80370 | Degrader | SCLY | Q96I15 | Stabilizer |
| DLL3 | Q9NYJ7 | Degrader | SCRN2 | Q96FV2 | Stabilizer |
| DNAJB3 | Q8WWVF6 | Stabilizer | SCTR | P47872 | Stabilizer |
| DNAJB9 | Q9UBS3 | Degrader | SCUBE1 | Q8IWY4 | Stabilizer |
| DNAJC22 | Q8N4W6 | Degrader | SDHB | P21912 | Degrader |
| DNPEP | Q9ULA0 | Stabilizer | SEC13 | P55735 | Stabilizer |
| DOHH | Q9BU89 | Stabilizer | SEC14L2 | O76054 | Stabilizer |
| DOK2 | O60496 | Stabilizer | SEC14L3 | Q9UDX4 | Stabilizer |
| DPP3 | Q9NY33 | Stabilizer | SERPINB3 | P29508 | Degrader |
| DPPA3 | Q6W0C5 | Degrader | SERPINB9 | P50453 | Stabilizer |
| DRD1 | P21728 | Stabilizer | SERPINF2 | P08697 | Degrader |
| DTD1 | Q8TEA8 | Stabilizer | SFN | P31947 | Stabilizer |
| DUPD1 | Q68J44 | Stabilizer | SGCA | Q16586 | Degrader |
| DUS1L | Q6P1R4 | Degrader | SGTA | O43765 | Stabilizer |
| DUSP3 | P51452 | Stabilizer | SH3BGRL2 | Q9UJC5 | Stabilizer |
| DUSP5 | Q16690 | Degrader | SH3BGRL3 | Q9H299 | Stabilizer |
| DYNLT1 | P63172 | Stabilizer | SH3GL2 | Q99962 | Stabilizer |
| EBLN2 | Q6P2I7 | Degrader | SH3GLB2 | Q9NR46 | Stabilizer |
| EDF1 | O60869 | Stabilizer | SHD | Q96IW2 | Stabilizer |
| EEF1A2 | Q05639 | Stabilizer | SIX3 | O95343 | Degrader |
| EEF1B2 | P24534 | Stabilizer | SKAP1 | Q86WV1 | Stabilizer |
| EGFL8 | Q99944 | Degrader | SLAIN1 | Q8ND83 | Degrader |
| EIF2B1 | Q14232 | Stabilizer | SLC1A7 | O00341 | Degrader |
| EIF2B2 | P49770 | Stabilizer | SLC22A9 | Q8IVM8 | Degrader |
| EIF4A1 | P60842 | Stabilizer | SLC25A10 | Q9UBX3 | Degrader |
| EIF5A | P63241 | Stabilizer | SLC25A30 | Q5SVS4 | Degrader |
| EIF5A2 | Q9GZV4 | Stabilizer | SLC25A41 | Q8N5S1 | Degrader |
| ELF5 | Q9UKW6 | Stabilizer | SLC25A6 | P12236 | Degrader |
| ENDOV | Q8N8Q3 | Degrader | SLC26A6 | Q9BXS9 | Stabilizer |
| ENO2 | P09104 | Stabilizer | SLC2A8 | Q9NY64 | Stabilizer |
| ENOPH1 | Q9UHY7 | Stabilizer | SLC37A1 | P57057 | Degrader |
| ENOSF1 | Q7L5Y1 | Stabilizer | SLC43A2 | Q8N370 | Degrader |
| ENPP7 | Q6UWV6 | Degrader | SLC5A2 | P31639 | Degrader |
| ENSA | O43768 | Stabilizer | SLC9A3R1 | O14745 | Stabilizer |
| EPHX1 | P07099 | Stabilizer | SLC9A6 | Q92581 | Stabilizer |
| EPM2AIP1 | Q7L775 | Stabilizer | SNAI2 | O43623 | Stabilizer |
| ETHE1 | O95571 | Stabilizer | SNCA | P37840 | Stabilizer |
| FABP1 | P07148 | Stabilizer | SNCB | Q16143 | Stabilizer |
| FABP3 | P05413 | Stabilizer | SNCG | O76070 | Stabilizer |
| FABP6 | P51161 | Stabilizer | SNX12 | Q9UMY4 | Stabilizer |
| FADS3 | Q9Y5Q0 | Stabilizer | SOD1 | P00441 | Stabilizer |
| FAM104B | Q5XKR9 | Degrader | SPANXN4 | Q5MJ08 | Stabilizer |
| FAM107B | Q9H098 | Stabilizer | SPDYE1 | Q8NFV5 | Degrader |
| FAM189A2 | Q15884 | Degrader | SPG7 | Q9UQ90 | Degrader |
| FAM213B | Q8TBF2 | Stabilizer | SPHK1 | Q9NYA1 | Stabilizer |
| FAM26E | Q8N5C1 | Degrader | SPPL2B | Q8TCT7 | Degrader |
| FAM32A | Q9Y421 | Stabilizer | SPRY2 | O43597 | Stabilizer |
| FAM73B | Q7L4E1 | Degrader | SRA1 | Q9H7N4 | Stabilizer |
| FAM84B | Q96KN1 | Stabilizer | SRC | P12931 | Stabilizer |
| FBP1 | P09467 | Stabilizer | SRD5A1 | P18405 | Degrader |
| FBP2 | O00757 | Stabilizer | SRM | P19623 | Stabilizer |
| FCGR3A | P08637 | Degrader | SRSF8 | Q9BRL6 | Stabilizer |
| FCRLA | Q7L513 | Degrader | SRSF9 | Q13242 | Stabilizer |
| FERMT3 | Q86UX7 | Stabilizer | SS18L2 | Q9UHA2 | Degrader |
| FGF1 | P05230 | Stabilizer | SSNA1 | O43805 | Stabilizer |
| FGFR3 | P22607 | Degrader | SSTR4 | P31391 | Degrader |
| FHIT | P49789 | Stabilizer | ST3GAL3 | Q11203 | Degrader |
| FKBP4 | Q02790 | Stabilizer | ST6GAL1 | P15907 | Degrader |
| FKBPL | Q9UIM3 | Degrader | ST6GALNAC3 | Q8NDV1 | Stabilizer |
| FOLR1 | P15328 | Degrader | STAT5A | P42229 | Stabilizer |
| FOXJ1 | Q92949 | Degrader | STMN1 | P16949 | Stabilizer |
| FTL | P02792 | Stabilizer | STXBP3 | O00186 | Degrader |
| G6PD | P11413 | Stabilizer | SULT1A1 | P50225 | Stabilizer |
| GADD45A | P24522 | Degrader | SULT1A2 | P50226 | Stabilizer |
| GADD45B | O75293 | Degrader | SULT1A3 | P0DMM9 | Stabilizer |
| GAGE12B | A1L429 | Stabilizer | SULT4A1 | Q9BR01 | Stabilizer |
| GAGE12E | A1L429 | Stabilizer | SUN5 | Q8TC36 | Degrader |
| GAGE2A | Q6NT46 | Stabilizer | SURF6 | O75683 | Stabilizer |
| GAGE2E | Q4V326 | Stabilizer | SYNJ2BP | P57105 | Degrader |
| GAGE8 | Q9UEU5 | Stabilizer | TAB1 | Q15750 | Stabilizer |
| GALE | Q14376 | Stabilizer | TAF5L | O75529 | Degrader |
| GALM | Q96C23 | Stabilizer | TAGLN | Q01995 | Stabilizer |
| GAPDH | P04406 | Stabilizer | TANK | Q92844 | Stabilizer |
| GCFC2 | P16383 | Stabilizer | TBCA | O75347 | Stabilizer |
| GDI1 | P31150 | Stabilizer | TBCB | Q99426 | Stabilizer |
| GDPD2 | Q9HCC8 | Stabilizer | TBCC | Q15814 | Stabilizer |
| GGA1 | Q9UJY5 | Stabilizer | TCF15 | Q12870 | Degrader |
| GGCT | O75223 | Stabilizer | TCF21 | O43680 | Degrader |
| GJA3 | Q9Y6H8 | Degrader | TDP1 | Q9NUW8 | Degrader |
| GJC3 | Q8NFK1 | Degrader | TGM3 | Q08188 | Stabilizer |
| GLO1 | Q04760 | Stabilizer | THG1L | Q9NWX6 | Stabilizer |
| GLOD4 | Q9HC38 | Stabilizer | THTPA | Q9BU02 | Stabilizer |
| GLP2R | O95838 | Degrader | THY | P04216 | Degrader |
| GMDS | O60547 | Stabilizer | TK1 | P04183 | Stabilizer |
| GNPDA1 | P46926 | Stabilizer | TLCD1 | Q96CP7 | Degrader |
| GNRHR | P30968 | Degrader | TMC4 | Q7Z404 | Degrader |
| GOLGA4 | Q13439 | Stabilizer | TMEM121 | Q9BTD3 | Degrader |
| GOT2 | P00505 | Stabilizer | TMEM130 | Q8N3G9 | Degrader |
| GPC1 | P35052 | Degrader | TMEM141 | Q96I45 | Degrader |
| GPI | P06744 | Stabilizer | TMEM171 | Q8WVE6 | Degrader |
| GPN2 | Q9H9Y4 | Degrader | TMEM185A | Q8NFB2 | Stabilizer |
| GPN3 | Q9UHW5 | Degrader | TMEM38B | Q9NVV0 | Degrader |
| GPR3 | P46089 | Stabilizer | TMEM53 | Q6P2H8 | Stabilizer |
| GPT | P24298 | Stabilizer | TMEM71 | Q6P5X7 | Degrader |
| GRAMD3 | Q96HH9 | Degrader | TMOD2 | Q9NZR1 | Stabilizer |
| GRAP2 | O75791 | Stabilizer | TMPRSS2 | O15393 | Degrader |
| GRB2 | P62993 | Stabilizer | TMSB10 | P63313 | Stabilizer |
| GRTP1 | Q5TC63 | Degrader | TMSB4Y | O14604 | Stabilizer |
| GSDMA | Q96QA5 | Stabilizer | TNFRSF1B | P20333 | Degrader |
| GSN | P06396 | Degrader | TNFSF4 | P23510 | Degrader |
| GSTA1 | P08263 | Stabilizer | TOM1 | O60784 | Stabilizer |
| GSTA2 | P09210 | Stabilizer | TOMM34 | Q15785 | Stabilizer |
| GSTM3 | P21266 | Stabilizer | TPD52L1 | Q16890 | Stabilizer |
| GSTO1 | P78417 | Stabilizer | TPD52L2 | O43399 | Stabilizer |
| GSTP1 | P09211 | Stabilizer | TPI1 | P60174 | Stabilizer |
| GSTT2 | P0CG29 | Stabilizer | TPK1 | Q9H3S4 | Stabilizer |
| GSTT2B | P0CG30 | Stabilizer | TPM1 | P09493 | Stabilizer |
| GSTZ1 | O43708 | Stabilizer | TPM4 | P67936 | Stabilizer |
| GUK1 | Q16774 | Stabilizer | TPMT | P51580 | Stabilizer |
| H1FOO | Q8IZA3 | Stabilizer | TPPP | O94811 | Stabilizer |
| H2AFJ | Q9BTM1 | Stabilizer | TPPP2 | P59282 | Stabilizer |
| H2AFZ | P0C0S5 | Stabilizer | TPPP3 | Q9BW30 | Stabilizer |
| HAAO | P46952 | Stabilizer | TREML2 | Q5T2D2 | Stabilizer |
| HAGH | Q16775 | Stabilizer | TSEN15 | Q8WW01 | Stabilizer |
| HDGF | P51858 | Stabilizer | TSNARE1 | Q96NA8 | Degrader |
| HERPUD1 | Q15011 | Degrader | TSPAN18 | Q96SJ8 | Degrader |
| HHAT | Q5VTY9 | Stabilizer | TTC9C | Q8N5M4 | Stabilizer |
| HIGD2A | Q9BW72 | Degrader | TTYH2 | Q9BSA4 | Degrader |
| HIP1R | O75146 | Stabilizer | TTYH3 | Q9C0H2 | Degrader |
| HIST1H1C | P16403 | Stabilizer | TUBB2A | Q13885 | Stabilizer |
| HIST1H1D | P16402 | Stabilizer | TUBB2B | Q9BVA1 | Stabilizer |
| HIST1H2AC | Q93077 | Stabilizer | TUBB3 | Q13509 | Stabilizer |
| HIST1H2AD | P20671 | Stabilizer | TUBB4A | P04350 | Stabilizer |
| HIST1H2AE | P04908 | Stabilizer | TUBB4B | P68371 | Stabilizer |
| HIST1H2AG | P0C0S8 | Stabilizer | TUBB6 | Q9BUF5 | Stabilizer |
| HIST1H2AH | Q96KK5 | Stabilizer | TUBB8 | Q3ZCM7 | Stabilizer |
| HIST1H2AI | P0C0S8 | Stabilizer | TWF2 | Q6IBS0 | Stabilizer |
| HIST1H2AJ | Q99878 | Stabilizer | TXNDC17 | Q9BRA2 | Stabilizer |
| HIST1H2AK | P0C0S8 | Stabilizer | TXNRD2 | Q9NNW7 | Stabilizer |
| HIST1H2AL | P0C0S8 | Stabilizer | TYMS | P04818 | Degrader |
| HIST1H2AM | P0C0S8 | Stabilizer | UBA5 | Q9GZZ9 | Stabilizer |
| HIST1H2BF | P62807 | Stabilizer | UCK2 | Q9BZX2 | Stabilizer |
| HIST1H2BO | P23527 | Stabilizer | UEVLD | Q8IX04 | Degrader |
| HIST2H2AA3 | Q6FI13 | Stabilizer | UNC93A | Q86WB7 | Stabilizer |
| HIST2H2AA4 | Q6FI13 | Stabilizer | USP46 | P62068 | Degrader |
| HIST2H2AC | Q16777 | Stabilizer | UTY | O14607 | Degrader |
| HIST3H2A | Q7L7L0 | Stabilizer | VAT1L | Q9HCJ6 | Stabilizer |
| HLA-C | P10321 | Stabilizer | VCX2 | Q9H322 | Stabilizer |
| HLA-DPB1 | P04440 | Stabilizer | VCX3A | Q9NNX9 | Stabilizer |
| HLA-DQB1 | P01920 | Stabilizer | VCY | O14598 | Stabilizer |
| HLA-DRB1 | P01911 | Stabilizer | VCY1B | O14598 | Stabilizer |
| HLA-DRB3 | P79483 | Stabilizer | VIM | P08670 | Stabilizer |
| HLA-DRB4 | P13762 | Stabilizer | VPS26B | Q4G0F5 | Stabilizer |
| HLA-DRB5 | Q30154 | Stabilizer | VPS37A | Q8NEZ2 | Degrader |
| HMGN1 | P05114 | Stabilizer | VSTM1 | Q6UX27 | Stabilizer |
| HMGN4 | O00479 | Stabilizer | VTN | P04004 | Degrader |
| HN1 | Q9UK76 | Stabilizer | WDFY2 | Q96P53 | Stabilizer |
| HN1L | Q96RY5 | Stabilizer | WDR45 | Q9Y484 | Stabilizer |
| HNMT | P50135 | Stabilizer | WFDC8 | Q8IUA0 | Degrader |
| HNRNPC | P07910 | Stabilizer | WIBG | Q9BRP8 | Stabilizer |
| HNRPLL | Q8WVV9 | Degrader | WIPI1 | Q5MNZ9 | Stabilizer |
| HOXA11 | P31270 | Degrader | WNT3 | P56703 | Degrader |
| HOXA7 | P31268 | Degrader | XAGE2 | Q96GT9 | Stabilizer |
| HPCA | P84074 | Stabilizer | XPNPEP1 | Q9NQW7 | Stabilizer |
| HPCAL4 | Q9UM19 | Stabilizer | YKT6 | O15498 | Stabilizer |
| HPD | P32754 | Stabilizer | YRDC | Q86U90 | Stabilizer |
| HPRT1 | P00492 | Stabilizer | YWHAG | P61981 | Stabilizer |
| HSD17B1 | P14061 | Stabilizer | YWHAH | Q04917 | Stabilizer |
| HSD17B14 | Q9BPX1 | Stabilizer | YWHAQ | P27348 | Stabilizer |
| HSPA12A | O43301 | Stabilizer | ZBED1 | O96006 | Stabilizer |
| HSPA1A | P0DMV8 | Stabilizer | ZBTB38 | Q8NAP3 | Stabilizer |
| HSPA1B | P0DMV9 | Stabilizer | ZDHHC22 | Q8N966 | Degrader |
| HTRA3 | P83110 | Degrader | ZDHHC4 | Q9NPG8 | Stabilizer |
| HYAL3 | O43820 | Degrader | ZDHHC9 | Q9Y397 | Degrader |
| ICAM2 | P13598 | Stabilizer | ZIK1 | Q3SY52 | Stabilizer |
| IDH2 | P48735 | Degrader | ZNF3 | P17036 | Degrader |
| IGF2BP2 | Q9Y6M1 | Stabilizer | ZNF394 | Q53GI3 | Degrader |
| IGHM | P01871 | Stabilizer | ZNF395 | Q9H8N7 | Degrader |
| IGSF1 | Q8N6C5 | Degrader | ZNF561 | Q8N587 | Degrader |

### Validation of Screen Hits

Selected hits from the screen were first validated by individually transfecting them as vhhGFP fusions into the original ABI1-GFP cell line and assessing their effect on the reporter stability. Most degradation screen hits robustly degraded the reporter, whereas both of the two tested hits from the stabilization screen (DDI1 and PRPS2) increased the GFP signal (FIG. 2). The effect of several inhibitors of protein homeostasis pathways on effector function were then assessed. Treatment of cells with the proteasome inhibitor bortezomib inhibited most degradation effectors, consistent with them being dependent on proteasome function (FIG. 2). In contrast, inhibition of translation with cycloheximide globally increased the degradative effect of the tested hits (FIG. 2). Next, two compounds targeting more specific aspects of protein degradation were tested, the neddylation inhibitor MLN4924 and the VCP/p97 inhibitor CB-5083 (REFs). MLN4924, which specifically interferes with the function of cullin-RING E3 ligases (CRLs), inhibited several CRL adaptor degraders but had little effect on other degraders (FIG. 2). Interestingly, not all CRLs were equally affected by MLN4924 treatment. For example, FBXL14 was less affected by MLN4924 than related F box proteins FBXL12 and FBXL15. CB-5083 also interfered with the degradation activity of some but not other effectors (FIG. 2). While it did not generally affect the ability of E3 ligases to degrade the reporter, it did inhibit the function of CRBN. CB-5083 inhibited the activity of LC3A, GABARAP and GABARAPL2, three central regulators of autophagy.

### Unexpected Degraders

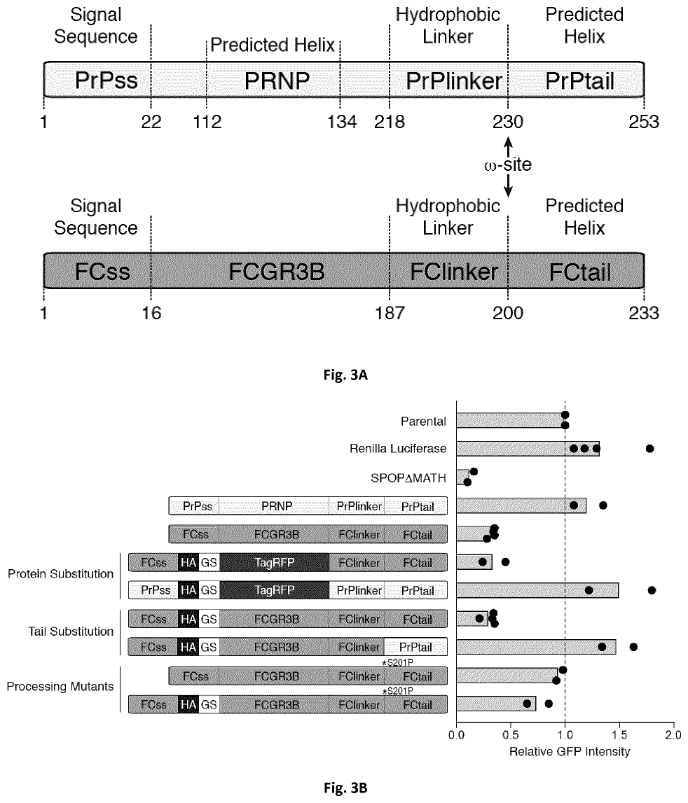

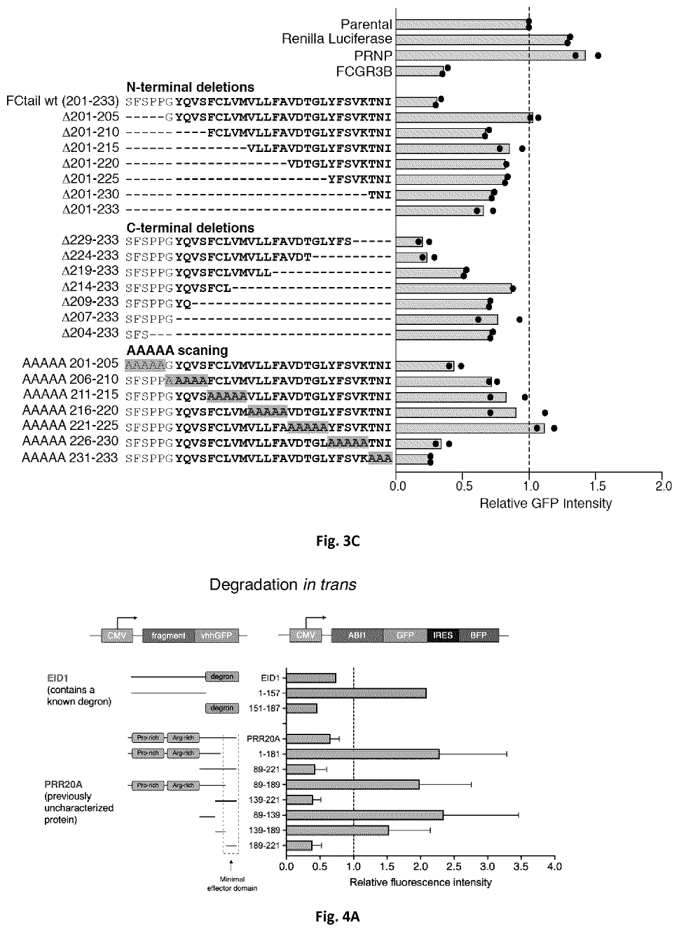

Although the screen hits were highly enriched in E3 ligases, they also included unexpected factors and completely uncharacterized proteins. For example, several GPI anchored proteins were identified and validated as potent degraders, including FCGR3B, LY6D and LYPD3 (FIG. 3A). In contrast, the prion protein (PRNP), a well-characterized GPI anchored protein, was neither a screen hit nor degraded ABI1-GFP when individually tested (FIG. 3A). To identify the molecular determinants of this difference, FCGR3B (also known as CD16b) and PRNP were focused on. Both proteins have a signal peptide followed by a folded domain and a GPI anchor signal that consists of a hydrophilic linker, cleavage site, and a hydrophobic tail (FIG. 3B). The folded domains of PRNP and FCGR3B with were first replaced with TagRFP, while keeping their signal sequences and GPI anchor signals. These constructs behaved identically to the unmodified proteins, suggesting that the folded domains are not involved in GFP-ABI1 degradation (FIG. 3B). In contrast, the C-terminal hydrophobic tails of FCGR3B and PRNP were then swapped, their activities reversed: FCGR3B with the PRNP tail did not degrade the reporter, whereas PRNP with the FCGR3B tail did (FIG. 3B). Thus, the C-terminal hydrophobic tail is responsible for the difference between PRNP and FCGR3B in the degradation assay.

The degradation-conferring region in FCGR3B with deletion constructs were further defined. Deletion of five amino acids immediately following the w cleavage site abolished the activity of FCGR3B (FIG. 3C). The C-terminus of the hydrophobic tail was more tolerant to deletions, as deletion of the last 10 amino acids did not notably affect the activity. However, deletion of amino acids 219-223 or replacing aa 221-225 with alanines significantly reduced the ability of FCGR3B to degrade the reporter, suggesting that the center residues of the hydrophobic tail are functionally important. Finally, it was determined if preventing the cleavage and processing of the C-terminal tail is functionally relevant. It was: mutating Ser201, which is immediately adjacent to the predicted cleavage site, abolished the activity (FIG. 3A). Together, these results indicate that the ability of FCGR3B to degrade the cytoplasmic GFP-ABI1 reporter requires proper GPI anchor processing and the central core of the hydrophobic tail.

### Degradation in Trans Via Degron Motifs

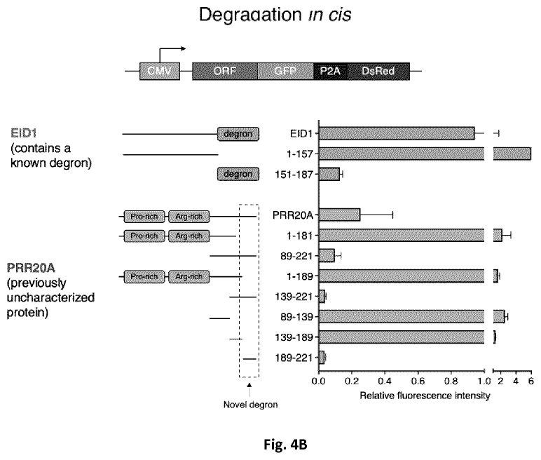

Several uncharacterized proteins were identified as potent degraders in the screen. One of these was PRR20A, an uncharacterized proline-rich protein. PRR20A does not contain any globular domains (as predicted by AlphaFold2), so it was hypothesized that it might function by recruiting an E3 ligase or another factor through a degron sequence. This hypothesis was bolstered by the observation that another vhhGFP screen hit was EID1, a protein that contains a degron that binds the E3 ligase adaptor FBXO21 (Watanabe et al., 2015; Zhang et al., 2015). Degrons can induce degradation of stable proteins in cis when fused to them, whereas “canonical” degraders such as E3 ligases will not do so. It was therefore tested if EID1 and PRR20A (or their fragments) can induce degradation both in trans and in cis. For in trans degradation, vhhGFP fusions targeting the ABI1-GFP reporter (FIG. 4A) were used. For in cis degradation, fused fragments of PRR20A and EID1 to GFP in a vectorthat also expresses DsRed under control of an internal ribosomal entry site (IRES) were used. In this context, fluorescence ratio between the GFP fusion and DsRed control acts as a stability reporter (FIG. 4B). As expected, the fusion of GFP to EID1 C terminus, which contains the FBXO21 degron, led to low GFP fluorescence, whereas EID1 N terminus fused to GFP was stable (FIG. 4B). Full-length PRR20A fused to GFP was also unstable. Using multiple overlapping fragments of PRR20A, the C-terminal residues 189-221 were identified as the region conferring low stability to GFP fusions (FIG. 4B). Notably, this fragment could also degrade GFP-ABI1 when fused to vhhGFP, indicating that the same region harbors both in cis and in trans degradation activity (FIG. 4A). More generally, these results indicate that induced proximity screens can identify degraders that work through multiple mechanisms.

### UBE2B is a Highly Potent Degrader

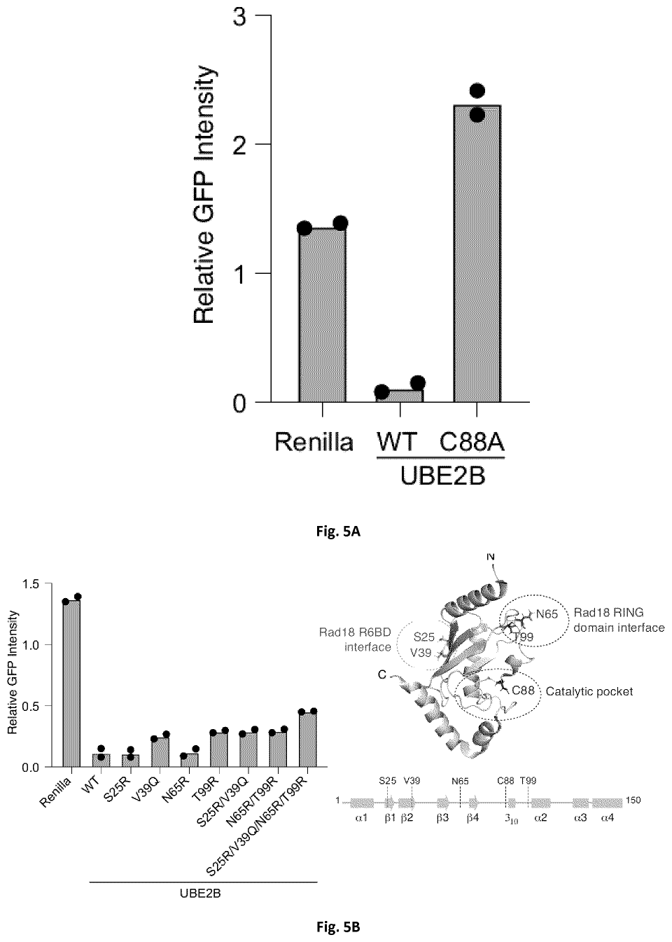

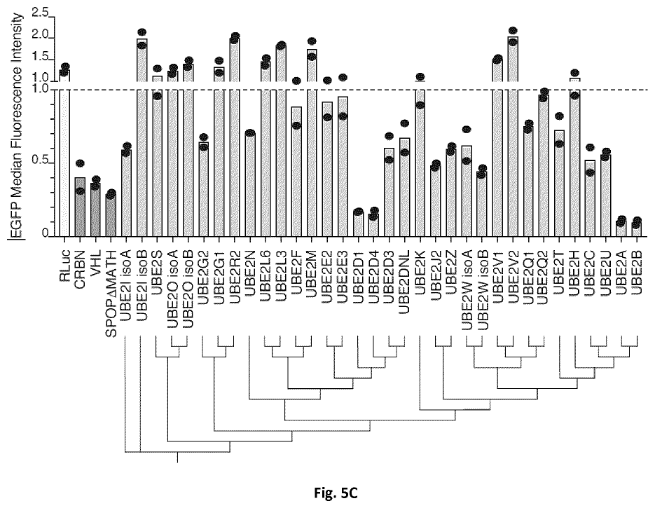

One of the most potent degraders identified was the E2 conjugating enzyme UBE2B. It was a hit in both vhhGFP and PYL1 fusion screens (FIG. 2). UBE2B was a particularly interesting effector, as E2 enzymes have not been previously harnessed in targeted protein degradation. To further characterize UBE2B, it was tested whether its catalytic activity is required for degradation. The active site of UBE2B contains Cys88, which is transiently conjugated to ubiquitin via transthiolation reaction before ubiquitin transfer to the substrate or a HECT-type E3 ligase (Stewart et al., 2016). Mutating Cys88 to alanine (UBE2B C88A) completely abolished the activity, indicating that transthiolation is required for proximity-induced degradation by UBE2B (FIG. 5A).

Because E2s always function in tandem with E3 ligases in target ubiquitination, it was next asked if UBE2B requires an E3 partner to promote proximity-dependent degradation. UBE2B interacts with the E3 ligase RAD18 through two interfaces on opposite sides of the protein. Disruptive mutations in the interface that interacts with RAD18 RING finger (N65R, T99R), the interface that contacts the R6BD domain of RAD18 (S25R, V39Q), or combined all four mutations were introduced. All mutants were still active in the degradation assay, indicating that E3 binding is, surprisingly, not required for the ability of UBE2B to degrade targets in a proximity-dependent manner (FIG. 5B).

To study if E2 conjugating enzymes are generally potent degraders, the activity of 30 of the 38 human E2s were assayed in the degradation assay (FIG. 5C). Only few E2s were highly active in the assay. UBE2B was the most potent degrader together with its paralog UBE2A, which is 95% identical in sequence (FIG. 5C). In addition, related E2s UBE2D1 and UBE2D4 were also highly active in the assay. Notably, UBE2D4 can also ubiquitinate a substrate protein in vitro in an E3-independent manner (David et al., 2010). These results indicate there are inherent functional differences between the E2 family members despite the highly conserved fold. More generally, these results suggest that some (but not all) E2s could be harnessed for targeted protein degradation.

### Identification of Proximity-Dependent Stabilizers

Several stabilizers were also discovered in the screen. For example, the deubiquitinase OTUB1 was identified as a hit in the PYL1/ABI1 proximity screen. OTUB1 has been used as an effector for deubiquitinase-targeting chimeras (DUBTACs)(Henning et al., 2021), indicating that the screen identified relevant proximity-dependent effectors. Prominent hits in the vhhGFP screen were the ubiquitin-dependent protease DDI1 (Yip et al., 2020) and the pyrophosphokinase PRPS2. Moreover, while optimizing the large-scale screen and testing multiple E3 ligase fusions to vhhGFP, it was noticed that the putative ubiquitin ligase KLHL40 did not degrade the reporter but rather stabilized it (FIG. 2A). Therefore, DDI1, PRPS2 and KLHL40 were focused on as putative stabilization effectors.

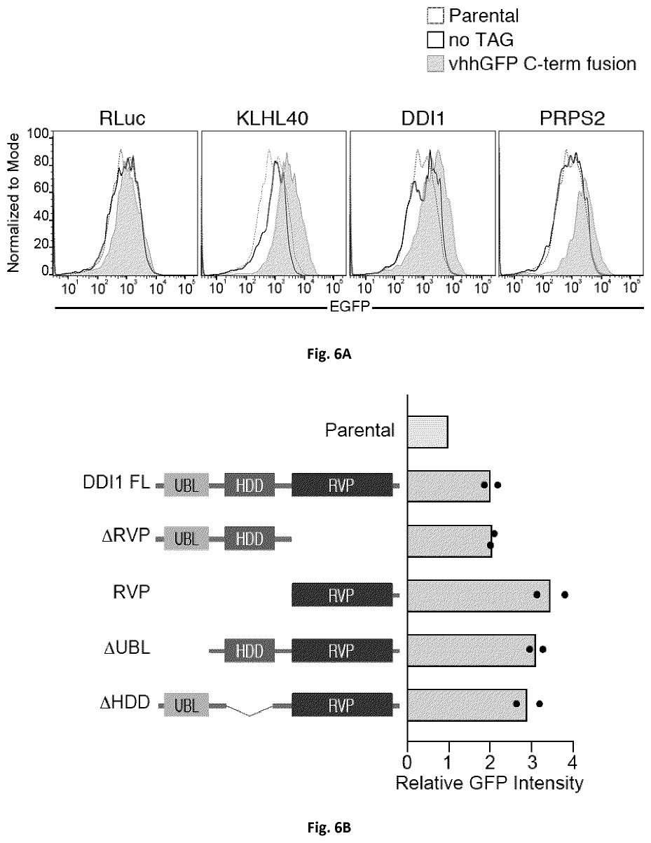

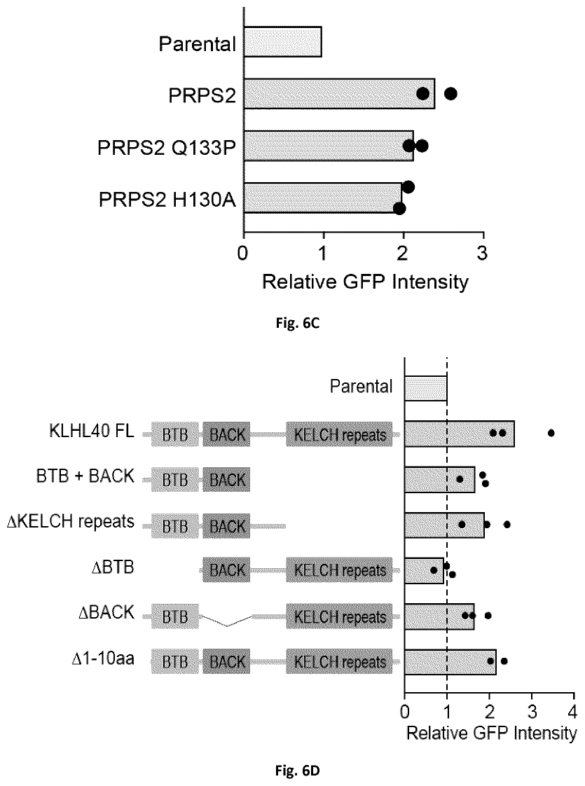

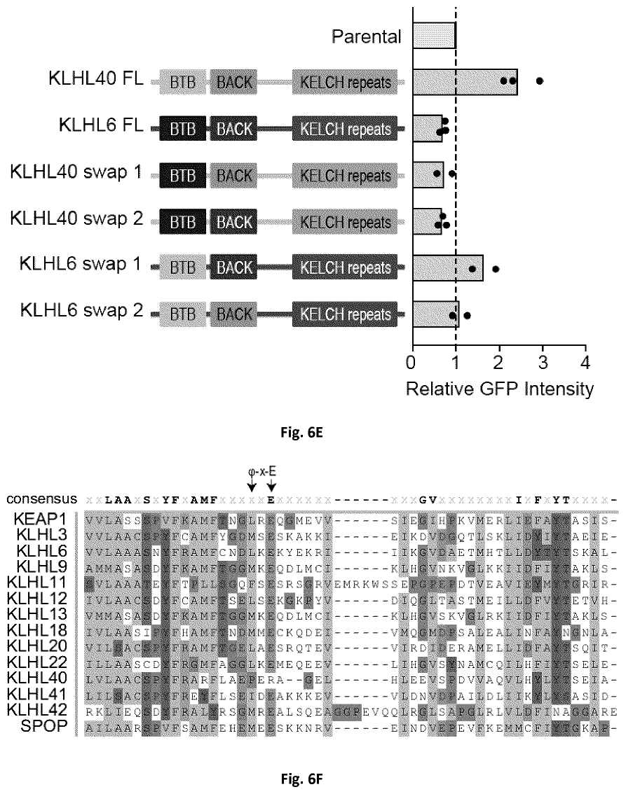

It was first tested if these proteins stabilize the reporter due to indirect effects on protein homeostasis or if they require proximity to the reporter. Removing the vhhGFP tag abolished their activity in the degradation assay, indicating that they do not affect protein degradation nonspecifically (FIG. 6A). To identify regions that are required for the activity of these proteins, deletion and mutant constructs were tested. DDI1 contains an N-terminal ubiquitin-like domain and a C-terminal retrovirus-like aspartate protease domain (FIG. 6B). Surprisingly, multiple fragments of DDI1 could stabilize the reporter, indicating that its activity is not limited to a single region (FIG. 6B). For PRPS2, a construct with mutations in the catalytic site was tested to determine if its enzymatic activity is required for stabilization. It was not: mutant PRPS2 stabilized the reporter as well as the wild-type construct. This indicates that PRPS2 likely functions as a stabilizer in non-catalytical manner (FIG. 6C).

KLHL40 belongs to the large BTB-BACK-Kelch domain family, and deletion constructs indicated that the BTB domain is both sufficient and necessary to stabilize the reporter in a proximity-dependent manner (FIG. 6D). Moreover, swapping the KLHL40 BTB domain with the same domain from a related family member KLHL6 (which is a robust degrader), turned KLHL40 into a degrader and KLHL6 into a stabilizer (FIG. 6E). In many BTB-BACK-Kelch domain proteins, the BTB domain interacts with CUL3, allowing them to function as substrate adapters for the CUL3-RING E3 ligase (CRL) complex. However, in KLHL40 the motif that interacts with Cul3 is not conserved (FIG. 6F), suggesting that it does not function as a part of a CRL complex. Interestingly, loss of KLHL40 in mice leads to destabilization of muscle intermediate filament proteins nebulin and LMOD3 (Garg et al., 2014; Ravenscroft et al., 2013). This finding together with these results strongly suggest that the endogenous function of KLHL40 is in protein stabilization and this activity can be re-targeted to non-physiological substrates through induced proximity.

### Effector Sensitivity to Tag Location

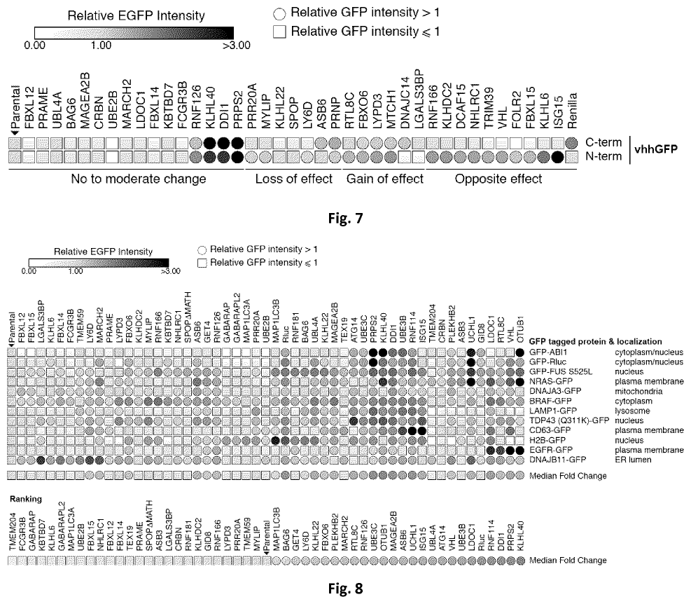

To test if the effectors identified in the screen are limited to specific geometries, 38 effectors were tagged with either N-terminal or C-terminal vhhGFP and assayed their activity in the original reporter cell line. While several effectors (such as KLHL22) only worked as C-terminal fusions, others (such as UBE2B, FBXL12 and FBXL14) were equally potent regardless of the tag location (FIG. 7). Similarly, DDI1 and KLHL40 stabilized the reporter as both C-terminal and N-terminal fusions.

### Specificity of Effectors Towards Different Substrates

Whether the top effectors were specific to the original reporter construct or whether they would be equally efficient with multiple different GFP-tagged proteins was also tested. To this end, 11 stable cell lines were generated expressing diverse GFP-tagged proteins localized to distinct cellular compartments and assayed the activity of YY effectors as vhhGFP fusions. The effectors showed strikingly different patterns, with some being highly potent across multiple targets while others affecting only a limited number of targets (FIG. 8). In FIG. 8 (bottom bar) illustrates that the effectors to the right of the Parental indication, from GET4 to KLHL40 are stabilizers and the effectors on the left of the Parental indication, starting from RNF166 to TMEM204 are degraders in the assay. These results establish that the screen did not reveal just effectors that target the GFP moiety of the reporter. Interestingly, CRBN was a potent degrader of almost all targets (with the exception of GFP-FUS and GFP-NRAS), whereas VHL showed very limited efficacy beyond the original GFP-ABI1 reporter. Notably, each target identified a unique complement of effectors that worked, including constructs that were localized to the same compartment (e.g., GFP-ABI1 and GFP-RLuc). These results indicate that proteins have strikingly different “preferences” for degraders and stabilizers, suggesting that expanding the toolbox of degraders would be highly beneficial for the development of next generation PROTACs and molecular glues. However, some effectors with a variety of target (for example UBE2B as degrader of a variety of target polypeptides). It is reasonable to expect that such robust effectors, such as UBE2B, would work as an effector with a variety of target polypeptides.

It was noticed that KLHL40, PRPS2, and DDI1 had a stabilizing effect on almost all tested targets (FIG. 8). It was therefore tested whether these effectors could stabilize proteins that are inherently unstable due to pathogenic mutations. Five different unstable variants representing different cellular compartments were selected and fused them to GFP-P2A-DsRed. The P2A motif induces ribosomal skipping during translation, which facilitates using DsRed as an internal control for protein stability. All three effectors stabilized the mutant variants more efficiently than Renilla luciferase (FIG. 9). Thus, these effectors represent novel classes of proximity-dependent stabilizers that could be harnessed in targeted protein stabilization.

Targeting Non-GFP Tagged and Endogenous Proteins with Novel Effectors

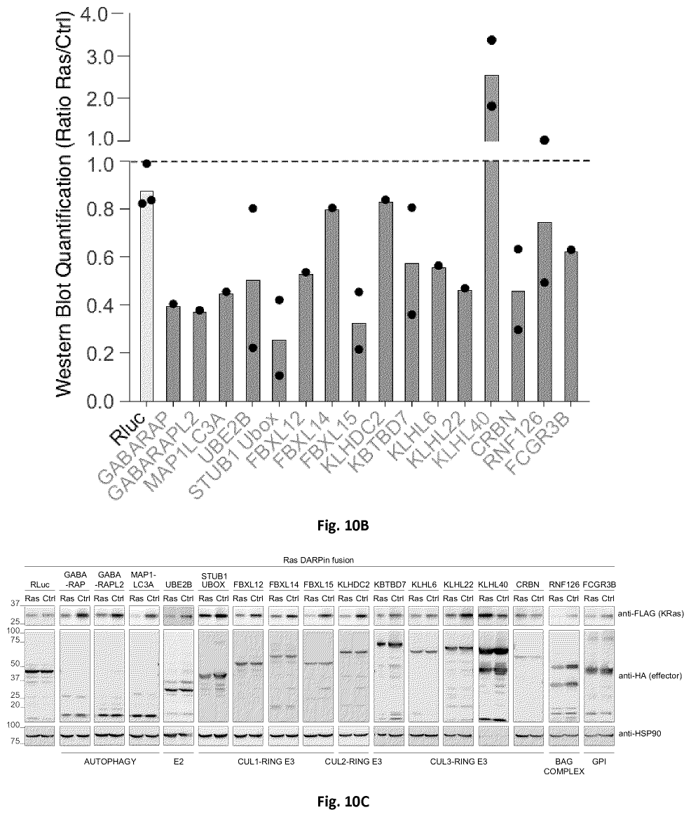

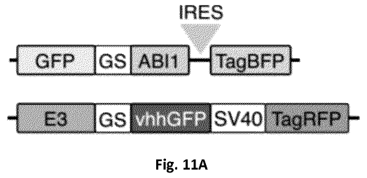

Until now, all the experiments were conducted with GFP tagged proteins, leaving open the possibility that the screen had identified effectors that target the GFP moiety instead of the protein fused to it. Therefore, whether the effectors are functional even when they are brought to the target by alternative means was tested. To do so, two previously developed RAS binders were used, an intracellular single domain antibody (iDab) and a designed ankyrin repeat protein (DARPin), which both bind KRAS with high affinity (Bery et al., 2019, 2020; Tanaka and Rabbitts, 2003). Due to the difficulty of detecting endogenous Ras, 3×FLAG-V5 tagged KRAS was co-transfected with the effectors fused to the binders. While Renilla luciferase fused to the binders had no effect on KRAS levels, most degradation effectors downregulated KRAS as iDab and DARPin fusions (FIGS. 10A and 10B). Notably, KLHL40 stabilized KRAS, consistent with its function with GFP tagged proteins. These results established that the effectors are not merely targeting GFP and that they are compatible with multiple different methods for induced proximity.

Finally, to test if the effectors can target endogenous proteins, several were fused to a monobody that binds the chromatin regulator WDR5 (Gupta et al., 2018). UBE2B, FBXL12, and KBTBD7 effectively decreased the levels of endogenous WDR5, indicating that these effectors can also target native proteins (FIG. 10C).

In summary, a collection of effector proteins that could be exploited in targeted protein degradation and stabilization approaches for therapeutic purposes were identified. These results indicate that these effectors (e.g. UBE2B) are more potent and less sensitive to geometry than existing TPD effectors, suggesting that they offer more versatility. Many of the newly identified effectors are not E3 ligases, suggesting that TPD could be expanded beyond this class of proteins.

### Systematic Analysis of E3 Ligases

Since many degraders and stabilizers were canonical E3 ligases, it was decided to test a large panel of E3 ligases by individually transfecting them as vhhGFP fusions into the original GFP-ABI1 cell line and assessing their effect on the reporter stability (FIG. 11A).

The two mainstay E3 ligases currently exploited in target protein degradation (TPD), the thalidomide target Cereblon (CRBN) and the tumor suppressor VHL appeared as robust degraders (FIG. 11B) and ranked among the top 20 degraders. Other Cullin-RING ubiquitin ligase (CRL) adaptors proteins also strongly induced GFP degradation (FIG. 11B). Degrader hits included the F-box domain proteins FBXL15, FBXO3, FBXO40, FBXO11, FBXW5, FBXL12, BTRC and FBXL14, in addition to several SOCS-box domain proteins, such as CISH and SOCS5. Other prominent CRL adaptor-type degraders included the BTB (POZ) domain proteins GMCL1, KBTBD7, KLHL12, GAN, KBTBD2, RHOBTB1 and KLHL6. In the contrary, transfection of the F-box domain proteins FBXL8, FBXO2, CDCA3 or SKP1 led to robust GFP stabilization. Similarly, GFP stabilization was also observed with the SOCS-box domain protein ASB9 and ELOB, and some of the BTB (POZ) domain proteins, such as KLHL40, ZFP161, KCTD17, ZBTB18, ZBTB7B, KCTD5, ZBTB20, ZBTB43, KEAP1, ZBTB10 and KLHL41.

Among the 137 E3s tested, 24 effectors reduced GFP intensity at least by half (relative GFP intensity <0.5), including TRIM31, RCHY1 and RNF166 (FIG. 1B). In contrast, none of the 9 tested HECT (Homologous to E6AP C-Terminus)-type E3s, induced GFP degradation. HECT family E3 ligases are often autoinhibited in the absence of a native substrate, possibly explaining this lack of activity in an induced proximity setting.

### Systematic Analysis of DUBs

In addition to E3 ligases, the effect of deubiquitinases on target protein stability was systematically analyzed. To identify potent DUBs, they were tested against an unstable disease variant GNMT H176N fused to GFP. 47 DUBs as vhhGFP fusions were co-transfected individually with the GNMT H176N-GFP construct and assessed their effect on the fusion protein stability (FIG. 12A). These DUBs represented all major families, including ubiquitin-specific proteases (USPs), ubiquitin C-terminal hydrolases (UCHs), ovarian tumor proteases (OTUs), Machado-Josephin domain proteases (MJDs), zinc-dependent metalloenzymes (JAMMs) and SUMO proteases. Interestingly, robust stabilizers across several different DUB families were found (FIG. 12B). The ubiquitin C-terminal hydrolases USP13, USP39, USP38 and USP14 were particularly potent at stabilizing the mutant construct, alongside with the ubiquitin C-terminal hydrolase UCHL1 and the ovarian tumor protease OTUB1.

### Characterization of Proximity-Dependent Stabilizers

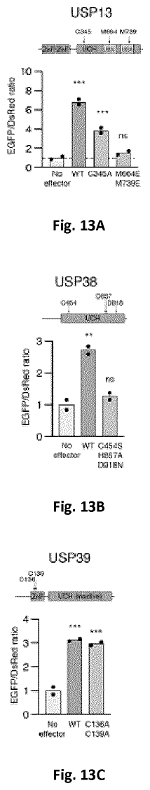

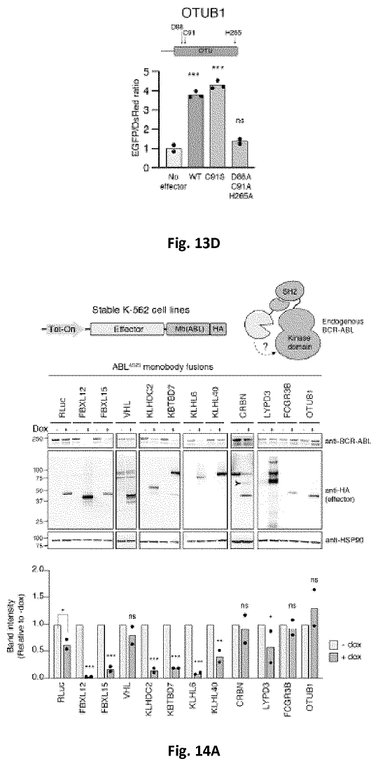

It was then tested if DUB catalytic activity was required for stabilization. Inactivating the catalytic cysteine of (USP13C345A) decreased the stabilizing effect of USP13, whereas disrupting the two ubiquitin-binding domains of USP13 (USP13M664E/M739E) abolished its activity on GNMTH176N (FIG. 13A). Catalytically dead mutant of USP3815 (USP38C454S/H857A/D918N) was similarly inactive in the assay (FIG. 13B). Thus, these two DUBs appear to function through a mechanism requiring catalytic activity and ubiquitin binding. In contrast, USP39 is a pseudoenzyme with no deubiquitinase activity in vitro. Moreover, mutating its ubiquitin-binding domain (USP39C136A/C139A) had no effect on activity (FIG. 13C), suggesting that USP39 functions in an alternative manner in this context. Similarly, OTUB1 catalytic activity was similarly dispensable (FIG. 13D). However, OTUB1 also has an additional non-catalytic function, as it can stoichiometrically inhibit the activity of E2s. A triple mutant construct deficient in E2 binding and inhibition was assayed and also predicted to be deficient in K48-linked ubiquitin chain binding. This mutant could not stabilize GNMTH176N (FIG. 13F), strongly suggesting that OTUB1 acts here via its E2 inhibiting function or its ability to bind to K48-linked ubiquitin chains.

FIGS. 13A-D show requirements for deubiquitinase function. For example, catalytic cysteine C91 is not required for OTUB1-mediated stabilization of the target protein, which was very unexpected. Indicated mutants of USP13 (FIG. 13A), USP38 (FIG. 13B), USP39 (FIG. 13C), and OTUB1 (FIG. 13D) were fused to vhhGFP and tested in the stabilization assay with GNMTH176N -EGFP. Statistical significance was calculated with one-way ANOVA with Dunnett's multiple comparison correction. *, p<0.05; **, p<0.01; ***, p<0.001).

### Recruiting Effectors Via Diverse Affinity Tags

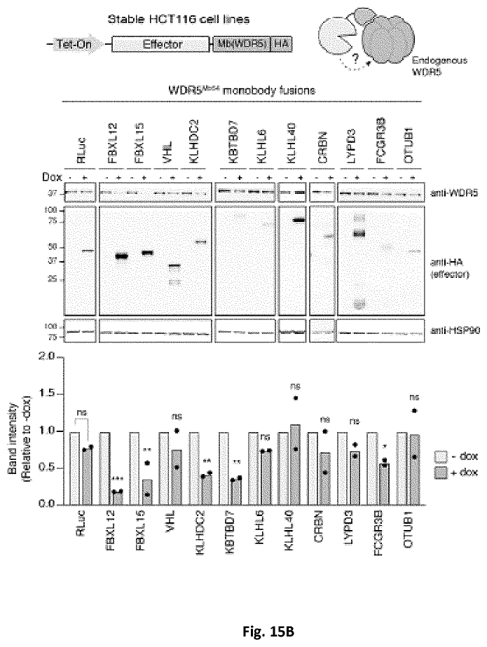

Recently, a comprehensive study revealed a striking difference in the susceptibility of kinases to 91 diverse PROTACs. Some kinases, such as ARAF and IKBKE, were not degraded by any compounds that engage VHL or CRBN. These two kinases were tagged with the 13-aa ALFA tag and selected effectors with the NbALFA nanobody. Consistent with the chemical proteomics approach, it was observed that VHL-NbALFA could not degrade either kinase in this assay (FIG. 15A). In contrast, many novel effectors were much more efficient. For example, FBXL12, FBXL15, KLHDC2 and the GPI-anchored protein FCGR3B potently lowered the levels of ARAF, whereas KLHL40 increased the levels (FIG. 15A).

Indicated effectors fused to Nb(ALFA)-Myc were co-transfected with ALFA-3×FLAG-ARAF into 293T cells, followed by western blotting for ARAF (anti-FLAG), effector (anti-Myc), and Hsp90 (FIG. 15A). Stable HCT116 cell lines expressing doxycycline-inducible effectors fused a WDR5-targeting monobody Mb(WDR5) were treated with doxycycline or left untreated (FIG. 15B). Endogenous WDR5 levels and effector expression were assessed by western blotting (FIG. 15B, Top). Quantification of WDR5 levels after doxycycline induction (FIG. 15B, Bottom). Statistical significance was calculated with an unpaired t-test with false discovery rate correction for multiple hypotheses. This provides further evidence that the top effectors can degrade hard-to-degrade cellular targets (like ARAF) or endogenous proteins (like WDR5).

Targeting Endogenous Proteins with Novel Effectors

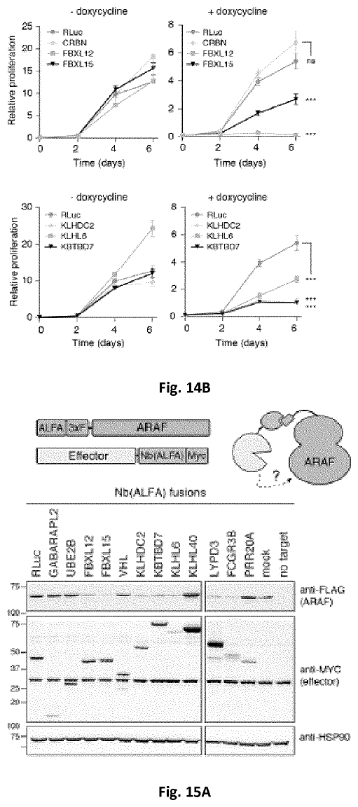

Finally, the potency of the effectors against two endogenous proteins, WDR5 and BCR-ABL were assessed. Selected effectors were cloned into an inducible lentiviral vector with a C-terminal fusion to specific WDR5 and BCR-ABL monobodies and generated stable HCT116 cells (for WDR5) and K562 cells (for BCR-ABL). While VHL could not degrade endogenous WDR5 and CRBN only had a modest effect, several novel effectors robustly degraded WDR5 in a doxycycline-dependent manner (FIG. 15B). In particular, FBXL12 and FBXL15 were, again, highly efficient. The results with BCR-ABL in K562 cells were similar: FBXL12, FBXL15, KBTBD7, KLHDC2, and KLHL6 degraded BCR-ABL very potently, whereas CRBN and VHL had no significant effect (FIG. 14A).

Because BCR-ABL is an essential protein in K562 cells, the effect of degrader fusions on cell proliferation was then assessed. Although the monobody alone inhibits the function of BCR-ABL101, fusing it to an inert control, RLuc, only partially inhibited the proliferation of K562 cells (FIG. 14B). However, when the monobody was fused to novel effectors, the cells completely ceased proliferation or proliferated significantly slower (FIG. 14B). In contrast, CRBN did not further affect proliferation (FIG. 14B). Thus, many effectors identified in the unbiased ORFeome screen are significantly better at degrading and inhibiting the function of the hallmark oncogenic fusion of chronic myeloid leukemia, BCR-ABL.

FIGS. 14A-B depict the results of benchmarking novel effectors with multiple recruitment strategies and therapeutically relevant targets. In particular, FIGS. 14A-B show the potency of top effectors in degrading endogenous BCR-ABL and inhibiting cell growth. Stable K-562 cell lines expressing doxycycline-inducible effectors fused to a monobody binding the SH2 domain of BCR-ABL were treated with doxycycline or left untreated (FIG. 14A). Endogenous BCR-ABL levels and effector expression were assessed by western blotting (FIG. 14A, Top). FIG. 14A, Bottom depicts the quantification of BCR-ABL levels after doxycycline induction. Statistical significance was calculated with an unpaired t-test with false discovery rate correction for multiple hypotheses. FIG. 14B depicts K562 cell proliferation after induction of indicated effector fusion constructs with doxycycline. The monobody itself has an effect on cell proliferation (compare top left graph scale to top right graph for RLuc-vhhGFP (FIG. 14B)).

### Example 2

### Viability Screening Assays

First, a target polypeptide of interest (such as KRAS) would be tagged with GFP or ABI1 (or other tags) so that it's the only source of the protein in the cell. That would be achieved either by tagging the endogenous locus or by ectopically expressing the tagged version and knocking out the endogenous copy. Then putative effector polypeptides comprised in a collection, for example a library (ex. ORFeome), fused to a targeting moiety that binds the target polypeptide or the tag fused to the target polypeptide (for example, vhhGFP, PYL1 or other antibodies), would be screened. Then, putative effector polypeptides that disappear from the collection over time would be identified. The identification of putative effectors that disappear during the screening assay would be done for example by sequencing the DNA encoding the putative effector polypeptides present in the cells that survived to determine which putative effector polypeptides are or are not present in such cells by comparing the effector genes present in the original collection with the effector genes present after screening. This would indicate that the putative effector polypeptide's interaction with the target polypeptide is lethal to the cell. These factors could include effector polypeptides that degrade the target, but also other effector polypeptides that inhibit target function by other means.

Alternatively, this method could be performed without adding a tag to the target polypeptide and the putative effector polypeptides could be fused to a nanobody or another targeting moiety which binds the target polypeptide and the screen would be conducted against the endogenous target polypeptide, which would not be tagged.

### Example 3

### Protein Trafficking Screening Assays

First, a cell surface protein (such as CFTR delta508) would be tagged with GFP, ABI1 or other tags. Then, putative effector polypeptides comprised in a collection, for example a library would be screened for putative effector polypeptides that increase the cell surface localization of the target polypeptide (the cell surface protein). This could be done with FACS either by using an antibody against an extracellular epitope of the protein or by adding another epitope (like FLAG tag) to an extracellular part of the protein. Sequencing clones after FACS sorting for high surface expression would identify those putative effector polypeptides that promote trafficking.

Alternatively, this method could be performed without adding a tag to the target polypeptide and the putative effector polypeptides could be fused to a nanobody or another targeting moiety which binds the target polypeptide and the screen would be conducted against the endogenous target polypeptide, which would not be tagged.

### Example 4

### Indirect Proximity Interaction Screen

The effectors do not need to be directly fused to the proximity-inducing protein such as vhhGFP or PYL1. For example, they could be fused to a small tag that promotes interaction with a secondary factor, which interacts with a target. Such examples are SpyTag/SpyCatcher, SnoopTag/SnoopCatcher, HiBiT/LgBit, or GFP11/GFP1-10. SpyCatcher and SnoopCatcher are proteins that will form a covalent bond with SpyTag and SnoopTag peptides, respectively. LgBit and GFP1-10 are fragments of Nanoluc luciferase and GFP, which bind to HiBiT and GFP11 peptides, respectively, with high affinity. Effectors could be brought to the target by fusing them to SpyTag in cells that express SpyCatcher that is fused to PYL1 or another moiety that induces interaction with the target. Thus, effectors would be induced to interact with the target with the help of a secondary factor (SpyCatcher-PYL1 fusion in this case).

### Alternative Readouts (not Fluorescent Protein Fusion)

In addition to a fluorescent protein, the target could be fused to a small epitope tag (such as FLAG, V5, Myc, ALFA or HA) and its abundance detected with an epitope tag antibody. Alternatively, a completely untagged (endogenous) target could be detected with an antibody against the target, using flow cytometer. In these cases, the effectors would be brought to the target using nanobodies, ScFv fragments, monobodies, affibodies or similar affinity reagents against the epitope tag or against the target itself.

For the antibiotic selection screen, the target would be fused to an antibiotic resistance marker (e.g., puromycin N-acetyltransferase or blasticidin deaminase) or a negative selection marker (e.g. thymidine kinase or deoxycytidine kinase DCK*). In the first case, effectors that increase the level of the target would increase the levels of the antibiotic resistance marker. These cells would then be more resistant to the antibiotic, facilitating the discovery of stabilizing effectors. In the second case, effectors that decrease the target levels would also decrease the levels of the negative selection marker. Cells that express thymidine kinase are sensitive to ganciclovir, whereas those that express mutant deoxycytidine kinase (DCK*) are sensitive to 2-bromovinyldeoxyuridine (BVdU). Treating the cell population with these compounds would select for effectors that degrade the target.

### Example 5

### Robust Effectors

The methods described in Examples 1 and 2, identified a number of robust degraders and stabilizers. For example, the following group were identified that had comparable or increased activity compared to CRBN or VHL:
GMCL1, FBXL15, PJA1, RNF115, DZIP3, RNF125, FBXO3, RNF185, RNF8, RNF183, RCHY1, KBTBD7, TRIM31, CISH, SOCS5, TRIM39, RNF144B, FBXO40, KLHL6, FBXO11, GAN, FBXL14, FBXW5, RNF111, FBXL12, BTRC, RNF126. ZER1 also has increased activity compared to CRBN or VHL.

TABLE 6
Robust effectors

| | UniProt Accession | Degrader/ | E3 ligase |
|---|---|---|---|
| Protein | Number or SEQ ID NO | Stabilizer | type |
| SOCS5 | O75159 | Degrader | E3 (CRL |
| | | | adaptor) |
| RNF8 | O76064 | Degrader | RING-E3 |
| UBE2B | P63146 | Degrader | E2 |
| | | | conjugating |
| | | | enzyme |
| RNF111 | Q6ZNA4 | Degrader | RING-E3 |
| RNF144B | Q7Z419 | Degrader | RING-E3 |
| FBXO11 | Q86XK2 | Degrader | E3 (CRL |
| | | | adaptor) |
| DZIP3 | Q86Y13 | Degrader | RING-E3 |
| FBXL14 | Q8N1E6 | Degrader | E3 (CRL |
| | | | adaptor) |
| PJA1 | Q8NG27 | Degrader | RING-E3 |
| KBTBD7 | Q8WVZ9 | Degrader | E3 (CRL |
| | | | adaptor) |
| KLHL6 | Q8WZ60 | Degrader | E3 (CRL |
| | | | adaptor) |
| FBXW5 | Q969U6 | Degrader | E3 (CRL |
| | | | adaptor) |
| RNF183 | Q96D59 | Degrader | RING-E3 |
| RNF125 | Q96EQ8 | Degrader | RING-E3 |
| RNF185 | Q96GF1 | Degrader | RING-E3 |
| GMCL1 | Q96IK5 | Degrader | E3 (CRL |
| | | | adaptor) |
| RCHY1 | Q96PM5 | Degrader | RING-E3 |
| RNF126 | Q9BV68 | Degrader | RING-E3 |
| TRIM31 | Q9BZY9 | Degrader | RING-E3 |
| GAN | Q9H2C0 | Degrader | E3 (CRL |
| | | | adaptor) |
| FBXL15 | Q9H469 | Degrader | E3 (CRL |
| | | | adaptor) |
| TRIM39 | Q9HCM9 | Degrader | RING-E3 |
| CISH | Q9NSE2 | Degrader | E3 (CRL |
| | | | adaptor) |
| FBXL12 | Q9NXK8 | Degrader | E3 (CRL |
| | | | adaptor) |
| FBXO40 | Q9UH90 | Degrader | E3 (CRL |
| | | | adaptor) |
| FBXO3 | Q9UK99 | Degrader | E3 (CRL |
| | | | adaptor) |
| BTRC | Q9Y297 | Degrader | E3 (CRL |
| | | | adaptor) |
| KLHDC2 | Q9Y2U9 | Degrader | E3 (CRL |
| | | | adaptor) |
| RNF115 | Q9Y4L5 | Degrader | RING-E3 |

### Example 6

Discovery of Small-Molecule Ligands/Binders for Effector Proteins and their Targets

There are multiple options to identify binders to an effector or a target. In most cases, recombinant effector and/or target protein is first purified, followed by chemical screens.

Several screening methods can be employed to identify small-molecule binders without considering their mechanism of action (e.g., inhibition or activation). These methods typically focus on measuring binding interactions or changes in protein properties upon ligand binding. Some of these techniques include:

Surface Plasmon Resonance (SPR): SPR measures the binding affinity and kinetics of small molecules interacting with their target proteins in a label-free manner, without requiring information about their mechanism of action.

Nuclear Magnetic Resonance (NMR) spectroscopy: NMR can detect changes in a protein's spectrum upon binding to a small molecule, allowing the identification of binders without the need to know their mode of action.

Differential Scanning Fluorimetry (DSF) or Thermal Shift Assay (TSA): By monitoring the target protein's thermal stability upon small-molecule binding, this method can identify binders without prior knowledge of their functional effects.

Isothermal Titration Calorimetry (ITC): ITC measures the heat generated or absorbed upon the interaction between small molecules and their target proteins, providing binding affinity and stoichiometry data without specifying the mechanism of action.

Microscale Thermophoresis (MST): MST detects changes in the movement of a fluorescently labeled target protein in a temperature gradient upon binding to a small molecule, providing information on the interaction without considering the mode of action.

Biolayer Interferometry (BLI): This label-free optical technique measures the change in interference patterns caused by small molecules binding to immobilized target proteins, without requiring information about the mechanism of action.

X-ray Crystallography: This method can determine the three-dimensional structure of protein-small molecule complexes, providing insights into molecular interactions and binding modes, regardless of the mechanism of action.

DNA-Encoded Library (DEL) screens: An innovative screening approach where each small molecule in the library is covalently attached to a unique DNA barcode. This allows for the parallel screening of vast compound libraries, and binders can be identified by amplifying and sequencing the DNA barcodes of bound molecules.

Affinity Selection-Mass Spectrometry (AS-MS): This technique involves incubating a target protein with a compound library, followed by affinity purification to isolate protein-bound small molecules. The bound molecules are then identified using mass spectrometry. AS-MS can detect binders without prior knowledge of their mechanism of action.

Covalent Fragment Screens: A specialized form of fragment-based screening that focuses on identifying small molecules that form covalent bonds with the target protein. This approach can identify binders regardless of their mechanism of action, as it primarily detects covalent interactions rather than functional effects.

These techniques focus on detecting binding events or changes in protein properties upon ligand binding, which allows them to identify binders without prior knowledge of their functional effects. After identifying potential binders, additional assays can be performed to characterize their mechanisms of action, such as activation or inhibition.

If there is no ligand for the effector nor the target, one would do two separate screens to find them. This would be followed by medicinal chemistry effort to connect the identified binders with various linkers and characterize these heterobifunctional molecules for their ability to induce an interaction between the target and the effector.

One could use several approaches for de novo discovery of molecular glues, such as luciferase complementation (each protein has one half of a luciferase, interaction leads to luminescence), yeast two-hybrid assay using reporter genes, AlphaScreen (proximity-based luminescence/fluorescence assay), yeast mating based interaction assay (SynAg), FRET or TR-FRET.

### Example 7

### Materials and Methods

Materials and methods are as described in Example 1 or as described herein. Cell lines

HeLa Kyoto, HCT116, and all 293T cells, including the EGFP-ABI1-IRES-TagBFP reporter cellline used for screens, were maintained in DMEM supplemented with 10% fetal bovine serum (FBS) and 1% penicillin-streptomycin. K562 cells were cultured in RPMI supplemented with 10% fetal bovine serum (FBS) and 1% penicillin-streptomycin. 293T, K562, and HCT116 Tet-inducible cell lines were maintained in their respective regular media supplemented with 10% Tet system approved FBS (Gibco A47363-01) and 1% penicillin-streptomycin. Cells were maintained at 37° C. in a humidified incubator at 5% C02 and routinely tested for mycoplasma contamination.

### Plasmids

Unstable mutant targets were cloned into pcDNA3.1-[ORF]-GSlinker-EGFP-P2A-DsRed destination vector, using Gateway cloning technology. For the vhhGFP and PYL1 degradation assays, entry clones were picked from the hORFeome collection and subcloned into pcDNA3.1-[ORF]-GSlinker-vhhGFP-SV40-TagBFP, pcDNA3.1-[ORF]-GSlinker-vhhGFP, pcDNA3.1-vhhGFP-ORF and pcDNA3.1-[ORF]-GSlinker-PYL1 destination vectors. For WDR5 endogenous protein degradation, effector-coding sequences were cloned into pSTV6-TetO-[ORF]-Mb(S4) WDR5-HA lentiviral plasmid, allowing expression of the respective proteins with a C-terminal monobody Mb(S4) recognizing WDR5 with a high affinity. For BCR-ABL endogenous protein degradation, effector coding sequences were cloned into pSTV6-TetO-[ORF]-AS25-HA lentiviral vector, allowing expression of the respective proteins with a C-terminal high affinity monobody AS25 directed to the Src homology 2 (SH2)-kinase domain interaction interface. To generate ALFA tagged ARAF or IKBKE, their open reading frames were cloned into the Gateway-compatible pcDNA3.4-ALFA-3×FLAG-ORF and pcDNA3.4-[ORF]-3×FLAG-ALFA vectors, respectively. ALFA nanobody fused effectors were generated by subcloning each effector gene into the Gateway compatible pcDNA3.4-[ORF]-NbALFA-Myc plasmid.

For ectopic K-Ras expression, KRAS cDNA was cloned into pcDNA3.1-3×FLAG-ORF destination vector. The effector coding sequences were cloned into pcDNA3.4-[ORF]-Ras iDab-HA and pcDNA3.1-[ORF]-LMO2 iDab-HA vectors, allowing expression of the respective proteins with a C-terminal monobody recognizing K-Ras or a control protein (LMO2), respectively.

Point mutants were generated by site-directed mutagenesis and deletion constructs were generated by PCR.

### Stable Cell Line Generation

A monoclonal 293T cell line expressing EGFP-ABI1-IRES-TagBFP was generated by sorting single cells by FACS after lentiviral infection and blasticidin (6 μg/ml) selection. A clone showing high EGFP and TagBFP expression was selected for subsequent experiments. To generate the inducible doxycycline-inducible cell lines, effectors were subcloned into Gateway compatible pSTV6-TetO-[ORF]-EGFP, pSTV6-TetO-[ORF]-AS25-HA or pSTV6-TetO-[ORF]-Mb(S4) WDR5-HA lentiviral plasmids. 293T and HCT116 cells were infected in the presence of 8 μg/mL polybrene and selected with 3 μg/ml puromycin 24 hours post infection. K562 cells were infected by spin-down at 3000 rpm for 90 minutes in the presence of 8 μg/ml polybrene and selected with 3 μg/ml puromycin 24 hours post infection.

To enrich for high EGFP expression with fusion proteins, cells infected with pSTV6-TetO-[ORF]-EGFP lentivirus were induced with 1 μg/ml doxycycline and sorted for high EGFP population.

Analysis of Sequencing Data from Pooled Activation Screens

An index of the ORFeome reference sequences was created using the STAR aligner v2.7.8a. Reads from the ORFeome libraries were aligned with the STAR aligner allowing a maximum of 3 mismatches. To identify degradation and stabilization effectors, the edgeR package was used to calculate log 2 fold change, p-value, and false discovery rate (FDR) for each ORF by comparing changes in counts from sorted samples to unsorted cells. In the degradation screen, a 5% false discovery rate cut-off and 4-fold change in normalized read counts between bottom 10% vs unsorted cells was used. In the stabilization screen, a 1% FDR cut-off and 4-fold change in normalized read counts was used.

### K-Ras Degradation Assay

HeLa cells were seeded in 12-well plates (100,000 cells/well) and transfected 24 hours later with 200 ng of 3×FLAG-KRAS and 400 ng of effector fused to Ras iDab or LMO2 iDab 48h hours after transfection, cells were harvested and subjected to western blot analysis.

### Endogenous Protein Degradation Assay

K562 and HCT116 stable cell lines were seeded in 12-well plates (1×106 cells/well). K562 cells were incubated with 1 μg/ml of doxycycline or 1% DMSO for 48 hours. HCT116 cells were incubated for 24 hours to let the cells acclimatize before being treated with 1 μg/ml of doxycycline or 1% DMSO for another 24 hours. Cells were then harvested and subjected to western blot analysis.

### ALFA-Tagged Kinase Degradation Assay

ALFA-tagged kinase degradation assays were performed in a 12-well cell culture format (100,000 cells/well). After 24 hours, 293T cells were transiently transfected with 1 μg of either pcDNA3.4-ALFA-3×FLAG-TEV-ARAF or pcDNA3.4-IKBKE-3×FLAG-ALFA, using Lipofectamine 2000 (Life Technologies) following the manufacturer's instructions. Cells were harvested and subjected to western blot analysis 16 hours post-transfection.

### Western Blot

Cells were lysed in CSK lysis buffer (20 mM Hepes-KOH pH 7.9, 100 mM NaCl, 1 mM MgCl2, 1 mM EDTA, 300 mM sucrose, 1 mM DTT, 0.1% Triton X-100, benzonase, and protease inhibitor cocktail). For BCR-ABL, WDR5, ARAF and IKBKE degradation assay, cells were lysed in NP40 lysis buffer (50 mM Tris-HCl pH 7.6, 150 mM NaCl, 1% NP40 and protease inhibitor cocktail). After centrifugation at 16,000 g for 5 min at 4° C., the same amount of each cellular lysate was analyzed by gel electrophoresis and western blot using anti-WDR5 antibody (D9E11; Cell Signaling #13105), anti-HSP90 antibody (F-8; Santa Cruz Biotechnology), anti-HA (Sigma #H6908), anti-c-ABL (Cell Signaling #2862T), anti-MYC (BioLegend #626802), and anti-FLAG (DSHB #12C6c) as the primary antibody. Goat HRP-conjugated anti-rabbit IgG (Cell Signaling #7074S) or anti-mouse IgG (Cell Signaling #7076S) were used as secondary antibodies. MonoRab™ HRP Rabbit anti-Camelid VHH antibody (GenScript #A01861) was used to detect vhhGFP fusion proteins. Chemiluminescence signal was generated with Immobilon Western Chemiluminescent HRP Substrate (Millipore) and detected with MicroChemi4.2 (FroggaBio).

### Proliferation Assay of Stable K562 Cell Lines

Proliferation assay was performed in a 96-well culture format (1,000 cells/well). Cells were grown in the presence of 1 μg/ml doxycycline or 1% DMSO for the indicated times. Doxycycline or DMSO were newly added after 72 hours. CellTiter Glo (Promega) was used to measure cell viability, following the manufacturer's instructions. Luminescence intensities were measured using a multimode microplate reader (Biotek).

### Immunofluorescence

HeLa Kyoto cell were seeded into opaque black, clear bottom 96-well plates at 4,500-5,000 cells per well. The next day, cells were transfected using XtremeGENE 9 (Roche), as per the manufacturer's instructions. 48 hours after transfection, cells were washed with 1×PBS and then fixed for 15 minutes at room temperature with 4% paraformaldehyde in DMEM containing 10% FBS. Following fixation, cells were washed three times in 1×PBS, permeabilized with 0.1% Triton X-100/1×PBS, and then blocked with blocking buffer (0.1% Triton X-100/1×PBS/1% BSA) for 30 minutes at room temperature. After blocking, fixed cells were incubated with MonoRab™ iFluor 647 Rabbit Anti-Camelid VHH antibody (GenScript A01994) and Hoechst 33342 diluted in blocking buffer for 1 hour at room temperature. Finally, cells were washed three times in 1×PBS and imaged using the Opera Phenix high-content microscope (Perkin Elmer) at 63× magnification.

### Results

### Characterization of Proximity-Dependent Stabilizers

It was then tested if DUB catalytic activity was required for stabilization. Inactivating the catalytic cysteine of (USP13C345A) decreased the stabilizing effect of USP13, whereas disrupting the two ubiquitin-binding domains of USP13 (USP13M664E/M739E) abolished its activity on GNMTH176N (FIG. 13A). Catalytically dead mutant of USP3815 (USP38C454S/H857A/D918N) was similarly inactive in the assay (FIG. 13B). Thus, these two DUBs appear to function through a mechanism requiring catalytic activity and ubiquitin binding. In contrast, USP39 is a pseudoenzyme with no deubiquitinase activity in vitro. Moreover, mutating its ubiquitin-binding domain (USP39C136A/C139A) had no effect on activity (FIG. 13C), suggesting that USP39 functions in an alternative manner in this context. Similarly, OTUB1 catalytic activity was similarly dispensable (FIG. 13D). However, OTUB1 also has an additional non-catalytic function, as it can stoichiometrically inhibit the activity of E2s. A triple mutant construct deficient in E2 binding and inhibition was assayed and also predicted to be deficient in K48-linked ubiquitin chain binding. This mutant could not stabilize GNMTH176N (FIG. 13F), strongly suggesting that OTUB1 acts here via its E2 inhibiting function or its ability to bind to K48-linked ubiquitin chains.

FIGS. 13A-D show requirements for deubiquitinase function. For example, catalytic cysteine C91 is not required for OTUB1-mediated stabilization of the target protein, which was very unexpected. Indicated mutants of USP13 (FIG. 13A), USP38 (FIG. 13B), USP39 (FIG. 13C), and OTUB1 (FIG. 13D) were fused to vhhGFP and tested in the stabilization assay with GNMTH176N -EGFP. Statistical significance was calculated with one-way ANOVA with Dunnett's multiple comparison correction. *, p<0.05; **, p<0.01; ***, p<0.001).

### Recruiting Effectors Via Diverse Affinity Tags

Recently, a comprehensive study revealed a striking difference in the susceptibility of kinases to 91 diverse PROTACs. Some kinases, such as ARAF and IKBKE, were not degraded by any compounds that engage VHL or CRBN. These two kinases were tagged with the 13-aa ALFA tag and selected effectors with the NbALFA nanobody. Consistent with the chemical proteomics approach, it was observed that VHL-NbALFA could not degrade either kinase in this assay (FIG. 15A). In contrast, many novel effectors were much more efficient. For example, FBXL12, FBXL15, KLHDC2 and the GPI-anchored protein FCGR3B potently lowered the levels of ARAF, whereas KLHL40 increased the levels (FIG. 15A).

Indicated effectors fused to Nb(ALFA)-Myc were co-transfected with ALFA-3×FLAG-ARAF into 293T cells, followed by western blotting for ARAF (anti-FLAG), effector (anti-Myc), and Hsp90 (FIG. 15A). Stable HCT116 cell lines expressing doxycycline-inducible effectors fused a WDR5-targeting monobody Mb(WDR5) were treated with doxycycline or left untreated (FIG. 15B). Endogenous WDR5 levels and effector expression were assessed by western blotting (FIG. 15B, Top). Quantification of WDR5 levels after doxycycline induction (FIG. 15B, Bottom). Statistical significance was calculated with an unpaired t-test with false discovery rate correction for multiple hypotheses. This provides further evidence that the top effectors can degrade hard-to-degrade cellular targets (like ARAF) or endogenous proteins (like WDR5).

Targeting Endogenous Proteins with Novel Effectors

Finally, the potency of the effectors against two endogenous proteins, WDR5 and BCR-ABL were assessed. Selected effectors were cloned into an inducible lentiviral vector with a C-terminal fusion to specific WDR5 and BCR-ABL monobodies and generated stable HCT116 cells (for WDR5) and K562 cells (for BCR-ABL). While VHL could not degrade endogenous WDR5 and CRBN only had a modest effect, several novel effectors robustly degraded WDR5 in a doxycycline-dependent manner (FIG. 15B). In particular, FBXL12 and FBXL15 were, again, highly efficient. The results with BCR-ABL in K562 cells were similar: FBXL12, FBXL15, KBTBD7, KLHDC2, and KLHL6 degraded BCR-ABL very potently, whereas CRBN and VHL had no significant effect (FIG. 14A).

Because BCR-ABL is an essential protein in K562 cells, the effect of degrader fusions on cell proliferation was then assessed. Although the monobody alone inhibits the function of BCR-ABL101, fusing it to an inert control, RLuc, only partially inhibited the proliferation of K562 cells (FIG. 14B). However, when the monobody was fused to novel effectors, the cells completely ceased proliferation or proliferated significantly slower (FIG. 14B). In contrast, CRBN did not further affect proliferation (FIG. 14B). Thus, many effectors identified in the unbiased ORFeome screen are significantly better at degrading and inhibiting the function of the hallmark oncogenic fusion of chronic myeloid leukemia, BCR-ABL.

FIGS. 14A-B depict the results of benchmarking novel effectors with multiple recruitment strategies and therapeutically relevant targets. In particular, FIGS. 14A-B show the potency of top effectors in degrading endogenous BCR-ABL and inhibiting cell growth. Stable K-562 cell lines expressing doxycycline-inducible effectors fused to a monobody binding the SH2 domain of BCR-ABL were treated with doxycycline or left untreated (FIG. 14A). Endogenous BCR-ABL levels and effector expression were assessed by western blotting (FIG. 14A, Top). FIG. 14A, Bottom depicts the quantification of BCR-ABL levels after doxycycline induction. Statistical significance was calculated with an unpaired t-test with false discovery rate correction for multiple hypotheses. FIG. 14B depicts K562 cell proliferation after induction of indicated effector fusion constructs with doxycycline. The monobody itself has an effect on cell proliferation (compare top left graph scale to top right graph for RLuc-vhhGFP (FIG. 14B)).

TABLE 7
List of proximity effector polypeptides identified in both
the degradation screen using vhhGFP fused to the ORFeome
ORFs, and the degradation screen using PYL1 fused to ORFeome
ORFs (see FIG. 1A and Table 4 for Accession Numbers)

| Effector | |
|---|---|
| UPS-related | Transport |
| FBXL12 | NRNS1 |
| GID8 | NRNS2 |
| KBTBD7 | PLEKHB2 |
| KLHDC2 | SLC37A1 |
| KLHL11 | TTYH2 |
| KLHL6 | TTYH3 |
| LAPTM5 | Other |
| TEX19 | NATD1 |
| UBE2B | CXCR7 |
| ZER1 | DLK1 |
| GPI-Anchored | EID1 |
| GPC1 | IGSF21 |
| FCGR3B | PRR20A |
| FOLR1 | TMEM171 |
| FOLR2 | VTN |
| LYPD3 | |
| THY1 | |

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g., protein and/or nucleic acid) provided in the Tables or elsewhere, are incorporated by reference in its entirely.

The scope of the claims should not be limited by the preferred embodiments and examples but should be given the broadest interpretation consistent with the description as a whole.

### REFERENCES

Bery, N., Legg, S., Debreczeni, J., Breed, J., Embrey, K., Stubbs, C., Kolasinska-Zwierz, P., Barrett, N., Marwood, R., Watson, J., et al. (2019). KRAS-specific inhibition using a DARPin binding to a site in the allosteric lobe. Nat Commun 10, 2607.
Bery, N., Miller, A., and Rabbitts, T. (2020). A potent KRAS macromolecule degrader specifically targeting tumours with mutant KRAS. Nat Commun 11, 3233.
Burslem, G. M., and Crews, C. M. (2020). Proteolysis-Targeting Chimeras as Therapeutics and Tools for Biological Discovery. Cell 181, 102-114.
Caussinus, E., Kanca, O., and Affolter, M. (2012). Fluorescent fusion protein knockout mediated by anti-GFP nanobody. Nature Structural & Molecular Biology 19, 117-121.
David, Y., Ziv, T., Admon, A., and Navon, A. (2010). The E2 Ubiquitin-conjugating Enzymes Direct Polyubiquitination to Preferred Lysines. Journal of Biological Chemistry 285, 8595-8604.
Dowling, J. J., Weihl, C. C. & Spencer, M. J. Molecular and cellular basis of genetically inherited skeletal muscle disorders. Nat Rev Mol Cell Biol 22, 713-732 (2021).
Garg, A., O'Rourke, J., Long, C., Doering, J., Ravenscroft, G., Bezprozvannaya, S., Nelson, B. R., Beetz, N., Li, L., Chen, S., et al. (2014). KLHL40 deficiency destabilizes thin filament proteins and promotes nemaline myopathy. J Clin Invest 124, 3529-3539.
Gerry, C. J., and Schreiber, S. L. (2020). Unifying principles of bifunctional, proximity-inducing small molecules. Nat Chem Biol 16, 369-378.
Gupta, A., Xu, J., Lee, S., Tsai, S. T., Zhou, B., Kurosawa, K., Werner, M. S., Koide, A., Ruthenburg, A. J., Dou, Y., et al. (2018). Facile target validation in an animal model with intracellularly expressed monobodies. Nat Chem Biol 14, 895-900.
Henning, N.J., Boike, L., Spradlin, J. N., Ward, C. C., Belcher, B., Brittain, S. M., Hesse, M., Dovala, D., McGregor, L. M., McKenna, J. M., et al. (2021). Deubiquitinase-Targeting Chimeras for Targeted Protein Stabilization (Biochemistry).
Ju, J.-S., Fuentealba, R. A., Miller, S. E., Jackson, E., Piwnica-Worms, D., Baloh, R. H., and Weihl, C. C. (2009). Valosin-containing protein (VCP) is required for autophagy and is disrupted in VCP disease. Journal of Cell Biology 187, 875-888.
Kobayashi, H., Shoji, K., Kiyokawa, K., Negishi, L., and Tomari, Y. (2019). VCP Machinery Mediates Autophagic Degradation of Empty Argonaute. Cell Reports 28, 1144-1153.e4.
Lei, Y., and Klionsky, D. J. (2021). New functions of a known autophagy regulator: VCP and autophagy initiation. Autophagy 17, 1063-1064.
Liang, F.-S., Ho, W. Q., and Crabtree, G. R. (2011). Engineering the ABA plant stress pathway for regulation of induced proximity. Science Signaling 4, rs2-rs2.
Neschadim, Anton et al. “Cell fate control gene therapy based on engineered variants of human deoxycytidine kinase.” Molecular therapy: the journal of the American Society of Gene Therapy vol. 20, 5 (2012): 1002-13.
Nguyen, T. V., Li, J., Lu, C.-C. (Jean), Mamrosh, J. L., Lu, G., Cathers, B. E., and Deshaies, R. J. (2017). p97/VCP promotes degradation of CRBN substrate glutamine synthetase and neosubstrates. PNAS 114, 3565-3571.
Ravenscroft, G., Miyatake, S., Lehtokari, V.-L., Todd, E. J., Vornanen, P., Yau, K. S., Hayashi, Y. K., Miyake, N., Tsurusaki, Y., Doi, H., et al. (2013). Mutations in KLHL40 are a frequent cause of severe autosomal-recessive nemaline myopathy. Am J Hum Genet 93, 6-18.
Reitsma, J. M., Liu, X., Reichermeier, K. M., Moradian, A., Sweredoski, M. J., Hess, S., and Deshaies, R. J. (2017). Composition and Regulation of the Cellular Repertoire of SCF Ubiquitin Ligases. Cell 0, 1326-1339.e14.
Robinson, M. D., McCarthy, D. J. and Smyth, G. K. (2010). edgeR: a bioconductor package for differential expression analysis of digital gene expression data. Bioinformatics 26, 139-140.
Saerens, D., Pellis, M., Loris, R., Pardon, E., Dumoulin, M., Matagne, A., Wyns, L., Muyldermans, S., and Conrath, K. (2005). Identification of a Universal VHH Framework to Graft Non-canonical Antigen-binding Loops of Camel Single-domain Antibodies. Journal of Molecular Biology 352, 597-607.
Stewart, M. D., Ritterhoff, T., Klevit, R. E., and Brzovic, P. S. (2016). E2 enzymes: more than just middle men. Cell Res 26, 423-440.
Takahashi, D., Moriyama, J., Nakamura, T., Miki, E., Takahashi, E., Sato, A., Akaike, T., Itto-Nakama, K., and Arimoto, H. (2019). AUTACs: Cargo-Specific Degraders Using Selective Autophagy. Mol Cell 76, 797-810.e10.
Tanaka, T., and Rabbitts, T. H. (2003). Intrabodies based on intracellular capture frameworks that bind the RAS protein with high affinity and impair oncogenic transformation. EMBO J 22, 1025-1035.
Tanaka, T., Williams, R. H., and Rabbitts, T. H. (2007). Tumour prevention by a single antibody domain targeting the interaction of signal transduction proteins with RAS. EMBO J 26, 3250-3259.
Tanaka, T., Sewell, H., Waters, S., Phillips, S. E. V. and Rabbitts, T. H. (2011). Single domain intracellular antibodies from diverse libraries. Immunology 286, 3707-3716.
Watanabe, K., Yumimoto, K., and Nakayama, K. I. (2015). FBXO21 mediates the ubiquitylation and proteasomal degradation of EID1. Genes Cells 20, 667-674.
Yamazoe, S., Tom, J., Fu, Y., Wu, W., Zeng, L., Sun, C., Liu, Q., Lin, J., Lin, K., Fairbrother, W. J., et al. (2020). Heterobifunctional Molecules Induce Dephosphorylation of Kinases-A Proof of Concept Study. J Med Chem 63, 2807-2813.
Yip, M. C. J., Bodnar, N. O., and Rapoport, T. A. (2020). Ddi1 is a ubiquitin-dependent protease. PNAS 117, 7776-7781.
Zhang, C., Li, X., Adelmant, G., Dobbins, J., Geisen, C., Oser, M. G., Wucherpfenning, K. W., Marto, J. A., and Kaelin, W. G. (2015). Peptidic degron in EID1 is recognized by an SCF E3 ligase complex containing the orphan F-box protein FBXO21. PNAS 112, 15372-15377.

1. A method of identifying a proximity effector polypeptide, the method comprising:
transducing an ORFeome library into a plurality of cells, the ORFeome library encoding a plurality of ORFs, wherein each of the ORFs is fused to a targeting moiety that binds or can be induced to bind to the target polypeptide directly or indirectly;
expressing the plurality of ORFs of the ORFeome library in the transduced plurality of cells, under conditions for the targeting moiety to interact with the target polypeptide; and
determining whether any of the plurality of ORFs is a proximity effector polypeptide by measuring abundance, optionally total abundance, cell surface abundance or subcellular abundance, of the target polypeptide in cells expressing any of the plurality of ORFs compared to a control and/or detecting whether any of the plurality of ORFs is depleted or enhanced in the transduced plurality of cells compared to a control;
wherein the transduced plurality of cells recombinantly expresses the target polypeptide and optionally expresses a first fluorescent polypeptide, wherein the target polypeptide is a second fluorescent polypeptide, endogenous protein, or a fusion polypeptide fused to a second fluorescent polypeptide epitope tag, an antibiotic resistance protein and/or a negative selection marker; and
wherein an ORF encodes a proximity effector polypeptide when the ORF increases or decreases the target polypeptide abundance compared to control or is depleted or enhanced in the transduced plurality of cells compared to the control.

2. The method of claim 1, wherein measuring abundance of the target polypeptide comprises i) determining whether the target polypeptide expressed in the transduced plurality of cells has decreased or has increased relative to a control, or ii) determining whether any of the plurality ORFs increased cell surface levels of the target polypeptide compared to a control.

3. The method of claim 1 or 2, wherein the proximity effector polypeptide is a degrader of the target polypeptide when the target polypeptide is decreased compared to control or the proximity effector polypeptide is a stabilizer of the target polypeptide when the target polypeptide has increased compared to control;
wherein the proximity effector polypeptide is a lethal polypeptide when depleted or decreased in the transduced plurality of cells or is a growth inducing polypeptide when enhanced in the transduced plurality of cells compared to control; or
wherein the proximity effector polypeptide is a protein trafficking polypeptide when the proximity effector increases cell surface levels of the target polypeptide compared to control.

4. The method of any one of claims 1 to 3, wherein when the target polypeptide is or is fused to a second fluorescent polypeptide, the determining comprises isolating a fraction of the transduced plurality of cells with a selected second fluorescent polypeptide: first fluorescent polypeptide ratio and ORF sequencing one or more of the plurality of ORFs in the fraction;
wherein when the target polypeptide is fused to an epitope tag, the determining comprises measuring abundance of the target polypeptide with an epitope tag binding protein, optionally an antibody;
wherein when the target polypeptide is an endogenous target, the determining comprises measuring abundance of the target polypeptide with a target polypeptide binding protein, optionally an antibody;
wherein when the target polypeptide is fused to the antibiotic selection protein, the determining comprises isolating a fraction of the transduced plurality of cells that survive antibiotic treatment and ORF sequencing one or more of the ORFs in the fraction; or
wherein when the target polypeptide is fused to the negative selection marker, optionally thymidine kinase, the determining comprises isolating a fraction of the transduced plurality of cells that survive negative selection treatment and ORF sequencing one or more of the ORFs in the fraction.

5. The method of any one of claims 1 to 4, wherein the plurality of cells is a cell line and the method further comprises generating the cell line by introducing a nucleic acid encoding the target polypeptide, and optionally the first fluorescent polypeptide, optionally wherein the target polypeptide and first fluorescent polypeptide are in a construct comprising an IRES or cleavage site therebetween.

6. The method of any one of claims 1 to 5, wherein the ratio of the second fluorescent polypeptide to the first fluorescent polypeptide is determined using a method comprising flow cytometry.

7. The method of any one of claims 1 to 6, wherein the first or second fluorescent polypeptide(s) is/are RFP, YFP, mCherry, mCitrine, mNeonGreen, mScarlet, BFP and/or GFP.

8. The method of any one of claims 1 to 7, wherein the first fluorescent polypeptide is GFP and the second fluorescent polypeptide is BFP or wherein the first fluorescent polypeptide is BFP and the second fluorescent polypeptide is GFP.

9. The method of any one of claims 1 to 5, wherein the target polypeptide is fused to the antibiotic resistance protein or the negative selection marker.

10. The method of any one of claims 1 to 4 or 9, wherein the ORF identified in a cell that survives antibiotic treatment is a effector.

11. The method of any one of claims 1 to 10, wherein the determining comprises measuring growth of the transduced plurality of cells and ORFs identified in a cell of the transduced plurality of cells that enhances or decreases cell proliferation compared to a control is a proximity effector polypeptide.

12. The method of any one of claims 1 to 4, or 9 to 11, wherein the antibiotic resistance protein is puromycin acetyltransferase.

13. The method of claim 12, wherein the method comprises treating the transduced plurality of cells with puromycin during the step of expressing the plurality of ORFs of the ORFeome library, under conditions for the targeting moiety to interact with the target polypeptide.

14. The method of any one of claims 1 to 4, 9 or 11, wherein the negative selection marker is thymidine kinase, mutant deoxycytodine kinase or thymidylate kinase.

15. The method of claim 14, wherein when the negative selection marker is thymidine kinase the method comprises treating the transduced plurality of cells with ganciclovir during the step of expressing the plurality of ORFs of the ORFeome library, under conditions for the targeting moiety to interact with the target polypeptide, wherein survival of a cell when exposed to ganciclovir indicates that the level of the target polypeptide is decreased.

16. The method of any one of claims 1 to 15, wherein the plurality of cells are transduced to maintain on average about >300, >400 or on average about >500 fold coverage of the ORFeome library.

17. The method of any one of claims 1 to 16, wherein the method further comprises testing identified proximity effector polypeptides in an individual proximity effector assay, optionally when the proximity effector polypeptide is identified as a stabilizer or degrader, expressing the putative proximity effector polypeptide identified as the stabilizer or degrader in a test cell expressing the target polypeptide and determining whether the level of the target polypeptide in the test cell has decreased or has increased.

18. The method of any one of claims 1 to 12, the at the proximity effector polypeptide is a plurality of proximity effector polypeptides.

19. The method of any one of claims 1 to 5, for identifying a proximity effector polypeptide that is a lethal polypeptide or a growth inducing polypeptide, wherein the determining comprises determining whether the ORF has caused death or induced proliferation of at least one cell of the transduced plurality of cells and/or is depleted or enhanced in the transduced plurality of cells compared to a control, and wherein the proximity effector polypeptide is a lethal polypeptide when the proximity effector polypeptide causes death in at least one cell of the transduced plurality of cells and/or wherein it is depleted in the transduced plurality of cells and the putative proximity effector polypeptide is a growth inducing polypeptide when the proximity effector polypeptide induces proliferation in at least one cell and/or is enhanced in the transduced plurality of cells compared to control.

20. The method of claim 19, wherein the determining I comprises identifying the ORFs depleted in the transduced plurality of cells, optionally comprising sequencing the plurality of ORFs in the transduced plurality of cells that survived and comparing a reference of the plurality of ORFs in the ORFeome library to determine ORFs are or are not present.

21. The method of claim 19 or 20, wherein the target polypeptide is an oncogenic polypeptide, a regulator of apoptosis, a regulator of autophagy, or a regulator of mitophagy.

22. The method of claim 21, wherein the target polypeptide is a RAS polypeptide, MYC, or EWSR-FLI1.

23. The method of claim 22, wherein the target polypeptide is the RAS polypeptide, optionally KRAS.

24. The method of any one of claims 1 to 5 for identifying a proximity effector polypeptide that is a protein trafficking polypeptide, wherein the determining comprises measuring cell surface levels of the target polypeptide, wherein the proximity effector polypeptide is a protein trafficking polypeptide when it increases the cell surface levels of the target polypeptide.

25. The method of claim 24, wherein the target polypeptide is a MHC class I polypeptide.

26. The method of claim 24, wherein the target polypeptide is a mutant cell surface polypeptide, optionally provided Table 2.

27. The method of claim 26, wherein the mutant cell surface polypeptide is CFTR delta508.

28. The method of any one of claims 1 to 27, wherein the target polypeptide comprises EGFP-AB1, Rluc, FUS S525L, NRAS, DNAJA3, BRAF, LAMP1, TDP43 Q311K, CD63, H2B, EGFR, DNAJB11, WDR5, RAS, MYC, or EWSR-FLI1, EWSR1, SMARCA2/4, or PARP1, PD1/PD-L1, JAK, FUS, TDP43, a-synuclein, amyloid beta precursor protein, HTT, prion protein, p53, PTEN, a CFTR variant, and/or dystrophin variant.

29. The method of any one of claims 1 to 28, wherein the targeting moiety is a nanobody, ligand, interaction peptide or an antibody that binds the target polypeptide.

30. The method of any one of claims 1 to 29, wherein targeting moiety is the nanobody.

31. The method of any one of claims 1 to 29, wherein the targeting moiety is an interaction peptide selected from ABI1, FKBP, FRB, mutant FRB, GID1, GAI, and/or PYR1.

32. The method any one of claims 1 to 29, wherein the target polypeptide is a fusion polypeptide comprising the interaction peptide that interacts with the targeting moiety.

33. The method of claim 32, wherein the fusion polypeptide comprises ABI1, FKBP, FRB, mutant, FRB, GID1, GAI, PYL1, ALFA tag and/or PYR1.

34. The method of any one of claims 31 to 33, wherein the method comprises use of a chemical inducer.

35. The method of claim 34, wherein when the target polypeptide comprises ABI1, the targeting moiety comprises PYR1 or PYL1, and the chemical inducer is mandipropamid or abscisic acid.

36. The method of claim 34, wherein when the target polypeptide comprises PYR1, the targeting moiety comprises ABI1, and the chemical inducer is mandipropamid or abscisic acid.

37. The method of claim 34, wherein when the target polypeptide comprises ABI1, the targeting moiety comprises PYL1, and the chemical inducer is abscisic acid.

38. The method of claim 34, wherein when the target polypeptide comprises FKBP, the targeting moiety comprises FRB, and the chemical inducer is rapamycin.

39. The method of claim 34, wherein when the target polypeptide comprises FRB, the targeting moiety comprises FKBP, and the chemical inducer is rapamycin.

40. The method of claim 34, wherein when the target polypeptide comprises FKBP, the targeting moiety comprises mutant FRB, and the chemical inducer is a rapalog, optionally AP21967.

41. The method of claim 34, wherein when the target polypeptide comprises mutant FRB, the targeting moiety comprises FKBP, and the chemical inducer is a rapalog, optionally AP21967.

42. The method of claim 34, wherein when the target polypeptide comprises GID1, the targeting moiety comprises GAI, and the chemical inducer is gibberellic acid.

43. The method of claim 34, wherein when the target polypeptide comprises mutant GAI, the targeting moiety comprises GID1, and the chemical inducer is gibberellic acid.

44. The method of any one of claims 1 to 43, wherein the method further comprises performing a screening assay for identifying a ligand, optionally a small molecule binder, for at least one recombinant proximity effector polypeptide identified.

45. A screening assay for identifying a ligand, optionally a small molecule binder, of at least one recombinant proximity effector polypeptide, the screening assay comprising:
contacting the at least one recombinant proximity effector polypeptide with a small molecule library optionally in a high-throughput screening assay, wherein the proximity effector polypeptide is selected from Table 4, 5, 6 or 7,
assessing whether binding has occurred between the recombinant proximity effector polypeptide and one or more small molecule(s) of the small molecule library,
wherein the one or more molecule(s) which have bound to the at least one recombinant proximity effector polypeptide is a ligand, optionally a small molecule binder, of the at least one recombinant proximity effector polypeptide;
preferably wherein the proximity effector polypeptide is selected from GMCL1, FBXL15, PJA1, RNF115, DZIP3, RNF125, FBXO3, RNF185, RNF8, RNF183, RCHY1, KBTBD7, TRIM31, CISH, SOCS5, TRIM39, RNF144B, FBXO40, KLHL6, FBXO11, GAN, FBXL14, FBXW5, RNF111, FBXL12, BTRC, or RNF126 or selected from FBXL12, FBXL14, FBXL15, KLHDC2, KLHL6, KBTBD7, ZER1, UBE2B or KLHL40.

46. The screening assay of claim 45, wherein the assay further comprises contacting a target polypeptide with the small molecule library and determining whether binding has occurred between the target polypeptide and one or more small molecule(s) of the small molecule library.

47. The screening assay of claim 45 or 46, wherein the assessing step is performed using surface plasmon resonance (SPR), nuclear magnetic resonance (NMR) spectroscopy, differential scanning fluorimetry (DSF), thermal shift assay (TSA), isothermal titration calorimetry (ITC), microscale thermophoresis (MST), biolayer interferometry (BLI), X-ray crystallography, DNA-Encoded Library (DEL) screens, affinity selection-mass spectrometry (AS-MS), or covalent fragment screens.

48. The screening assay of claim 45, wherein the assay further comprises identifying whether the small molecule binder of the recombinant proximity effector polypeptide is a molecular glue by determining whether the recombinant proximity effector and the target polypeptide interact in the presence of the small molecule binder.

49. The screening assay of claim 48, wherein the determining step is performed using a luciferase complementation, a yeast two-hybrid assay, an AlphaScreen, a yeast mating based interaction assay, fluorescence resonance energy transfer microscopy (FRET), or time-resolved FRET (TR-FRET), wherein the small molecule binder is a molecular glue if it interacts or is capable of interacting with the recombinant proximity effector polypeptide and the target polypeptide simultaneously.

50. The screening assay of any one of claims 45 to 49, wherein the at least one recombinant proximity effector polypeptide has been identified using the methods of any one of claims 1 to 44 and/or wherein the method comprises making a product with the ligand, optionally small molecule binder, optionally a therapeutic product.

51. A method of making a heterobifunctional molecule, the method comprising:
identifying a ligand, optionally a small molecule binder, of an effector polypeptide and a small molecule binder of a target polypeptide using the methods of any one of claims 45 to 47, and
coupling the ligand optionally the small molecule binder of the effector polypeptide and the small molecule binder of the target polypeptide optionally via a linker.

52. The method of claim 51, the method further comprising assessing whether the effector polypeptide and the target polypeptide interact in the presence of the heterobifunctional molecule.

53. The method of claim 52, wherein the assessing step is performed using a luciferase complementation, a yeast two-hybrid assay, an AlphaScreen, a yeast mating based interaction assay, fluorescence resonance energy transfer microscopy (FRET), or time-resolved FRET (TR-FRET).

54. A process for modulating a target polypeptide in at least one cell, the method comprising: expressing the proximity effector polypeptide provided in Table 4, 5, 6 or 7 in the at least one cell, the at least one proximity effector polypeptide fused to a targeting moiety, preferably wherein the proximity effector polypeptide is selected from GMCL1, FBXL15, PJA1, RNF115, DZIP3, RNF125, FBXO3, RNF185, RNF8, RNF183, RCHY1, KBTBD7, TRIM31, CISH, SOCS5, TRIM39, RNF144B, FBXO40, KLHL6, FBXO11, GAN, FBXL14, FBXW5, RNF111, FBXL12, BTRC, or RNF126 or selected from FBXL12, FBXL14, FBXL15, KLHDC2, KLHL6, KBTBD7, ZER1, UBE2B or KLHL40.

55. The process of claim 54 wherein the proximity effector polypeptide is at least one degrader.

56. The process of claim 54 wherein the proximity effector polypeptide is at least one stabilizer.

57. The process of claim 4455, wherein the at least one degrader polypeptide is selected from UBE2B, UBE2A, FBXL12, FBXL14, FBXL15, GABARAP, GABARAPL2, MAP1LC3A, KLHL6, KBTBD7, ZER1 and/or KLHDC2.

58. The process of claim 55, wherein the at least one degrader is selected from GMCL1, FBXL15, PJA1, RNF115, DZIP3, RNF125, FBXO3, RNF185, RNF8, RNF183, RCHY1, KBTBD7, TRIM31, CISH, SOCS5, TRIM39, RNF144B, FBXO40, KLHL6, FBXO11, GAN, FBXL14, FBXW5, RNF111, FBXL12, BTRC, ZER1 and/or RNF126.

59. The process of claim 55, wherein the at least one degrader polypeptide is selected from FBXL12, FBXL14, FBXL15, KLHDC2, KLHL6, KBTBD7, ZER1 and/or UBE2B.

60. The process of claim 456, wherein the at least one stabilizer polypeptide is selected from KLHL40, KLHL41, DDI1, and/or PRPS2.

61. The process of claim 56 or 60, wherein the at least one stabilizer polypeptide is KLHL40.

62. The process of any one of claims 54 to 61, wherein the target polypeptide is an oncogene polypeptide, oncogenic fusion polypeptide, synthetic lethal target, immunology/immune-oncology target, dominant gain-of-function disease variant, tumor suppressor, or unstable disease variant.

63. The process of claim 62 wherein the oncogene polypeptide or oncogenic fusion polypeptide is or comprises RAS, MYC, or EWSR-FLI1.

64. The process of claim 62, wherein the synthetic lethal target is EWSR1, SMARCA2/4, or PARP1.

65. The process of claim 62, wherein the immunology/immune-oncology target is PD1/PD-L1 or JAK.

66. The process of claim 62, wherein the dominant gain-of-function disease variant is FUS, TDP43, a-synuclein, amyloid beta precursor protein, HTT, or prion protein.

67. The process of claim 62, wherein the tumor suppressor is p53 or PTEN.

68. The process of claim 62, wherein the unstable disease variant is CFTR mutations or dystrophin variants.

69. The process of claim 62 wherein the proximity effector is KLHL40 or KLHL41 and the target polypeptide is a loss of stability variant in muscular dystrophy.

70. The process of claim 62 wherein the target polypeptide is BCR-ABL.

71. The process of any one of claims 54 to 70, wherein the targeting moiety is a nanobody, ligand, interaction peptide or an antibody.

72. A fusion polypeptide comprising a proximity effector polypeptide selected from Table 4, 5, 6 or 7 and a targeting moiety that binds a target polypeptide, preferably wherein the proximity effector polypeptide is selected from GMCL1, FBXL15, PJA1, RNF115, DZIP3, RNF125, FBXO3, RNF185, RNF8, RNF183, RCHY1, KBTBD7, TRIM31, CISH, SOCS5, TRIM39, RNF144B, FBXO40, KLHL6, FBXO11, GAN, FBXL14, FBXW5, RNF111, FBXL12, BTRC, or RNF126 or selected from FBXL12, FBXL14, FBXL15, KLHDC2, KLHL6, KBTBD7, ZER1, UBE2B or KLHL40.

73. The fusion polypeptide of claim 72, wherein the effector polypeptide is selected from ZER1 FBXL12, FBXL14, FBXL15, KLHDC2, KLHL6, KBTBD7, UBE2B or KLHL40.

74. The fusion polypeptide of claim 72, wherein the proximity effector polypeptide is selected from UBE2B, UBE2A, ZER1, FBXL12, FBXL14, FBXL15, GABARAP, GABARAPL2, MAP1LC3A, KLHL6, KBTBD7, KLHDC2, KLHL40, KLHL40:BTB domain of KLHL6 fusion, or PRNP: residues 194-223 of FCGR3B fusion.

75. The fusion polypeptide of claim 72, wherein the effector polypeptide is UBE2B, ZER1 KLHL40, KLHL41, DDI1, or PRPS2.

76. The fusion polypeptide of claim 72, wherein the fusion polypeptide comprises a proximity effector polypeptide that is a degrader.

77. The fusion polypeptide of claim 76, wherein the proximity effector polypeptide is selected from GMCL1, FBXL15, PJA1, RNF115, DZIP3, RNF125, FBXO3, RNF185, RNF8, RNF183, RCHY1, KBTBD7, TRIM31, CISH, SOCS5, TRIM39, RNF144B, FBXO40, KLHL6, FBXO11, GAN, FBXL14, FBXW5, RNF111, FBXL12, BTRC, ZER1 or RNF126.

78. The fusion polypeptide of claim 72, wherein the proximity effector polypeptide is selected from FBXL12, FBXL14, FBXL15, KLHDC2, KLHL6, KBTBD7, ZER1 or, UBE2B.

79. The fusion polypeptide of claim 72, wherein the proximity effector polypeptide is a stabilizer.

80. The fusion polypeptide of claim 79 wherein the stabilizer is KLHL40.

81. The fusion polypeptide of any one of claims 72 to 80, wherein the targeting moiety is a nanobody, ligand, interaction peptide or an antibody that binds the target polypeptide.

82. The fusion polypeptide of any one of claims 72 to 81, wherein the targeting moiety is a nanobody, optionally vhhGFP or ALFA-tag nanobody.

83. The fusion polypeptide of any one of claims 72 to 82, wherein the target polypeptide is selected from an oncogene polypeptide, oncogenic fusion polypeptide, synthetic lethal target, immunology/immune-oncology target, dominant gain-of-function disease variant, tumor suppressors, or unstable disease variant.

84. The fusion polypeptide of claim 83 wherein the oncogene polypeptide or oncogenic fusion polypeptide is RAS, MYC, or EWSR-FLI1.

85. The fusion polypeptide of claim 83, wherein the synthetic lethal target is EWSR1, SMARCA2/4, or PARP1.

86. The fusion polypeptide of claim 63, wherein the immunology/immune-oncology target is PD1/PD-L1 or JAK.

87. The fusion polypeptide of claim 83, wherein the dominant gain-of-function disease variant is FUS, TDP43, a-synuclein, amyloid beta precursor protein, HTT, or prion protein.

88. The fusion polypeptide of claim 83, wherein the tumor suppressor is p53 or PTEN.

89. The fusion polypeptide of claim 83, wherein the unstable disease variant is a CFTR variant or dystrophin variant.

90. The fusion polypeptide of any one of claims 72 to 82, wherein the target polypeptide is selected from EGFP-AB1, ABI1, Rluc, FUS S525L, NRAS, DNAJA3, BRAF, LAMP1, TDP43, Q311K, CD63, H2B, EGFR, DNAJB11 or WDR5.

91. The fusion polypeptide of any one of claims 72 to 90 for use in making a medicament.

92. The fusion polypeptide for use of claim 91, wherein the proximity effector is KLHL40 or KLHL41 and the target polypeptide is a loss of stability variant and the medicament is for treating muscular dystrophy.

93. The fusion polypeptide for use of claim 91, wherein the target polypeptide is BCR-Abl.

94. A nucleic acid encoding the fusion polypeptide of any one of claims 72 to 93.

95. A vector comprising the nucleic acid of claim 94.

96. A method of identifying a proximity effector polypeptide, the method comprising:
transducing an ORFeome library into a plurality of cells, the ORFeome library encoding a plurality of ORFs, wherein each of the ORFs is fused to a nanobody, and optionally an interaction peptide, that binds to a target polypeptide;
expressing the plurality of ORFs of the ORFeome library in the transduced plurality of cells, under conditions for the nanobody, and optionally the interaction peptide, to interact with the target polypeptide; and
determining whether any of the plurality of ORFs is a proximity effector polypeptide by measuring abundance of the target polypeptide in cells expressing any of the plurality of ORFs compared to control;
wherein the transduced plurality of cells recombinantly expresses a fluorescent polypeptide, optionally blue fluorescent protein (BFP) and expresses the target polypeptide, wherein the target polypeptide is another fluorescent polypeptide, optionally green fluorescent protein (GFP) or an endogenous protein fused to another fluorescent polypeptide, optionally GFP, optionally fused to a complementary interaction polypeptide; and wherein an ORF encodes a proximity effector polypeptide when the ORF increases or decreases the target polypeptide abundance compared to control.

97. The method of claim 96, wherein the ORF is fused to a nanobody and an interaction peptide that binds to a target polypeptide.

98. The method of claim 96 or 97, wherein the target polypeptide is a fluorescent polypeptide, optionally GFP.

99. A method of identifying a proximity effector polypeptide, the method comprising:
transducing an ORFeome library into a plurality of cells, the ORFeome library encoding a plurality of ORFs, wherein each of the ORFs is fused to an interaction peptide, that binds to a target polypeptide;
expressing the plurality of ORFs of the ORFeome library in the transduced plurality of cells, under conditions for the interaction peptide to interact with the target polypeptide; and
determining whether any of the plurality of ORFs is a proximity effector polypeptide by measuring abundance of the target polypeptide in cells expressing any of the plurality of ORFs compared to control;
wherein the transduced plurality of cells recombinantly expresses a fluorescent polypeptide, optionally blue fluorescent protein (BFP), and expresses the target polypeptide, wherein the target polypeptide is another fluorescent polypeptide, optionally green fluorescent protein (GFP) or an endogenous protein fused to another fluorescent protein, optionally GFP, fused to a complementary interaction peptide; and wherein an ORF encodes a proximity effector polypeptide when the ORF increases or decreases the target polypeptide abundance compared to control.

100. The method of any one of claims 96 to 99, wherein the conditions for the interaction peptide to interact with the target polypeptide is in the presence of a chemical inducer.

101. The method of any one of claims 96 to 99, wherein the interaction peptide is PYL1 and the complementary interaction peptide is ABI or the interaction peptide is ABI and the complementary interaction peptide is PYL1.

102. The method of claim 101, wherein the chemical inducer is abscisic acid.

103. The method of any one of claims 96 to 98, the method further comprising performing the method of any one of claims 99 to 102 and comparing the comparing results from the method of any one of claims 96 to 102 and results of the method of any one of claims 99 to 102 and identifying ORFs that were identified as being proximity effector polypeptides in both methods.


SEQUENCE LISTING (41 sequences)

SEQ ID NO: 1 - DNA, 24 bp, synthetic construct
  source 1..24: mol_type = other DNA
cgactcactatagggagacccaag

SEQ ID NO: 2 - DNA, 22 bp, synthetic construct
  source 1..22: mol_type = other DNA
attcatcttgcgttggtgctcc

SEQ ID NO: 3 - DNA, 24 bp, synthetic construct
  source 1..24: mol_type = other DNA
gccaccagactccaccagttggac

SEQ ID NO: 4 - DNA, 21 bp, synthetic construct
  source 1..21: mol_type = other DNA
cagtgtggtggaattctgcag

SEQ ID NO: 5 - DNA, 20 bp, synthetic construct
  source 1..20: mol_type = other DNA
ccgccactgtgctggatatc

SEQ ID NO: 6 - protein, 33 aa, Homo sapiens
SFSPPGYQVSFCLVMVLLFAVDTGLYFSVKTNI

SEQ ID NO: 7 - protein, 28 aa, Homo sapiens
GYQVSFCLVMVLLFAVDTGLYFSVKTNI

SEQ ID NO: 8 - protein, 23 aa, Homo sapiens
FCLVMVLLFAVDTGLYFSVKTNI

SEQ ID NO: 9 - protein, 18 aa, Homo sapiens
VLLFAVDTGLYFSVKTNI

SEQ ID NO: 10 - protein, 13 aa, Homo sapiens
VDTGLYFSVKTNI

SEQ ID NO: 11 - protein, 8 aa, Homo sapiens
YFSVKTNI

SEQ ID NO: 12 - protein, 28 aa, Homo sapiens
SFSPPGYQVSFCLVMVLLFAVDTGLYFS

SEQ ID NO: 13 - protein, 23 aa, Homo sapiens
SFSPPGYQVSFCLVMVLLFAVDT

SEQ ID NO: 14 - protein, 18 aa, Homo sapiens
SFSPPGYQVSFCLVMVLL

SEQ ID NO: 15 - protein, 13 aa, Homo sapiens
SFSPPGYQVSFCL

SEQ ID NO: 16 - protein, 8 aa, Homo sapiens
SFSPPGYQ

SEQ ID NO: 17 - protein, 6 aa, Homo sapiens
SFSPPG

SEQ ID NO: 18 - protein, 33 aa, Homo sapiens
AAAAAGYQVSFCLVMVLLFAVDTGLYFSVKTNI

SEQ ID NO: 19 - protein, 33 aa, Homo sapiens
SFSPPAAAAAFCLVMVLLFAVDTGLYFSVKTNI

SEQ ID NO: 20 - protein, 33 aa, Homo sapiens
SFSPPGYQVSAAAAAVLLFAVDTGLYFSVKTNI

SEQ ID NO: 21 - protein, 33 aa, Homo sapiens
SFSPPGYQVSFCLVMAAAAAVDTGLYFSVKTNI

SEQ ID NO: 22 - protein, 33 aa, Homo sapiens
SFSPPGYQVSFCLVMVLLFAAAAAAYFSVKTNI

SEQ ID NO: 23 - protein, 33 aa, Homo sapiens
SFSPPGYQVSFCLVMVLLFAVDTGLAAAAATNI

SEQ ID NO: 24 - protein, 33 aa, Homo sapiens
SFSPPGYQVSFCLVMVLLFAVDTGLYFSVKAAA

SEQ ID NO: 25 - protein, 49 aa, Homo sapiens
XXLAAXSXYFXAMFXXXXXEXXXXXXXXXGVXXXXXXXXIXFXYTXXXX

SEQ ID NO: 26 - protein, 49 aa, Homo sapiens
VVLASSSPVFKAMFTNGLREQGMEVVSIEGIHPKVMERLIEFAYTASIS

SEQ ID NO: 27 - protein, 49 aa, Homo sapiens
VVLAACSPYFCAMFYGDMSESKAKKIEIKDVDGQTLSKLIDYIYTAEIE

SEQ ID NO: 28 - protein, 49 aa, Homo sapiens
VVLAAASNYFRAMFCNDLKEKYEKRIIIKGVDAETMHTLLDYTYTSKAL

SEQ ID NO: 29 - protein, 49 aa, Homo sapiens
AMMASASDYFKAMFTGGMKEQDLMCIKLHGVNKVGLKKIIDFIYTAKLS

SEQ ID NO: 30 - protein, 55 aa, Homo sapiens
SVLAAATEYFTPLLSGQFSESRSGRVEMRKWSSEPGPEPDTVEAVIEYMYTGRIR

SEQ ID NO: 31 - protein, 49 aa, Homo sapiens
IVLAACSDYFCAMFTSELSEKGKPYVDIQGLTASTMEILLDFVYTETVH

SEQ ID NO: 32 - protein, 49 aa, Homo sapiens
VMMASASDYFKAMFTGGMKEQDLMCIKLHGVSKVGLRKIIDFIYTAKLS

SEQ ID NO: 33 - protein, 49 aa, Homo sapiens
IVLAASIPYFHAMFTNDMMECKQDEIVMQGMDPSALEALINFAYNGNLA

SEQ ID NO: 34 - protein, 49 aa, Homo sapiens
VILSACSPYFRAMFTGELAESRQTEVVIRDIDERAMELLIDFAYTSQIT

SEQ ID NO: 35 - protein, 49 aa, Homo sapiens
ILLAASCDYFRGMFAGGLKEMEQEEVLIHGVSYNAMCQILHFIYTSELE

SEQ ID NO: 36 - protein, 47 aa, Homo sapiens
LVLAACSPYFRARFLAEPERAGELHLEEVSPDVVAQVLHYLYTSEIA

SEQ ID NO: 37 - protein, 49 aa, Homo sapiens
LILSACSPYFREYFLSEIDEAKKKEVVLDNVDPAILDLIIKYLYSASID

SEQ ID NO: 38 - protein, 56 aa, Homo sapiens
RKLIEQSDYFRALYRSGMREALSQEAGGPEVQQLRGLSAPGLRLVLDFINAGGARE

SEQ ID NO: 39 - protein, 49 aa, Homo sapiens
AILAARSPVFSAMFEHEMEESKKNRVEINDVEPEVFKEMMCFIYTGKAP

SEQ ID NO: 40 - protein, 30 aa, Homo sapiens
AAPTRTALAHSALSLGLALSLLAVILAPSL

SEQ ID NO: 41 - protein, 20 aa, Homo sapiens
VAPTAGLAALLLAVAAGVLL